(19) United States
(12) Patent Application Publication
(10) Pub. No.: US 2026/0053801 A1
(43) Pub. Date: Feb. 26, 2026

(54) METHOD FOR TREATING AUTOPHAGY DEPENDENT CANCER WITH A PIKFYVE INHIBITOR

(71) Applicant: The Regents of the University of Michigan, Ann Arbor, MI (US)

(72) Inventors: Arul M. Chinnaiyan, Ann Arbor, MI (US); Yuanyuan Qiao, Ann Arbor, MI (US)

(21) Appl. No.: 19/307,723

(22) Filed: Aug. 22, 2025

Related U.S. Application Data
(63) Continuation of application No. PCT/US2024/017088, filed on Feb. 23, 2024.
(60) Provisional application No. 63/447,763, filed on Feb. 23, 2023.

(51) Int. Cl. A61K 31/506; A61K 31/519; A61P 35/00

(52) U.S. Cl. CPC A61K 31/506; A61K 31/519; A61P 35/00

(57) ABSTRACT

Provided herein are methods for treating autophagy dependent cancers. In particular, provided herein are methods for treating neuroendocrine prostate cancer (NEPC), pancreatic ductal adenocarcinoma (PDAC), pancreatic neuroendocrine tumors (PNETs), pancreatic neuroendocrine carcinomas (NECs), colorectal cancer (CRC), and non-small cell lung cancer (NSCLC) comprising administering a therapeutic agent, e.g., ESK981, that inhibits PIKfyve and, optionally, a RAS inhibitor.

### CROSS REFERENCE TO RELATED APPLICATIONS

The present application is a continuation of International Patent Application No. PCT/US2024/017088, filed Feb. 23, 2024, which claims priority to U.S. Provisional Application No. 63/447,763, filed Feb. 23, 2023, which are incorporated herein by reference in their entireties.

### STATEMENT OF GOVERNMENTAL INTEREST

This invention was made with government support under CA186786, and CA231996 awarded by the National Institutes of Health. The government has certain rights in the invention.

### BACKGROUND

### Field of Invention

This disclosure provides methods for treating autophagy dependent cancers. In particular, provided herein are methods for treating neuroendocrine prostate cancer (NEPC), pancreatic ductal adenocarcinoma (PDAC), pancreatic neuroendocrine tumors (PNETs), pancreatic neuroendocrine carcinomas (NECs), colorectal cancer (CRC), or non-small cell lung cancer (NSCLC) comprising administering a therapeutic agent, e.g., ESK981 or apilimod, that inhibits PIKfyve and, optionally, a RAS inhibitor.

### Background

Autophagy is an evolutionarily conserved, ordered pathway of degradation of intracellular materials required to sustain cellular homeostasis. Klionsky et al., Embo j. 2021; 40(19):e108863. Autophagy is often exacerbated under stress conditions like nutrient deprivation, protein aggregation, organelle senescence, and hypoxia that commonly occur in different types of cancers, thereby creating a potential therapeutic vulnerability to autophagy inhibitors in certain contexts. Poillet-Perez et al., Nature. 2018; 563(7732):569-73; Nguyen et al., Oncogene. 2014; 33(36):4521-30; Yang et al., Genes Dev. 2011; 25(7):717-29; Russell et al., Embo j. 2022; 41(13):e110031; Levy et al., Nat Rev Cancer. 2017; 17(9):528-42.

A simplified overview of the autophagy pathway involves formation of double-membraned autophagosomes that encapsulate cytoplasmic material targeted for degradation, followed by fusion of autophagosomes with lysosomes, which contain enzymes that degrade the encapsulated contents so that nutrients and metabolites can be recycled back into the cytoplasm. Russell et al., Embo j. 2022; 41(13):e110031. This process requires an enhanced cellular demand for lysosome function and biogenesis to match the increase in autophagosome formation. TRPML1, a key player in lysosomal trafficking, is a cation channel on the lysosomal membrane that releases Ca2⁺ from the lumen into the cytosol in response to trafficking cues, such as changes in levels of the phosphoinositide PI(3,5)P2, Dong et al., Nature Communications. 2010; 1, to increase lysosome proteolytic activity and clearance of lysosomal storage. Wang et al., PNAS. 2015; 112(11):E1373-E81. PIKfyve (FYVE finger-containing phosphatidylinositol-3-phosphate 5-kinase) is a lipid kinase located on endosomal membranes and is the sole enzyme responsible for phosphorylating PI3P to generate PI(3,5)P2. Lees et al., Molecular Cell. 2020; 80(4):736; Zolov et al., PNAS. 2012; 109(43):17472-7. Direct inhibition of PIKfyve results in lysosome enlargement and inhibition of autophagic flux, Choy et al., J Cell Sci. 2018; 131(10).; Gayle et al., Blood. 2017; 129(13):1768-78; Sharma et al., Autophagy. 2019; 15(10):1694-718, and PIKfyve inhibitors have, thus, shown promise as anti-cancer agents in non-Hodgkin's lymphoma, Gayle et al., Blood. 2017; 129(13):1768-78, and multiple myeloma. De Campos et al., Haematologica. 2020; 105(6):1641-9; De Campos et al., Blood. 2017; 130. There exists a need in the art to identify other cancers that may be responsive to PIKfyve inhibition.

### BRIEF SUMMARY

In one aspect, the present disclosure provides methods of treating prostate cancer, pancreatic cancer, colorectal cancer (CRC), or non-small cell lung cancer (NSCLC) in a subject in need thereof, the method comprising administering to the subject a therapeutically effective amount of ESK981, or a pharmaceutically acceptable salt and/or solvate thereof. In another aspect, the present disclosure provides methods of treating prostate cancer, pancreatic cancer, colorectal cancer, or non-small cell lung cancer in a subject in need thereof, the method comprising administering to the subject a therapeutically effective amount of ESK981, or a pharmaceutically acceptable salt and/or solvate thereof, in combination with a therapeutically effective amount of a KRAS-MAPK inhibitor.

In another aspect, the present disclosure provides methods of treating prostate cancer, pancreatic cancer, colorectal cancer, or non-small cell lung cancer in a subject in need thereof, the method comprising administering to the subject a therapeutically effective amount of ESK981, or a pharmaceutically acceptable salt and/or solvate thereof, in combination with a therapeutically effective amount of a RAS inhibitor.

In another aspect, the prostate cancer is NEPC.

In another aspect, the pancreatic cancer is PDAC, PNETs, or NECs.

In another aspect, the prostate cancer, pancreatic cancer, colorectal cancer, or non-small cell lung cancer is characterized as having a KRAS mutation and/or a Trp53 mutation.

In another aspect, the present disclosure provides a method of treating a subject having NEPC, PDAC, PNETs, NECs, CRC, or NSCLC, the method comprising:
(a) determining whether a KRAS mutation and/or a Trp53 mutation is present or absent in a biological sample taken from the subject; and
(b) administering a therapeutically effective amount of ESK981, or a pharmaceutically acceptable salt and/or solvate thereof, to the subject if a KRAS mutation and/or a Trp53 mutation is present in the biological sample.

In another aspect, the present disclosure provides a method, comprising administering a therapeutically effective amount of ESK981, or a pharmaceutically acceptable salt and/or solvate thereof, to a subject in need thereof, wherein:
(a) the subject has NEPC, PDAC, PNETs, NECs, CRC, or NSCLC; and
(b) the NEPC, PDAC, PNETs, NECs, CRC, or NSCLC is characterized as having a KRAS mutation and/or a Trp53 mutation.

In another aspect, the present disclosure provides a method of identifying whether a subject having NEPC, PDAC, PNETs, NECs, CRC, or NSCLC is a candidate for treatment with ESK981, or a pharmaceutically acceptable salt and/or solvate thereof, the method comprising:
(a) determining whether a KRAS mutation and/or a Trp53 mutation is present or absent in a biological sample taken from the subject; and
(b) identifying the subject as being a candidate for treatment if the KRAS mutation and/or a Trp53 mutation is present; or
(c) identifying the subject as not being a candidate for treatment if the KRAS mutation and/or a Trp53 mutation is absent.

In another aspect, the present disclosure provides a method of predicting treatment outcome in a subject having NEPC, PDAC, PNETs, NECs, CRC, or NSCLC, the method comprising determining whether a KRAS mutation and/or a Trp53 mutation is present or absent in a biological sample taken from the subject, wherein:
(a) the presence of a KRAS mutation and/or a Trp53 mutation in the biological sample indicates that administering ESK981, or a pharmaceutically acceptable salt and/or solvate thereof, to the subject will likely cause a favorable therapeutic response; and
(b) the absence of a KRAS mutation and/or a Trp53 mutation in the biological sample indicates that administering ESK981, or a pharmaceutically acceptable salt and/or solvate thereof, to the subject will likely cause an unfavorable therapeutic response.

### BRIEF DESCRIPTION OF DRAWINGS

FIG. 1 is a line graph showing that ESK981 inhibits PIKFyve.

FIG. 2 is a series of four images showing the morphological differences of nuclear-restricted RFP-expressing DU145 cells treated with ESK981 or with siRNA knockdown of PIKfyve.

FIG. 3 is a series of five western blot experiments of prostate cancer cell lines treated with ESK981.

FIG. 4 is a series of four images showing GFP-LC3 levels after treatment of DU145 cells with ESK981.

FIG. 5 is a bar graph showing the number of GFP-LC3 puncta levels in DU145 cells treated with ESK981 over 24 hours.

FIG. 6 is a line graph showing the tumor volume change in VCaP prostate cancer xenograft tumors treated with ESK981.

FIG. 7 is a line graph showing the tumor volume change in DU145 prostate cancer xenograft tumors treated with ESK981.

FIG. 8 is a series of eight images from a terminal deoxynucleotidyl transferase sUTP nick-end labeling (TUNEL) assay of VCaP and DU145 prostate cancer xenografts treated with ESK981.

FIG. 9 is an experimental timeline of Neuroendocrine prostate cancer (NEPC) patient-derived xenograft (PDX) NCI-H660 tumor growth and subsequent treatment with ESK981.

FIG. 10 is a line graph showing the tumor volume change in NEPC PDX NCI-H660 tumors treated with vehicle or ESK981.

FIG. 11 is a bar graph showing the tumor weight change in NEPC PDX NCI-H660 tumors treated with vehicle or ESK981.

FIG. 12 is an experimental timeline of NEPC PDX LTL331R tumor growth and subsequent treatment with ESK981.

FIG. 13 is a line graph showing the tumor volume change in NEPC PDX LTL331R tumors treated with vehicle or ESK981.

FIG. 14 is a bar graph showing the tumor weight change in NEPC PDX LTL331R tumors treated with vehicle or ESK981.

FIG. 15 is an experimental timeline of NEPC PDX LTL610 tumor growth and subsequent treatment with ESK981.

FIG. 16 is a line graph showing the tumor volume change in NEPC PDX LTL610 tumors treated with vehicle or ESK981.

FIG. 17 is a bar graph showing the tumor weight change in NEPC PDX LTL610 tumors treated with vehicle or ESK981.

FIG. 18 is an experimental timeline of NEPC PDX LTL545 tumor growth and subsequent treatment with ESK981.

FIG. 19 is a line graph showing the tumor volume change in NEPC PDX LTL545 tumors treated with vehicle or ESK981.

FIG. 20 is a bar graph showing the tumor weight in NEPC PDX LTL545 tumors treated with vehicle or ESK981.

FIG. 21 is a bar graph showing the tumor volume changes from baseline in NEPC PDX LTL545 tumors treated with vehicle or ESK981.

FIG. 22 is a series of three images showing representative hematoxylin and erosin (H&E) of vehicle or ESK981 treated NEPC PDX LTL545 tumors.

FIG. 23 is a series of four images from a TUNEL assay of vehicle or ESK981 treated NEPC PDX LTL545 tumors.

FIG. 24 is a table of tumor growth inhibition (% TGI) of two AR-positive prostate cancers and six NEPC tumors after ESK981 treatment.

FIG. 25 is a box and whisker plot of % TGI for of two AR-positive prostate cancers and six NEPC tumors after ESK981 treatment.

FIG. 26 is a line graph showing KRAS-mutant PDAC PDX MIA-PaCa-2 tumors treated with vehicle or ESK981 and tumor proliferation after ceasing ESK981 treatment.

FIG. 27 is a bar graph showing the tumor volume changes from baseline in PDAC MIA-PaCa-2 tumors taken from vehicle- or ESK981-treated mice.

FIG. 28 is a series of two immunohistochemistry (IHC) images of Ki67 proliferation maker after treatment with vehicle or ESK981.

FIG. 29 is a series of four images from a TUNEL assay of MIA-PaCa-2 PDAC tumors taken from vehicle- or ESK981-treated mice.

FIG. 30 is a box and whisker plot of pancreatic mass weight of KPC mice (KrasG12D/+; Trp53R172H/+; and p48Cre) treated with vehicle or ESK981.

FIG. 31 is a box and whisker plot of pancreatic mass weight of KC mice (KrasG12D/+; p48Cre) treated with vehicle or ESK981.

FIG. 32 is a box and whisker plot of the percent of KPC mice with lesions after treatment with vehicle of ESK981.

FIG. 33 is a box and whisker plot of the percent of KC mice with lesions after treatment with vehicle of ESK981.

FIG. 34 is a series of two images of example pathology of vehicle and ESK981-treated mice.

FIG. 35 is a box and whisker plot of the pancreatic mass weight of KC mice (KrasG12D/+; p48Cre) and KC mice with one or both Pikfyve alleles deleted.

FIG. 36 is a box and whisker plot of the percent normal KC mice with or without Pikfyve deletion compared to WT.

FIG. 37 is a box and whisker plot of the percent of PaNIN and PDAC lesions in KC mice with or without Pikfyve deletion compared to WT.

FIG. 38 is a line graph of the cell viability of MIA-PaCa-2 cells and MIA-PaCa-2 resistant to trametinib (TramR) or gemcitabine (GemR) treated with TramR.

FIG. 39 is a line graph of the cell viability of MIA-PaCa-2 cells and MIA-PaCa-2 resistant to trametinib (TramR) or gemcitabine (GemR) treated with GemR.

FIG. 40 is a line graph of the cell viability of MIA-PaCa-2 cells and MIA-PaCa-2 resistant to trametinib (TramR) or gemcitabine (GemR) treated with ESK981.

FIG. 41 is a series of six western blot experiments of LC3A/B, MHC-I, and vinculin levels from KPC and MIA-PaCa-2 cells treated with ESK981, apilimod, or bafilomycin.

FIG. 42 is a series of four bar graphs showing the surface MHC-I levels in mouse pancreatic (KPC1361), mammary (4T1), melanoma (B16-F10), and lung (LLC) KPC cancer lines that have been treated with vehicle and ESK981.

FIG. 43 is a series of four bar graphs showing the surface MHC-I levels in mouse pancreatic (KPC1361), mammary (4T1), melanoma (B16-F10), and lung (LLC) KPC cancer lines that have been treated with vehicle and apilimod.

FIG. 44 is a western blot experiment of c-PARP levels in mice with pancreatic neuroendocrine tumors (PNET) PDX QGP-1 tumors treated with vehicle or ESK981.

FIG. 45 is a series of six images showing apoptosis levels from a TUNEL assay of vehicle and ESK981-treated PNET PDX QGP-1 mice.

FIG. 46 is a line graph of tumor volume before and after treatment with vehicle or ESK981 in PNET PDX QGP-1 mice.

FIG. 47A is a box and whisker plot of the In situ Pikfyve levels in KPC murine pancreas lesion vs normal tissue as determined by BaseScope RNA-ISH probes targeting Pikfyve exon 6. (Unpaired two-tailed t-test)

FIG. 47B is a series of six images of a PDAC lesion and normal tissue taken from a KPC murine pancreas, showing H&E, IHC staining for CK19, and BaseScope for Pikfyve.

FIG. 47C is an experimental diagram of the breeding design for the generation of Pikfyve specific deletion in Ptf1a-Cre mice.

FIG. 47D is a series of two western blot experiments of pancreatic tissue from Ptf1a-Cre; encapsulating Pikfyve^{+/+}, Ptf1a-Cre; Pikfyve^{f/+}, and Pikfyve^{f/f} mice in which vinculin was used as a loading control showing changes in PIKfyve protein levels.

FIG. 47E is an experimental diagram of the breeding design for the generation of KC Pikfyve^{+/+}, KC Pikfyve^{f/+}, KC Pikfyve^{f/f} mice.

FIG. 47F is a line graph of the overall survival of KC Pikfyve^{+/+}, KC Pikfyve^{f/+}, KC Pikfyve^{f/f} mice.

FIG. 47G is a box and whisker plot of the pancreas tissue weight normalized to total body weight from KC Pikfyve^{+/+}, KC Pikfyve^{f/+}, KC Pikfyve^{f/f} or age-matched wild-type (WT) mice at 27 weeks of age.

FIG. 47H is a bar graph of the percentage of pancreas occupied by normal tissue as determined by histological analyses in KC Pikfyve^{+/+}, KC Pikfyve^{f/+}, KC Pikfyve^{f/f} mice at 27 weeks of age.

FIG. 47I is a series of sixteen images of representative histological images showing H&E and CK19 staining on pancreatic tissue of KC Pikfyve^{+/+}, KC Pikfyve^{f/+}, and KC Pikfyve^{f/f} mice at 25 weeks of age.

FIG. 47J is an experimental diagram of the breeding design for the generation of KPC Pikfyve^{+/+} and KPC Pikfyve^{f/f} mice.

FIG. 47K is a box and whisker plot of the In situ Pikfyve levels in KPC Pikfyve^{+/+} and KPC Pikfyve^{f/f} murine pancreas lesion versus normal tissue as determined by BaseScope.

FIG. 47L is a bar graph of the pancreatic weight normalized to total body weight from KPC PIKfyve^{+/+} and KPC PIKfyve^{f/f} mice at various ages.

FIG. 47M is a bar graph of the percentage of pancreas occupied by PDAC lesions as determined by histological analyses in KPC PIKfyve^{+/+} and KPC PIKfyve^{f/f} mice throughout experiment duration.

FIG. 47N is a series of six images of representative histology showing CK19 IHC and H&E staining of whole pancreatic tissue from KPC PIKfyve^{+/+} and KPC PIKfyve^{f/f} mice at 25 weeks.

FIG. 48A is a series of two western blot experiments demonstrating stabilization of PIKfyve by apilimod (1000 nM) or ESK981 (1000 nM) in a cellular thermal shift assay (CETSA) employing the murine KPC cell line 7940B.

FIG. 48B is an experimental timeline of the in vivo study assessing the prophylactic efficacy of vehicle or ESK981 (30 mg/kg) on KPC mice.

FIG. 48C is a box and whisker plot of the pancreatic tissue weight in vehicle or ESK981 (30 mg/kg, QD, PO)-treated KPC mice in comparison with age-matched wild-type (WT) mice.

FIG. 48D is a series of four images of representative H&E staining of whole pancreatic tissue from vehicle and ESK981 treated mice (left). On the right is a box and whisker plot of the quantified histologically normal pancreatic tissue in vehicle or ESK981 treated mice. GEMM: genetically engineered mouse model.

FIG. 48E is a series of four images of representative CK19 IHC staining of whole pancreatic tissue from vehicle or ESK981 treated mice (left). On the right is a box and whisker plot of the quantified lesions (PanIN or PDAC) in vehicle or ESK981 treated mice.

FIG. 48F is an experimental timeline of the in vivo efficacy studies utilizing cell-derived xenograft (CDX) or allograft models. Mice were dosed with ESK981 at 30 mg/kg per day (PO) in all studies.

FIG. 48G is a line graph of the tumor volumes of a subcutaneous allograft model using KPC-derived KPC-1344 cells in response to vehicle or ESK981 in C57BL6 mice. Data represented is mean tumor volumes+SEM (n=8 for each cohort).

FIG. 48H is a box and whisker plot of the tumor weights of KPC-1344 model tumors at study endpoint (left). On the right is an image of KPC-1344 model tumors at study endpoint.

FIG. 48I (Left) is a line graph of the tumor volumes of subcutaneous CDX model using MIA PaCa-2 cells in response to vehicle or ESK981 in severe combined immunodeficiency disease (SCID) mice. Data represented is mean tumor volumes+SEM (n=14 for each cohort) (Two-way ANOVA). FIG. 48I (Right) is a bar graph of the changes in tumor volume comparing endpoint to baseline in response to vehicle or ESK981 treatment.

FIG. 48J (Left) is a series of two images of TUNEL staining from primary UM-2 CDX tumors after 5 days of treatment with vehicle or ESK981. FIG. 48J (Right) is a box and whisker plot of the TUNEL positive in UM-2 CDX tumors after 5 days of treatment with vehicle or ESK981. Data represented is from independent tumors and are the mean of 5 representative images per tumor.

FIG. 48K is a series of two western blot experiments of primary UM-2 CDX tumors showing changes in apoptosis marker cleaved PARP (c-PARP) after 5 days treatment of vehicle or ESK981.

FIG. 48L is a line graph of the tumor volumes of a PDAC primary CDX UM-2 model before and after 5 days treatment of vehicle or ESK981.

FIG. 49A is a series of two western blot experiments of MIA PaCa-2 and PANC-1 cells upon CRISPRi-mediated knockdown of PIKfyve with two independent sgRNAs (sgPIKFYVE-1 and sgPIKFYVE-2) or control (sgNC) showing changes in vinculin, loading control, PIKfyve, p62 (SQSTM1), and LC3A/B.

FIG. 49B is a series of two western blot experiments of known autophagy markers vinculin or GAPDH, loading controls, p62 (SQSTM1), and LC3A/B upon treatment with PIKfyve inhibitors apilimod or ESK981 in KPC 7940B and Panc 04.03 cell lines.

FIG. 49C is a box and whisker plot of the tandem fluorescent autophagic flux reporter assay in 7940B cells after 24-hour treatment with apilimod (100 nM), ESK981 (1000 nM), and chloroquine (50 μM) with or without mTORC1/mTORC2 inhibitor torin-1 (100 nM).

FIG. 49D is a line graph of the confluence assay of MIA PaCa-2 cells upon CRISPRi-mediated knockdown of PIKFyve (sgPIKFYVE) or control (sgNC). Data shown are mean+/−SEM (n=4 biological replicates)

FIG. 49E is a box and whisker plot of the IC50s of apilimod, ESK981, and chloroquine in 7 human and mouse PDAC cell lines (specified in FIG. 55B-E). Statistics were performed using a RM one-way ANOVA with Reisser-Greenhouse correction and with Tukey's multiple comparisons test with individual variances computed for each comparison.

FIG. 49F is a gene enrichment rank plot based on differential sgRNA representation in apilimod-treated versus DMSO-treated endpoint populations of the CRISPR screen experiment. Lipid synthesis-related genes ranked at either extreme are highlighted.

FIG. 49G is a scatter plot of the gene fitness scores in apilimod-treated versus DMSO-treated at endpoint conditions in metabolic CRISPR screen. Top 10 hits are labeled, and 5 lipid synthesis-related genes are highlighted.

FIG. 49H is an experimental metabolic diagram of the fatty acid synthesis and elongation, and cholesterol homeostasis. Genes in red indicates a top 10 hit in the CRISPR screen; pink indicates top 90 (3%) hit; light pink indicates the gene was not a top 3% hit; dark grey indicates the gene is universally essential; light grey indicates the gene was not included in the CRISPR screen library.

FIG. 49I is a western blot experiment of MIA PaCa-2 upon CRISPRi-mediated knockdown of FASN in cells. FIG. 49I (Right) is a line graph of the confluence assays assessing the sensitivity of FASN knockdown (sgFASN) or control (sgNC) cells to a vinculin loading control or to apilimod (100 nM). Confluence assay data shown are mean+/−SEM (n=4) from one of two independent experiments. Statistics were performed using an F statistics test based on a two-way ANOVA with Dunnett's multiple comparisons test with the sgFASN+apilimod condition set as a baseline.

FIG. 49J is a western blot experiment assessing the phosphorylation status of ACC1 (p-ACC1) in MIA PaCa-2 cells upon ND646 (ACC inhibitor) treatment with vinculin as a loading control. FIG. 49J (Right) is a line graph of the confluence assays assessing the sensitivity of MIA PaCa-2, PANC-1, and 7940B cells to apilimod, ND646, or both. Concentrations used for apilimod are: 100 nM for MIA PaCa-2, and 50 nM for PANC-1 and 7940B. Concentrations used for ND646 are: 100 nM for MIA PaCa-2 and PANC-1, 1000 nM for 7940B.

FIG. 49K is a heatmap plot of the pathway enrichment analysis of RNA-seq performed on 7940B treated with either apilimod (25 nM) or ESK981 (250 nM) for 8 hours. Dot sizes are inversely proportional to false discovery rate (FDR).

FIG. 49L is a scatter plot of the RNA-seq analysis on 7940B cells treated with apilimod (25 nM) for 8 hours highlighting SREBP-1 target genes. Vertical dashed lines indicate log 2 fold change=+/−0.5. Horizontal dashed line indicates false discovery rate (FDR) of 10-6.

FIG. 49M is a series of three western blot experiments showing PIKfyve, premature SREBP1 (SREBP1 (P)), mature SREBP1 (SREBP1 (M)), and vinculin or histone H3 as loading controls in MIA PaCa-2, PANC-1, and 7940B cells upon treatment with PIKfyve inhibitors for 8 hours. The drug doses used were as follows: for MIA-PaCa-2 and PANC-1: apilimod (300 nM), ESK981 (1000 nM); for 7940B: apilimod (100 nM), ESK981 (1000 nM). “N/A” refers to experimental data not related to the present disclosure.

FIG. 49N is a series of two bar graphs of the quantitative-PCR (qPCR) of MIA PaCa-2 and PANC-1 showing changes in RNA levels of FASN upon CRISPRi-mediated knockdown of PIKfyve.

FIG. 49O is a series of two western blot experiments of MIA PaCa-2 and PANC-1 showing changes in protein levels of vinculin, loading control, or FASN upon CRISPRi-mediated knockdown of PIKfyve.

FIG. 49P is a series of six bar graphs of the qPCR of MIA PaCa-2, PANC-1, and 7940B showing changes in RNA levels of labeled genes upon treatment with PIKfyve inhibitors for 8 hours. The drug doses used were as follows: for MIA PaCa-2 and PANC-1: apilimod (300 nM), ESK981 (1000 nM); for 7940B: apilimod (100 nM), ESK981 (1000 nM).

FIG. 49Q is a series of five western blot experiments of MIA PaCa-2, PANC-1, and 7940B showing changes in protein levels of vinculin, loading control, or labeled genes upon treatment with PIKfyve inhibitors for 24 hours. The drug doses used were indicated on figure or as follows for PANC-1: apilimod (300 nM), ESK981 (1000 nM).

FIG. 49R is a heatmap plot of the glycolytic metabolite abundance in 7940B cells treated with DMSO, apilimod (100 nM), or ESK981 (1000 nM) for 8 hours. * indicates p<0.05, ** indicates p<0.01, *** indicates p<0.0001.

FIG. 49S is a box and whisker forest plot indicating changes in lipid class abundance in 7940B cells upon treatment with DMSO, apilimod (100 nM) or ESK981 (1000 nM) for 24 hours. Effect sizes are in log 2 scale of lipid abundance estimated from separate linear model for each treatment (apilimod or ESK981) compared to DMSO, adjusting for lipid classes with random intercept.

FIG. 49T is a scatter plot of the lipidomics analysis on 7940B cells treated with apilimod for 24 hours, plotting log 2 fold change compared to DMSO highlighted changes in sphingolipid classes (HexCer, SM, Cer). Red highlights indicate upregulated sphingolipids; blue highlights indicate downregulated sphingolipids. Vertical dashed lines indicate log 2 fold change=+/−0.5. Horizontal dashed line indicates p=0.05.

FIG. 50A is a western blot experiment of iKRAS 9805 cells showing changes in protein levels of vinculin, loading control, FASN, and ACC1 upon presence or absence of doxycycline for 72 hours. Phospho-ERK and ERK were used to validate KRAS-MAPK signal inhibition.

FIG. 50B is a series of three western blot experiments of 7940B or PANC-1 cells treated with MEK inhibitor trametinib or KRAS^{G12D} inhibitor MRTX1133 for 48 hours at the indicated concentrations showing changes in protein levels of vinculin, loading control, FASN, and ACACA. Phospho-ERK and ERK were used to validate on-target effects on KRAS-MAPK signaling. MRTX1133 and DMSO were refreshed every 12 hours in experiments involving MRTX1133.

FIG. 50C is a bar graph of the qPCR of iKRAS 9805 cells showing changes in mRNA levels of Fasn upon 48-hour incubation with or without doxycycline and subsequent 8-hour treatment with apilimod (50 nM), ESK981 (300 nM), or DMSO.

FIG. 50D is a western blot experiment of iKRAS 9805 cells showing changes in protein levels of vinculin, loading control, FASN, ACC1, p62, and LC3A/B upon 48 hour incubation with or without doxycycline (Dox) and subsequent 24-hour treatment with apilimod (50 nM), ESK981 (300 nM), or DMSO. p-ERK and ERK were assessed to validate KRAS OFF.

FIG. 50E is a box and whisker plot of the tandem fluorescent reporter assay on iKRAS 9805 cells showing changes in the autophagic flux after 24 hours withdrawal from doxycycline and subsequent treatment with apilimod (100 nM), ESK981 (1000 nM) or chloroquine (10 μM) for 24 hours.

FIG. 50F is a series of two box and whisker plots of the tandem fluorescent reporter assay on 7940B cells showing changes in the autophagic flux upon 4-hour pretreatment with apilimod (100 nM), ESK981 (1000 nM) or chloroquine (50 μM) and subsequent treatment with MRTX1133 (300 nM) or trametinib (25 nM) for 24 hours.

FIG. 50G (Left) is a 3D synergy map and FIG. 50G (Right) is a heatmap plot of 7940B cells treated with apilimod and trametinib. Red peaks in the 3D synergy map indicates synergism, and the overall average synergy score is listed above. The heatmap plots the decrease in viability in 7940B upon treatment with each single agent or combination across indicated doses of each inhibitor.

FIG. 50H is a line graph of the confluence assay in PANC-1 cells treated with apilimod (50 nM) and/or MRTX1133 (300 nM).

FIG. 50I is an experimental timeline outlining the syngeneic orthotopic model of 7940B for C57BL/6 mice assessing in vivo efficacy of ESK981 (30 mg/kg, QD, PO), trametinib (1 mg/kg QD, PO), or ESK981 and trametinib.

FIG. 50J is a box and whisker plot of the endpoint pancreas+tumor weight normalized to total body weight. Pancreata of 6 age-matched non-tumor bearing C57BL/6 mice were used as references.

FIG. 50K is a box and whisker plot of the quantification of proportion of PDAC in H&E sections from each tumor of the 7940B syngeneic orthotopic model.

FIG. 50L is a box and whisker plot of the quantification of CK19 positive area compared to hematoxylin counterstain on a section from each tumor of the 7940B syngeneic orthotopic model.

FIG. 50M is a series of six representative images of H&E and CK19 IHC staining of one tumor from each treatment pathway of the 7940B syngeneic orthotopic model.

FIG. 50N is an experimental timeline outlining the efficacy study using a subcutaneous model of UM-19 primary cell-derived xenograft (CDX) treated with vehicle, MRTX1133 (30 mg/kg, QD, IP), ESK981 (30 mg/kg, QD, PO), or ESK981+MRTX1133.

FIG. 50O is a line graph of the tumor volumes as a percentage+SEM of the initial volume measured by calipers of the UM-19 primary CDX (pCDX) model treated with MRTX1133 or in combination with ESK981.

FIG. 50P is a bar graph of the change in tumor volume at treatment end point (day 19) compared to baseline of the UM-19 pCDX model treated with MRTX1133 or in combination with ESK981.

FIG. 50Q is an experimental timeline outlining the efficacy study using a subcutaneous model of UM-19 pCDX treated with vehicle, trametinib (1 mg/kg, QD, PO), ESK981 (30 mg/kg, QD, PO), or ESK981+trametinib.

FIG. 50R is a line graph of the tumor volumes as a percentage+SEM of the initial volume measured by calipers of the UM-19 pCDX model treated with trametinib or in combination with ESK981. The tumors in the vehicle- and ESK981-treated groups were the same tumors shown in FIG. 50O.

FIG. 50S is a bar graph of the change in tumor volume at treatment end point compared to baseline of the UM-19 CDX model treated with trametinib or in combination with ESK981. The endpoint displayed for the vehicle and trametinib pathways are day 19. The endpoint displayed of the ESK981 and ESK981+trametinib pathways are day 57. The tumors in the vehicle- and ESK981-treated groups were the same tumors shown in FIG. 50O.

FIG. 50T is a line graph of the Kaplan-Meier estimates of time to tumor doubling at treatment end point compared to baseline of the UM-19 CDX model treated with trametinib or in combination with ESK981. The endpoint displayed for the vehicle and trametinib pathways are day 19. The endpoint displayed of the ESK981 and ESK981+trametinib pathways are day 57.

FIG. 51 is a schematic diagram of PDAC Metabolic Homeostasis, in functional PIKfyve and KRAS-MAPK signaling (Top Left), PDAC is at metabolic homeostasis, able to generate lipids both through de novo synthesis as well as through lysosomal processes. Upon PIKfyve inhibition, autophagy and lysosomal functions are disrupted, forcing PDAC to upregulate and depend on de novo fatty acid synthesis through FASN and ACC1 (Top Right). KRAS-MAPK inhibition decreases expression of FASN and ACC1 and increases PDAC cells' dependence on autophagy and lysosomal processes (Bottom Left). Concurrent PIKfyve and KRAS-MAPK inhibition results in lethal metabolic crises in PDAC (Bottom Right).

FIG. 52A is a bar graph of the densitometry analyses of immunoblot displayed in FIG. 47D.

FIG. 52B is a box and whisker plot of the pancreas tissue weight normalized to total body weight for Ptf1a-Cre; Pikfyve^{+/+}, Ptf1a-Cre; Pikfyve^{f/+}, and Ptf1a-Cre; Pikfyve^{f/f} mice.

FIG. 52C is a series of six representative images of H&E and insulin IHC staining from the pancreas tissue of Ptf1a-Cre; Pikfyve^{+/+}, Ptf1a-Cre; Pikfyve^{f/+}, and Ptf1a-Cre; Pikfyve^{f/f} mice.

FIG. 52D is a box and whisker plot of the PIKfyve levels as determined by BaseScope of KC Pikfyve^{+/+}, KC Pikfyve^{f/+}, and KC Pikfyve^{f/f} murine pancreas tissue separated by normal and lesional areas.

FIG. 52E is a series of six representative images of PIKfyve Basescope staining from pancreas tissue of 27-week-old KC Pikfyve^{+/+}, KC Pikfyve^{f/+}, and KC Pikfyve^{f/f} mice.

FIG. 52F is a box and whisker plot of the pancreas tissue weight from 27-week-old KC Pikfyve^{+/+}, KC Pikfyve^{f/+}, KC Pikfyve^{f/f}, and age-matched wild-type (WT) mice.

FIG. 52G is a series of two box and whisker plots of the pancreas tissue weight normalized to total body weight (left) and raw pancreas tissue weight (right) from 40-week old KC Pikfyve^{+/+}, KC Pikfyve^{f/+}, and KC Pikfyve^{f/f}, and age-matched wild-type (WT) mice.

FIG. 52H is a bar graph of the percentage of pancreata occupied by normal tissue as determined by histological analyses of KC Pikfyve^{+/+}, KC Pikfyve^{f/+}, and KC Pikfyve^{f/f} mice at 40 weeks of age.

FIG. 52I is a series of two representative images of PIKfyve Basescope staining from pancreas tissue of 25-week-old KPC PIKfyve^{+/+} and KPC PIKfyve^{f/f} mice.

FIG. 53A is a line graph of the tumor volumes of subcutaneous CDX model derived from BxPC3 cells in response to vehicle or ESK981 in SCID mice. Data plotted are mean tumor volumes+SEM (n=10 for each cohort).

FIG. 53B (Left) is a box and whisker plot of the individual weights and FIG. 53B (Right) is an image of tumors from CDX model derived from BxPC3 cells at endpoint.

FIG. 53C is a line graph of the Kaplan-Meier estimates of time to tumor tripling of BxPC3 CDX tumors after vehicle or ESK981 treatment.

FIG. 53D is a series of eight representative images of H&E and Ki67 IHC staining in MIA-PaCa-2 and BxPC3 CDX models post vehicle or ESK981 treatment.

FIG. 53E is a series of six representative images of DAPI staining, TUNEL staining, or merged from MDA-PaCa-2 CDX tumors after 5 days of treatment of vehicle or ESK981.

FIG. 53F is a line graph of the tumor volumes of subcutaneous CDX model derived from T24 cells in response to vehicle or ESK981 in SCID mice.

FIG. 53G (Top) is a box and whisker plot of the individual weights and FIG. 53G (Bottom) is an image of tumors from CDX model derived from T24 cells at endpoint.

FIG. 54A is a series of two bar graphs of the qPCR of MIA PaCa-2 or PANC-1 cells upon CRISPRi-mediated knockdown of PIKfyve with two independent sgRNAs (sgPIKFYVE-1 and sgPIKFYVE-2) validating target knockdown compared to control (sgNC).

FIG. 54B is a western blot experiment of UM-2 primary cell-derived xenograft tumors after 5 days of treatment with either vehicle or ESK981 (30 mg/kg) as described in FIG. 48J showing changes in GAPDH, loading control, or LC3A/B.

FIG. 54C is a box and whisker plot of the tandem fluorescent reporter assay in Panc 04.03 cells showing changes in autophagic flux upon 4-hour pre-treatment with DMSO, apilimod (300 nM), ESK981 (1000 nM), or chloroquine (30 mM) and subsequent treatment of torin-1 (100 nM) or DMSO for 24 hours.

FIG. 54D is a series of four images of MIA-PaCa-2 and PANC-1 cells upon CRISPRi-mediated knockdown of PIKfyve or sgNC control.

FIG. 54E is a series of eight images of 7940B and MIA-PaCa-2 cells upon treatment with PIKfyve inhibitors apilimod or ESK981 for 4 hours.

FIG. 54F is 20× bright-field images of MIA-PaCa-2 (left) and PANC-1 (right) cells upon CRISPRi-mediated knockdown of PIKFYVE or control.

FIG. 54G is G. 20× bright-field images of 7940B (top) MIA-PaCa-2 (bottom) cells upon treatment with PIKfyve inhibitors apilimod or ESK981 or PIKfyve degrader PIK5-33d for 4 hours.

FIG. 55A is a line graph of the confluence assay of PANC-1 cells upon CRISPRi-mediated knockdown of PIKfyve (sgPIKFYVE-1 and sgPIKFYVE-2) or control (sgNC).

FIG. 55B is a line graph of the dose-response curve series of indicated PDAC cell lines treated with apilimod, for 7 days using Cell-TiterGlo assays.

FIG. 55C is a line graph of the dose-response curve series of indicated PDAC cell lines treated with ESK981 for 7 days using Cell-TiterGlo assays.

FIG. 55D is a line graph of the dose-response curve series of indicated PDAC cell lines treated with chloroquine for 7 days using Cell-TiterGlo assays.

FIG. 55E is a table of IC₅₀ values of each drug in each cell line tested.

FIG. 56A is a western blot experiment of 7940B cells treated with apilimod or ESK981 at indicated timepoints assessing changes in vinculin, loading control. HIF1α, p62, and LC3A/B.

FIG. 56B is a series of two line graphs of the oxygen consumption rate (OCR) in 7940B and Panc 04.03 cells upon treatment with apilimod (100 nM), ESK981 (1000 nM), bafilomycin (100 nM), or chloroquine (100 μM) for 8 hours.

FIG. 56C (Left) is a line graph of the real-time oxygen consumption rate monitoring by Resipher on 7940B cells upon treatment with apilimod (100 nM), ESK981 (1000 nM), bafilomycin (100 nM), or chloroquine (100 μM). FIG. 55C (Right) is a series of two bar graphs of the oxygen consumption rate at 8 hours and 24 hours by Resipher measurements from the same experiment.

FIG. 56D is a series of eight line graphs of the dose-response curve of 7940B and PANC-1 cells treated with bafilomycin, apilimod, ESK981, or chloroquine in the presence or absence of ferric ammonium citrate and ferrostatin-1 (1 μM).

FIG. 56E is a series of two tables of IC₅₀ values of bafilomycin, apilimod, ESK981, or chloroquine) treatment in 7940B and PANC-1 cell lines with or without ferric ammonium citrate co-treatment.

FIG. 56F is a series of two line graphs of the confluence assay of Panc 04.03 cells undergoing treatment with DFO (100 μM), apilimod (300 nM), ESK981 (1000 nM), chloroquine (100 μM), bafilomycin (100 nM), without or with ferric ammonium citrate (100 g/mL) and ferrostatin-1 (1 μM).

FIG. 57A is an experimental diagram of the metabolism-focused CRISPR screen in MIA PaCa-2 cells.

FIG. 57B is a series of two line graphs of the receiver operator characteristic (ROC) curves for the prediction of core essential genes using datasets from MIA PaCa-2 CRISPR screens.

FIG. 57C is a bar graph of the qPCR experiment showing changes in mRNA levels of FASN upon CRISPRi-mediated knockdown of FASN in MIA PaCa-2 cells.

FIG. 57D is a series of two western blot experiments of PANC-1 and 7940B cells upon treatment with ND646 at indicated doses for 24 hours showing changes in treatment with vinculin, loading control, phosphorylation status of ACC1 (p-ACC1), and ACC1.

FIG. 57E is a series of three line graphs of the confluence assays of MIA PaCa-2, PANC-1, and 7940B cells upon treatment with ND646 (100 nM for MIA-PaCa2 and PANC-1, 1000 nM for 7940B) and ESK981 (30 nM for MIA PaCa-2, 100 nM for PANC-1 and 7940B).

FIG. 57F shows confluence assays of MIA PaCa-2, PANC-1, and 7940B cells upon treatment with ND646 (100 nM for MIA-PaCa2 and PANC-1, 1000 nM for 7940B) and ESK981 (30 nM for MIA PaCa-2, 100 nM for PANC-1 and 7940B). Data shown are mean+/−SEM from 4 biological replicates. p-values were performed using a two-way ANOVA with Dunnett's multiple comparisons test with the combination condition as the comparison group.

FIG. 57G shows confluence assays of MIA PaCa-2, PANC-1, and 7940B cells upon treatment with ND646 (100 nM for MIA-PaCa2 and PANC-1, 1000 nM for 7940B) and PIK5-33d (100 nM for MIA PaCa-2, 1000 nM for PANC-1 and 7940B). Data shown are mean+/−SEM from 4 biological replicates. p-values were determined using a two-way ANOVA with Dunnett's multiple comparisons test with the combination condition as the comparison group.

FIG. 58A is a scatter plot of the log 2 fold change in gene expression upon 8-hour treatment with apilimod (100 nM) vs DMSO control (x-axis) and ESK981 (1000 nM) vs DMSO control (y-axis).

FIG. 58B is a series of four GSEA line graphs of cholesterol homeostasis and fatty acid metabolism using the fold change rank-ordered gene signature from the 7940B cells treated with apilimod or ESK981 for 8 hours.

FIG. 58C is a scatter plot of the RNA-seq analysis on 7940B cells treated with ESK981 (1000 nM) for 8 hours highlighting SREBP-1 target genes.

FIG. 59A is a scatter plot of the principal component analysis (PCA) of targeted metabolomics experiment on 7940B cells treated with DMSO, apilimod (100 nM), or ESK981 (1000 nM) for 8 hours.

FIG. 59B is a series of two bar graph of the citrate levels as detected by LC-MS-based metabolomics on 7940B cells treated with apilimod (100 nM) or ESK981 (1000 nM) for either 3 or 8 hours, as indicated.

FIG. 59C is a heatmap of the metabolites showing significant changes as determined by unpaired two-tailed t-test (apilimod vs DMSO or ESK981 vs DMSO, p<0.05 in at least one of the two comparisons).

FIG. 59D is a scatter plot of the principal component analysis (PCA) of targeted lipidomics experiment on 7940B cells treated with DMSO, apilimod (100 nM), or ESK981 (1000 nM) for 24 hours.

FIG. 59E is a scatter plot of the abundant lipid species in 7940B cells upon treatment with ESK981 (1000 nM) for 24 hours. Lipids differentially highlighted in red are upregulated sphingolipid species while Lipids differentially highlighted in blue are downregulated sphingolipid species.

FIG. 60A is a series of two bar graph of the qPCR of iKRAS (doxycycline-inducible KRAS^{G12D}) 9805 cells showing changes in RNA levels of labeled genes in the presence or absence of doxycycline for 48 hours.

FIG. 60B is a series of six bar graph of the qPCR of 7940B, PANC-1, and MIA PaCa-2 cells treated with KRAS^{G12D} inhibitor MRTX1133 (100 nM for 7940B, 1000 nM for PANC-1), KRAS^{G12C} inhibitor AMG510 (100 nM for MIA PaCa-2), or MEK inhibitor trametinib (10 nM for MIA PaCa-2, 30 nM for 7940B, 100 nM for PANC-1) for 8 hours.

FIG. 60C is a series of two bar graph of the counts per million (CPM) from RNA-seq analysis on AsPC1 cells treated with MRTX1133 (100 nM) for 24 hours Hallin, J. et al., Nat. Med. 2022:28: 2171-2182.

FIG. 60D is a series of two bar graph of the counts per million (CPM) from RNA-seq analysis on AsPC1 cell-derived xenograft model Hallin, J. et al., Nat. Med. 2022:28: 2171-2182. Mice were dosed with 30 mg/kg of MRTX1133 6 hours prior to tumor collection.

FIG. 60E is a series of two box and whisker plots of the tandem fluorescent reporter assay in 7940B or Panc 04.03 cells showing changes in autophagic flux upon 24-hour treatment with labeled doses of MRTX1133.

FIG. 60F is a series of two box and whisker plot of the tandem fluorescent reporter assay in Panc 04.03 cells showing changes in autophagic flux upon 4-hour pretreatment with apilimod (300 nM), ESK981 (1000 nM), or chloroquine (30 mM) followed by 24-hour treatment with MRTX1133 (300 nM or trametinib (25 nM).

FIG. 61A is a series of three 3D synergy maps of 7940B cells treated with ESK981 and trametinib, apilimod and MRTX1133, and ESK981 and MRTX1133. Red peaks in the 3D synergy maps indicates synergism, and the overall average synergy score is listed above.

FIG. 61B is a series of three heatmap plots of 7940B cells treated with ESK981 and trametinib, apilimod and MRTX1133, and ESK981 and MRTX1133. Values in heatmap indicate mean relative decrease in viability compared to DMSO.

FIG. 61C is a line graph of the confluence assays of 7940B cells treated with DMSO, trametinib (20 nM), apilimod (50 nM), or both trametinib and apilimod.

FIG. 61D is a line graph of the confluence assays of PANC-1 cells treated with DMSO, MRTX1133 (300 nM), ESK981 (100 nM), or both MRTX1133 and ESK981.

FIG. 62A is a line graph of the relative body weight (compared to day 1) of mice bearing 7940B orthotopic tumors undergoing trametinib, ESK981, or trametinib+ESK981 treatment.

FIG. 62B is a box and whisker plot of the raw pancreas+tumor weights collected at endpoint of 7940B syngeneic orthotopic model undergoing trametinib, ESK981, or trametinib+ESK981 treatment.

FIG. 62C is a bar graph of the tumor presence or absence based on histological evidence of the 7940B syngeneic orthotopic model undergoing trametinib, ESK981, or trametinib+ESK981 treatment.

### DETAILED DESCRIPTION

It has been unexpectedly discovered that PIKfyve, a lipid kinase integral to lysosomal functioning, is a targetable vulnerability in PDAC. Employing a genetically engineered mouse model, the essential role of PIKfyve in PDAC progression was established. Also, through comprehensive metabolic analyses, it was found that PIKfyve inhibition obligates PDAC to upregulate de novo lipid synthesis. PIKfyve inhibition triggers a distinct lipogenic gene expression and metabolic program, creating a dependency on de novo lipid metabolism pathways, including genes such as FASN and ACACA. In PDAC, the KRAS-MAPK signaling pathway is a primary driver of de novo lipid synthesis, specifically enhancing FASN and ACACA levels. By inhibiting the KRAS-MAPK pathway, a synthetic lethality in PDAC that follows PIKfyve inhibition can be exploited. To this end, the simultaneous targeting of PIKfyve and KRAS-MAPK resulted in the elimination of tumor burden in a syngeneic orthotopic model and surprising tumor elimination in PDAC xenograft models. Taken together, these studies suggest that disrupting lipid metabolism through PIKfyve inhibition induces synthetic lethality in conjunction with KRAS-MAPK directed therapies for PDAC.

In one embodiment, the present disclosure provides methods of treating cancer, e.g., prostate cancer, pancreatic cancer, colorectal cancer, or non-small cell lung cancer, in a subject in need thereof, the method comprising administering to the subject a therapeutically effective amount of ESK981, or a pharmaceutically acceptable salt and/or solvate thereof.

In another embodiment, a therapeutically effective amount of an ESK981 Solid Form is administered to the subject.

In another embodiment, a therapeutically effective amount of an ESK981 Polymorph is administered to the subject.

In another embodiment, the present disclosure provides methods of treating cancer, e.g., prostate cancer, pancreatic cancer, colorectal cancer, or non-small cell lung cancer, in a subject in need thereof, the method comprising administering to the subject a therapeutically effective amount of a PIKfyve inhibitor, e.g., ESK981, in combination with a therapeutically effective amount of a KRAS-MAPK inhibitor. Exemplary non-limiting KRAS-MAPK inhibitors include AMG510 (Sotorasib), MRTX849 (Adagrasib), and MRTX-1133. Without wishing to be bound by any particular theory, the combination of PIKfyve inhibition and KRAS-MAPK inhibition causes synthetic lethality to the cancer cells. In another embodiment, the prostate cancer is NEPC.

In another embodiment, the pancreatic cancer is PDAC, PNETs, or NECs.

In another embodiment, the prostate cancer or pancreatic cancer is characterized as having a KRAS mutation and/or a Trp53 mutation.

In another embodiment, the present disclosure provides methods of treating cancer, e. g., non-small cell lung cancer (NSCLC), prostate cancer, e.g. neuroendocrine prostate cancer (NEPC), pancreatic cancer, e.g., pancreatic ductal adenocarcinoma (PDAC), pancreatic neuroendocrine tumors (PNETs), or pancreatic neuroendocrine carcinomas (NECs), or colorectal cancer (CRC), in a subject in need thereof, the method comprising administering to the subject a therapeutically effective amount of a PIKfyve inhibitor, e.g., ESK981, or a pharmaceutically acceptable salt and/or solvate thereof, in combination with a therapeutically effective amount of a RAS inhibitor.

In another embodiment, a therapeutically effective amount of an ESK981 Solid Form is administered to the subject.

In another embodiment, a therapeutically effective amount of an ESK981 Polymorph is administered to the subject.

In another embodiment, the cancer is NSCLC.

In another embodiment, the cancer is prostate cancer.

In another embodiment, the prostate cancer is NEPC.

In another embodiment, the cancer is pancreatic cancer.

In another embodiment, the pancreatic cancer is PDAC, PNETs, or NECs.

In another embodiment, the cancer is CRC.

In another embodiment, the cancer, e.g., NSCLC, prostate cancer, pancreatic cancer, or colorectal cancer, is characterized as having a KRAS mutation.

In another embodiment, the KRAS mutation is a KRAS^{G12C} mutation.

In another embodiment, the KRAS mutation is a KRAS^{G12D} mutation.

In another embodiment, the KRAS mutation is a KRAS^{G12V} mutation.

In another embodiment, the KRAS mutation is a KRAS^{G13C} mutation.

In another embodiment, the KRAS mutation is a KRAS^{G13C} mutation.

In another embodiment, the KRAS mutation is a KRAS^{Q61H} mutation.

In another embodiment, the ESK981 Polymorph is ESK981 pTSA-A₁.

In another embodiment, the ESK981 Polymorph is ESK981 pTSA-A₂.

In another embodiment, the ESK981 Polymorph is ESK981 pTSA-B₂.

In another embodiment, the ESK981 Polymorph is ESK981 pTSA-C₂.

In another embodiment, the ESK981 Polymorph is ESK981 AA.

In another embodiment, the RAS inhibitor is a pan-RAS inhibitor.

In another embodiment, the RAS inhibitor is a RAS^{WT/X} inhibitor.

In another embodiment, the RAS inhibitor is a KRAS inhibitor.

In another embodiment, the KRAS inhibitor is a KRAS^{G12C} inhibitor.

In another embodiment, the KRAS inhibitor is a KRAS^{G12D} inhibitor.

In another embodiment, the KRAS inhibitor is a KRAS^{G12V} inhibitor.

In another embodiment, the KRAS inhibitor is a KRAS^{G13C} inhibitor.

In another embodiment, the KRAS inhibitor is a KRAS^{Q61H} inhibitor.

In another embodiment, the RAS inhibitor is a compound listed in Table 2A.

TABLE 2A

| | Common | |
|---|---|---|
| Code Name | Name | Chemical Structure |
| MRTX849 | Adagrasib | 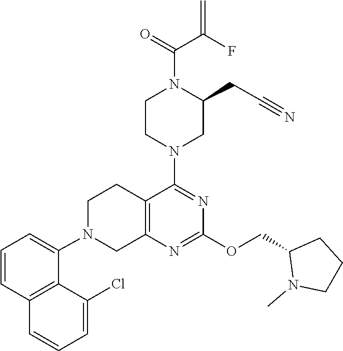 [SMILES: C=C(F)C(=O)N1CCN(c2nc(OC[C@@H]3CCCN3C)nc3c2CCN(c2cccc4cccc(Cl)c24)C3)C[C@@H]1CC#N] |
| AMG 510 | Sotorasib | 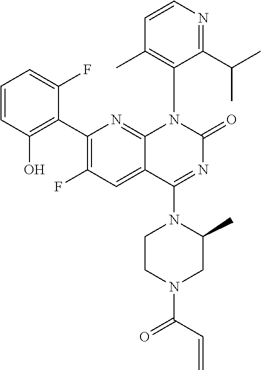 [SMILES: C=CC(=O)N1CCN(c2nc(=O)n(-c3c(C)ccnc3C(C)C)c3nc(-c4c(O)cccc4F)c(F)cc23)[C@@H](C)C1] |
| BBO-8520 | | 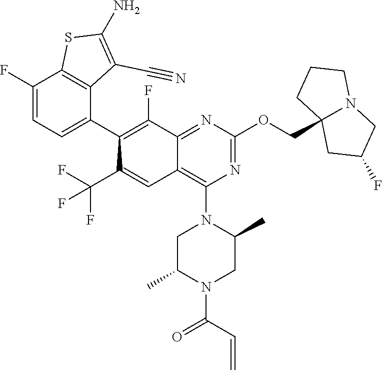 |
| BBO-8956 | | 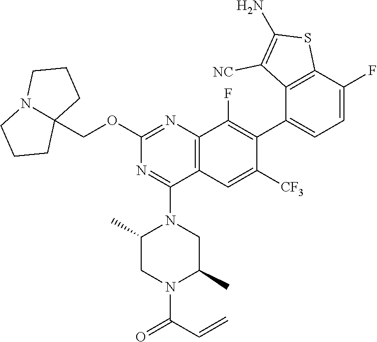 [SMILES: C=CC(=O)N1C[C@H](C)N(c2nc(OCC34CCCN3CCC4)nc3c(F)c(-c4ccc(F)c5sc(N)c(C#N)c45)c(C(F)(F)F)cc23)C[C@H]1C] |
| D3S-001 | Elisrasib | 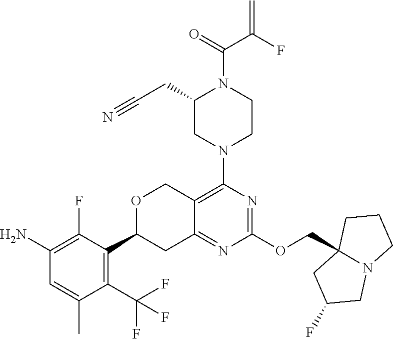 [SMILES: C=C(F)C(=O)N1CCN(c2nc(OC[C@@]34CCCN3C[C@H](F)C4)nc3c2CO[C@H](c2c(F)c(N)cc(C)c2C(F)(F)F)C3)C[C@@H]1CC#N] |
| GDC-6036 | Divarasib | 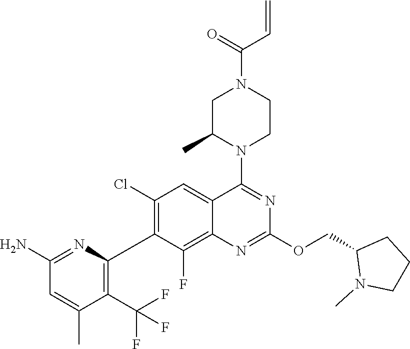 |
| D-1553 | Garsorasib | 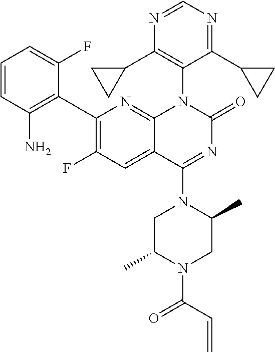 [SMILES: C=CC(=O)N1C[C@H](C)N(c2nc(=O)n(-c3c(C4CC4)ncnc3C3CC3)c3nc(-c4c(N)cccc4F)c(F)cc23)C[C@H]1C] |
| GH35 | | 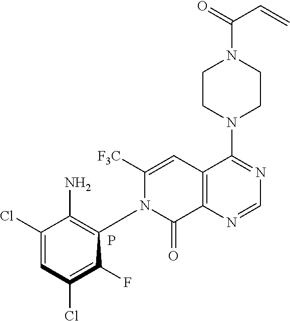 |
| JAB-21822 | Glecirasib | 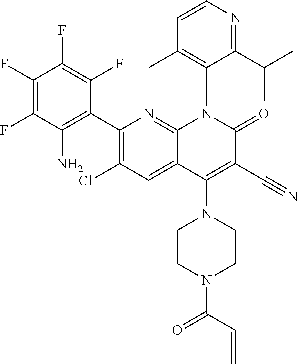 [SMILES: C=CC(=O)N1CCN(c2c(C#N)c(=O)n(-c3c(C)ccnc3C(C)C)c3nc(-c4c(N)c(F)c(F)c(F)c4F)c(Cl)cc23)CC1] |
| IBI351/GFH925 | Fulzerasib | 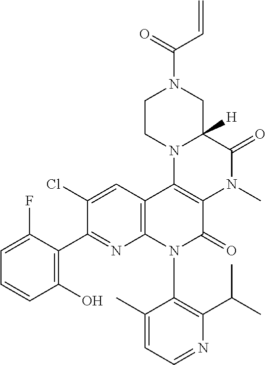 [SMILES: C=CC(=O)N1CCN2c3c(c(=O)n(-c4c(C)ccnc4C(C)C)c4nc(-c5c(O)cccc5F)c(Cl)cc34)N(C)C(=O)[C@H]2C1] |
| LY3499446 | | 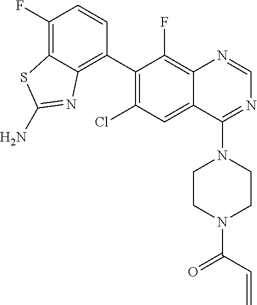 [SMILES: C=CC(=O)N1CCN(c2ncnc3c(F)c(-c4ccc(F)c5sc(N)nc45)c(Cl)cc23)CC1] |
| MK-1084 | | 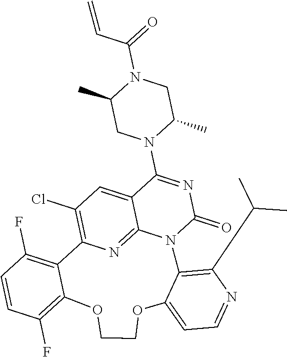 [SMILES: C=CC(=O)N1C[C@H](C)N(c2nc(=O)n3c4nc(c(Cl)cc24)-c2c(F)ccc(F)c2OCCOc2ccnc(C(C)C)c2-3)C[C@H]1C] |
| LY3537982 | Olomorasib | 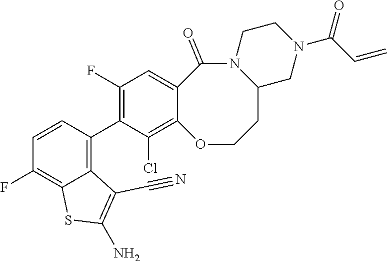 [SMILES: C=CC(=O)N1CCN2C(=O)c3cc(F)c(-c4ccc(F)c5sc(N)c(C#N)c45)c(Cl)c3OCCC2C1] |
| JDQ443 | Opnurasib | 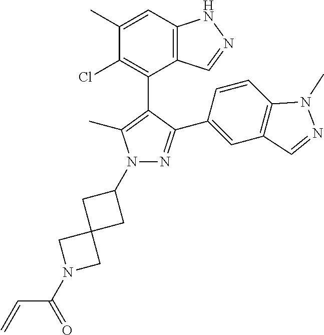 [SMILES: C=CC(=O)N1CC2(CC(n3nc(-c4ccc5c(cnn5C)c4)c(-c4c(Cl)c(C)cc5[nH]ncc45)c3C)C2)C1] |
| RMC-4998 | | 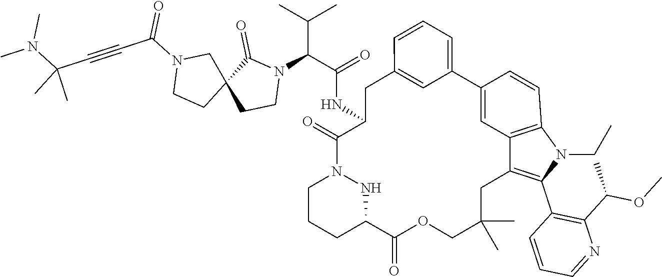 |
| RMC-6291 | Elironrasib | 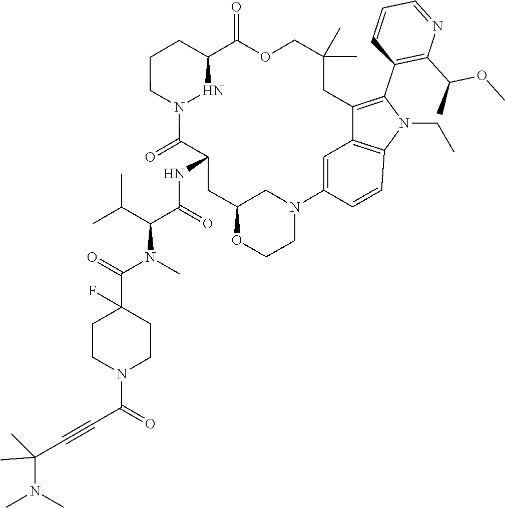 |
| ASP3082 | | 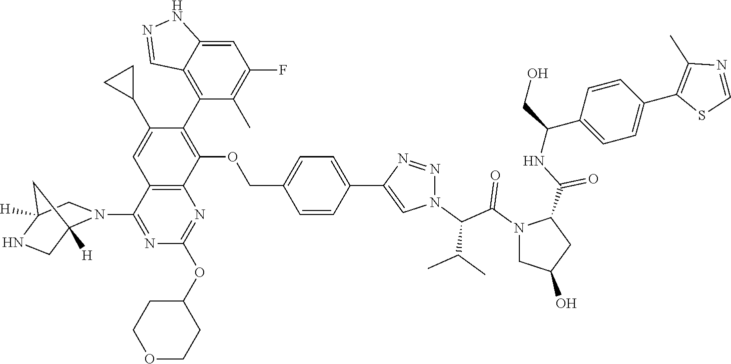 [SMILES: Cc1ncsc1-c1ccc([C@H](CO)NC(=O)[C@@H]2C[C@@H](O)CN2C(=O)[C@H](C(C)C)n2cc(-c3ccc(COc4c(-c5c(C)c(F)cc6[nH]ncc56)c(C5CC5)cc5c(N6C[C@@H]7C[C@H]6CN7)nc(OC6CCOCC6)nc45)cc3)nn2)cc1] |
| HRS-4642 | | 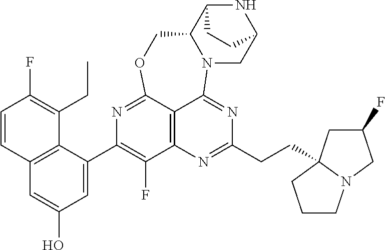 [SMILES: CCc1c(F)ccc2cc(O)cc(-c3nc4c5c(nc(CC[C@@]67CCCN6C[C@H](F)C7)nc5c3F)N3C[C@H]5CC[C@H](N5)[C@H]3CO4)c12] |
| MRTX1133 | | 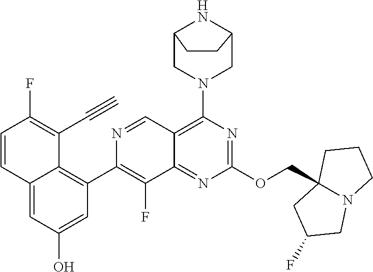 [SMILES: C#Cc1c(F)ccc2cc(O)cc(-c3ncc4c(N5CC6CCC(C5)N6)nc(OC[C@@]56CCCN5C[C@H](F)C6)nc4c3F)c12] |
| RMC-9805 | Zoldonrasib | 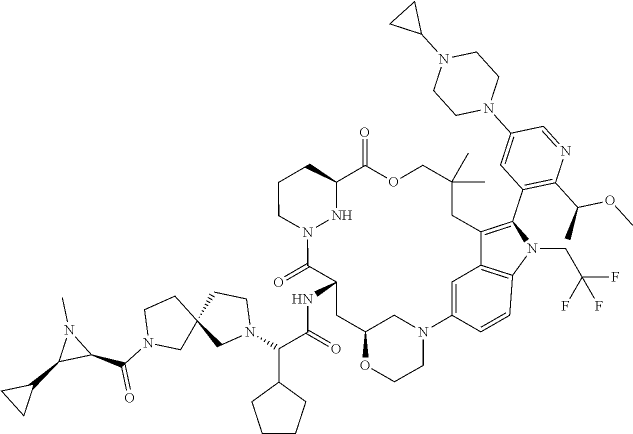 |
| RMC-5127 | | 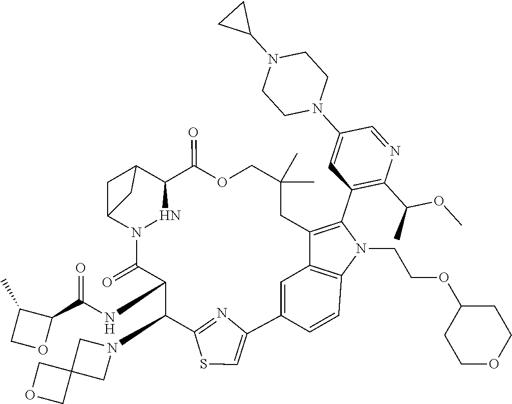 |
| ACBI3 | | 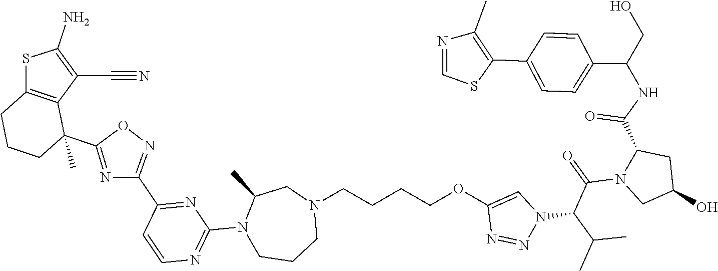 [SMILES: Cc1ncsc1-c1ccc(C(CO)NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)cc1] |
| BI-2493 | | 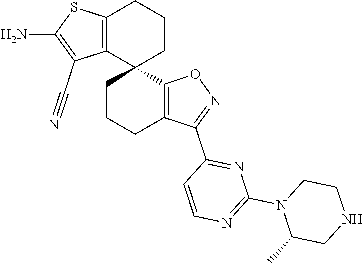 [SMILES: C[C@H]1CNCCN1c1nccc(-c2noc3c2CCC[C@@]32CCCc3sc(N)c(C#N)c32)n1] |
| BI-2865 | | 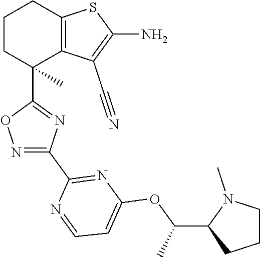 [SMILES: C[C@H](Oc1ccnc(-c2noc([C@@]3(C)CCCc4sc(N)c(C#N)c43)n2)n1)[C@@H]1CCCN1C] |
| RMC-6236* | Daraxonrasib | 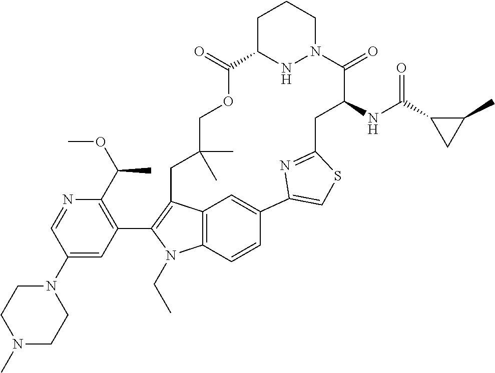 [SMILES: CCn1c(-c2cc(N3CCN(C)CC3)cnc2[C@H](C)OC)c2c3cc(ccc31)-c1csc(n1)C[C@H](NC(=O)[C@H]1C[C@@H]1C)C(=O)N1CCC[C@H](N1)C(=O)OCC(C)(C)C2] |
| RMC-7977* | | 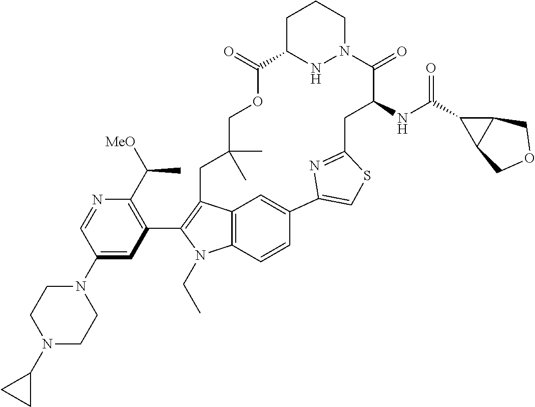 |

In another embodiment, the RAS inhibitor is a compound listed in Table 2B.

TABLE 2B

BEBT-607
BI 1823911
BPI-421286
FMC-376
GEC255
HBI-2438
HS-10370
JNJ-74699157
YL-15293
GFH357/VS-7375
INCB161734
JAB-22000
LY3962673
QTX3046
TSN1611
RMC-8839
RMC-0708
BI 3706674
JAB-23E73
LY4066434
PF-07934040
QTX3034
GFH547
YL-17231
BPI-585725
ERAS-4001

In another embodiment, the RAS inhibitor is a compound listed in Table 2A and Table 2B.

In another embodiment, the RAS inhibitor is disclosed in U.S. 20240262847. In another embodiment, the RAS inhibitor is disclosed as Formula I0, Formula II0, Formula III0, or Formula IV0 in claim 1 of U.S. 20240262847. In another embodiment, the RAS inhibitor is a species disclosed in Table 1 of U.S. 20240262847.

In another embodiment, the RAS inhibitor is disclosed in U.S. 20240408064. In another embodiment, the RAS inhibitor is disclosed as Formula I in claim 1 of U.S. 20240408064. In another embodiment, the RAS inhibitor is a species disclosed in one of Tables 1-20 of U.S. 20240408064.

In another embodiment, the RAS inhibitor is disclosed in U.S. 20250084079. In another embodiment, the RAS inhibitor is disclosed as Formula I in claim 1 of U.S. 20250084079. In another embodiment, the RAS inhibitor is a species disclosed in Tables 2 or 3 of U.S. 20250084079.

In another embodiment, the RAS inhibitor is disclosed in U.S. 20250186590. In another embodiment, the RAS inhibitor is disclosed as Formula Ia in U.S. 20250186590. In another embodiment, the RAS inhibitor is a species disclosed in Tables 1a, 1b, 2, or 3 of U.S. 20250186590.

In another embodiment, the RAS inhibitor is disclosed in U.S. 20250188085. In another embodiment, the RAS inhibitor is disclosed as Formula I in claim 1 of U.S. 20250188085. In another embodiment, the RAS inhibitor is a species disclosed in Tables 2 or 3 of U.S. 20250188085.

In another embodiment, the RAS inhibitor is disclosed in U.S. 20250101043. In another embodiment, the RAS inhibitor is a compound of the formula disclosed in claim 1 of U.S. 20250101043. In another embodiment, the RAS inhibitor is a species disclosed in Tables 1, 2, or 7-11 of U.S20250101043.

In another embodiment, methods the disclosure further comprise administering one or more anticancer agents in combination, i.e., co-administered, with ESK981, or a pharmaceutically acceptable salt and/or solvate thereof. In another embodiment, the anticancer agent is a RAS inhibitor. In another embodiment, the anticancer agent is one or more of a chemotherapeutic agent, an immune checkpoint inhibitor, e.g., pembrolizumab, nivolumab, cemipilimab, atezolizumab, avelumab, durvalumab, ipilimumab, or radiation therapy.

A number of suitable anticancer agents are contemplated for use in the methods of the present disclosure. Indeed, the present disclosure contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-κB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present disclosure provide ESK981, or a pharmaceutically acceptable salt and/or solvate thereof, and at least one anti-hyperproliferative or antineoplastic agent selected from alkylating agents, antimetabolites, and natural products, e.g., herbs and other plant and/or animal derived compounds.

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide); and 17) gonadotropin-releasing hormone analogs (e.g., leuprolide).

Any oncolytic agent that is routinely used in a cancer therapy context finds use in the compositions and methods of the present disclosure. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1,serine-125 human interleukin-2) | | Emeryville, CA |
| Alemtuzumab | Campath | Millennium and ILEX |
| (IgG1κ anti CD52 antibody) | | Partners, LP, |
| | | Cambridge, MA |
| Alitretinoin | Panretin | Ligand |
| (9-cis-retinoic acid) | | Pharmaceuticals, Inc., |
| | | San Diego CA |
| Allopurinol (1,5-dihydro-4 H-pyrazolo[3,4- | Zyloprim | GlaxoSmithKline, |
| d]pyrimidin-4-one monosodium salt) | | Research Triangle Park, NC |
| Altretamine | Hexalen | US Bioscience, West |
| (N,N,N′,N′,N″,N″,- hexamethyl-1,3,5-triazine- | | Conshohocken, PA |
| 2,4,6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′- | | Pharmaceuticals, LP, |
| tetramethyl-5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck & Co., Inc., |
| (L-asparagine amidohydrolase, type EC-2) | | Whitehouse Station, NJ |
| BCG Live (lyophilized | TICE BCG | Organon Teknika, |
| preparation of an attenuated strain | | Corp., Durham, NC |
| of Mycobacterium bovis (Bacillus Calmette- | | |
| Gukin [BCG], substrain Montreal) | | |
| bexarotene capsules | Targretin | Ligand Pharmaceuticals |
| (4-[1-(5,6,7,8-tetrahydro-3,5,5,8,8- | | |
| pentamethyl-2-napthalenyl)ethenyl]benzoic | | |
| acid) | | |
| bexarotene gel | Targretin | Ligand Pharmaceuticals |
| Bleomycin (cytotoxic glycopeptide | Blenoxane | Bristol-Myers Squibb |
| antibiotics produced by Streptomyces | | Co., NY, NY |
| verticillus; bleomycin A₂ and | | |
| bleomycin B2) | | |
| Capecitabine (5′-deoxy-5-fluoro- | Xeloda | Roche |
| N-[(pentyloxy)carbonyl]-cytidine) | | |
| Carboplatin | Paraplatin | Bristol-Myers Squibb |
| (platinum, diammine [1,1- | | |
| cyclobutanedicarboxylato(2-)-0,0′]-,(SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford Pharmaceuticals, |
| | | Inc., Baltimore, MD |
| Celecoxib (as 4-[5-(4-methylphenyl)- | Celebrex | Searle Pharmaceuticals, |
| 3-(trifluoromethyl)-1H-pyrazol-1-yl] | | England |
| benzenesulfonamide) | | |
| Chlorambucil (4- | Leukeran | GlaxoSmithKline |
| [bis(2chlorethyl)amino]benzenebutanoic acid) | | |
| Cisplatin (PtCl₂H₆N₂) | Platinol | Bristol-Myers Squibb |
| Cladribine | Leustatin, 2- | R.W. Johnson |
| (2-chloro-2′-deoxy-b-D-adenosine) | CdA | Pharmaceutical |
| | | Research Institute, |
| | | Raritan, NJ |
| Cyclophosphamide | Cytoxan, | Bristol-Myers Squibb |
| (2-[bis(2-chloroethyl)amino]tetrahydro-2H- | Neosar | |
| 13,2-oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, |
| | | Inc., San Diego, CA |
| Dacarbazine | DTIC-Dome | Bayer AG, Leverkusen, |
| (5-(3,3-dimethyl-1-triazeno)-imidazole-4- | | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand |
| (recombinant peptide) | | Oaks, CA |
| daunorubicin liposomal | DanuoXome | Nexstar |
| ((8S-cis)-8-acetyl-10-[(3-amino-2,3,6- | | Pharmaceuticals, Inc., |
| trideoxy-á-L-lyxo-hexopyranosyl)oxy]- | | Boulder, CO |
| 7,8,9,10-tetrahydro-6,8,11-trihydroxy-1- | | |
| methoxy-5,12-naphthacenedione | | |
| hydrochloride) | | |
| Daunorubicin HCl, daunomycin | Cerubidine | Wyeth Ayerst, |
| ((1 S,3 S)-3-Acetyl-1,2,3,4,6,11-hexahydro- | | Madison, NJ |
| 3,5,12-trihydroxy-10-methoxy-6,11-dioxo-1- | | |
| naphthacenyl 3-amino-2,3,6-trideoxy-(alpha)- | | |
| L- lyxo -hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9- | | |
| one 4-acetate 2-benzoate, trihydrate) | | |
| Doxorubicin HCl | Adriamycin, | Pharmacia & Upjohn |
| (8S,10S)-10-[(3-amino-2,3,6-trideoxy-a-L- | Rubex | Company |
| lyxo-hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin | Pharmacia & Upjohn |
| | PFS Intravenous | Company |
| | injection | |
| doxorubicin liposomal | Doxil | Sequus |
| | | Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-,3- | | Company |
| [bis(2-chloroethyl)carbamate]17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, NJ |
| agent vidarabine, 9-b-D- | | |
| arabinofuranosyladenine (ara-A)) | | |
| Fluorouracil, 5-FU | Adrucil | ICN Pharmaceuticals, |
| (5-fluoro-2,4(1H,3H)-pyrimidinedione) | | Inc., Humacao, Puerto |
| | | Rico |
| Fulvestrant | Faslodex | IPR Pharmaceuticals, |
| (7-alpha-[9-(4,4,5,5,5-penta | | Guayama, Puerto Rico |
| fluoropentylsulphinyl)nonyl]estra-1,3,5-(10)- | | |
| triene-3,17-beta-diol) | | |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex | AstraZeneca |
| | Implant | Pharmaceuticals |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal | | |
| antibody Ibritumomab and the linker-chelator | | |
| tiuxetan [N-[2-bis(carboxymethyl)amino]-3- | | |
| (p-isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5,12-Naphthacenedione,9-acetyl-7-[(3- | | Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride,(7S- cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′,4′: | | |
| 6,7] indolizino[1,2-b] quinoline-3,14(4H,12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((—)—(S)-2,3,5, 6-tetrahydro-6-phenylimidazo | | Foundation, Titusville, |
| [2,1-b] thiazole monohydrochloride | | NJ |
| C₁₁H₁₂N₂S•HCl) | | |
| Lomustine | CeeNU | Bristol-Myers Squibb |
| (1-(2-chloro-ethyl)-3-cyclohexyl-1- | | |
| nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmith Kline |
| (4-[bis(2-chloroethyl)amino]-L- | | |
| phenylalanine) | | |
| Mercaptopurine, 6-MP | Purinethol | GlaxoSmithKline |
| (1,7-dihydro-6 H-purine-6-thione | | |
| monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7-one) | | Exton, Pa |
| Mitomycin C | Mutamycin | Bristol-Myers Squibb |
| mitomycin C | Mitozytrex | SuperGen, Inc., Dublin, |
| | | CA |
| Mitotane | Lysodren | Bristol-Myers Squibb |
| (1,1-dichloro-2-(o-chlorophenyl)-2-(p- | | |
| chlorophenyl)ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, Germany |
| Oprelvekin | Neumega | Genetics Institute, Inc., |
| (IL-11) | | Alexandria, VA |
| Oxaliplatin | Eloxatin | Sanofi Synthelabo, Inc., |
| (cis-[(1R,2R)-1,2-cyclohexanediamine-N,N′] | | NY, NY |
| [oxalato(2-)-O,O′]platinum) | | |
| Paclitaxel | TAXOL | Bristol-Myers Squibb |
| (5β, 20-Epoxy-1,2a, 4,7β, 10β, 13a- | | |
| hexahydroxytax-11-en-9-one 4,10-diacetate 2- | | |
| benzoate 13-ester with (2R,3S)-N-benzoyl-3- | | |
| phenylisoserine) | | |
| Pamidronate | Aredia | Novartis |
| (phosphonic acid (3-amino-1- | | |
| hydroxypropylidene)bis-, disodium salt, | | |
| pentahydrate, (APD)) | | |
| Pegademase | Adagen | Enzon Pharmaceuticals, |
| ((monomethoxypolyethylene glycol | (Pegademase | Inc., Bridgewater, NJ |
| succinimidyl)11-17-adenosine deaminase) | Bovine) | |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, |
| | | Inc., Vancouver, Canada |
| Procarbazine | Matulane | Sigma Tau |
| (N-isopropyl-μ-(2-methylhydrazino)-p- | | Pharmaceuticals, Inc., |
| toluamide monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, Inc., |
| (recombinant peptide) | | |
| Rituximab | Rituxan | Genentech, Inc., South |
| (recombinant anti-CD20 antibody) | | San Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]- | | |
| a(and b)-D-glucopyranose and 220 mg | | |
| citric acid anhydrous) | | |
| Talc (Mg₃Si₄O₁₀ (OH)₂) | Sclerosol | Bryan, Corp., Woburn, MA |
| Tamoxifen | Nolvadex | AstraZeneca |
| ((Z)2-[4-(1,2-diphenyl-1-butenyl)phenoxy]-N, | | Pharmaceuticals |
| N-dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]- | | |
| as-tetrazine-8-carboxamide) | | |
| teniposide, VM-26 | Vumon | Bristol-Myers Squibb |
| (4′-demethylepipodophyllotoxin 9-[4,6-0-(R)- | | |
| 2-thenylidene-(beta)-D-glucopyranoside]) | | |
| Testolactone | Teslac | Bristol-Myers Squibb |
| (13-hydroxy-3-oxo-13,17-secoandrosta-1,4- | | |
| dien-17-oic acid [dgr ]-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex Corporation |
| (Aziridine, 1,1′,1″-phosphinothioylidynetris-, | | |
| or Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′,4′:6,7] indolizino | | |
| [1,2-b] quinoline-3,14-(4H,12H)-dione | | |
| monohydrochloride) | | |
| Toremifene | Fareston | Roberts Pharmaceutical |
| (2-(p-[(Z)-4-chloro-1,2-diphenyl-1-butenyl]- | | Corp., Eatontown, NJ |
| phenoxy)-N,N-dimethylethylamine citrate | | |
| (1:1)) | | |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody | | |
| (I 131 is a radioimmunotherapeutic antibody)) | | |
| Trastuzumab | Herceptin | Genentech, Inc |
| (recombinant monoclonal IgG₁ kappa anti- | | |
| HER2 antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14- | Valstar | Anthra --> Medeva |
| valerate | | |
| ((2S-cis)-2-[1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds that have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafamib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, 5-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al, 2002.

The present disclosure provides methods for administering the ESK981, or a pharmaceutically acceptable salt and/or solvate thereof, with radiation therapy. The disclosure is not limited by the types, amounts, or delivery and administration systems used to deliver the therapeutic dose of radiation to the subject. For example, the subject may receive photon radiotherapy, particle beam radiation therapy, other types of radiotherapies, and combinations thereof. In some embodiments, the radiation is delivered to the subject using a linear accelerator. In still other embodiments, the radiation is delivered using a gamma knife.

The source of radiation can be external or internal to the subject. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by subjects. Internal radiation therapy involves implanting a radiation-emitting source, such as beads, wires, pellets, capsules, particles, and the like, inside the body at or near the tumor site including the use of delivery systems that specifically target cancer cells, e.g., using particles attached to cancer cell binding ligands. Such implants can be removed following treatment, or left in the body inactive. Types of internal radiation therapy include, but are not limited to, brachytherapy, interstitial irradiation, intracavity irradiation, radioimmunotherapy, and the like.

The subject may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a subject, so long as the dose of radiation is tolerated by the subject without unacceptable negative side-effects. Suitable types of radiotherapy include, for example, ionizing (electromagnetic) radiotherapy, e.g., X-rays or gamma rays, or particle beam radiation therapy, e.g., high linear energy radiation. Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to a subject is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present disclosure.

In some embodiments of the present disclosure, ESK981, or a pharmaceutically acceptable salt and/or solvate thereof, and one or more anticancer agents, e.g., a RAS inhibitor, are administered to a subject under one or more of the following conditions: at different periodicities, at different durations, at different concentrations, and/or by different administration routes. In some embodiments, ESK981, or a pharmaceutically acceptable salt and/or solvate thereof, is administered prior to the anticancer agent, e.g., 0.5, 1, 2, 3, 4, 5, 10, 12, or 18 hours, 1, 2, 3, 4, 5, or 6 days, or 1, 2, 3, or 4 weeks prior to the administration of anticancer agent. In some embodiments, ESK981 is administered after the anticancer agent, e.g., 0.5, 1, 2, 3, 4, 5, 10, 12, or 18 hours, 1, 2, 3, 4, 5, or 6 days, or 1, 2, 3, or 4 weeks after the administration of the anticancer agent. In some embodiments, ESK981, or a pharmaceutically acceptable salt and/or solvate thereof, and the anticancer agent are administered concurrently but on different schedules, e.g., the compound is administered daily while the therapeutic or anticancer agent is administered once a week, once every two weeks, once every three weeks, or once every four weeks. In other embodiments, ESK981, or a pharmaceutically acceptable salt and/or solvate thereof, is administered once a week while the anticancer agent is administered daily, once a week, once every two weeks, once every three weeks, or once every four weeks.

Compositions within the scope of this disclosure include all compositions wherein ESK981 is contained in an amount that is effective to achieve its intended purpose. While individual needs vary, determination of optimal ranges of effective amounts of each component is within the skill of the art. Typically, ESK981, or a pharmaceutically acceptable salt and/or solvate thereof, may be administered to subjects, e.g., human cancer patients, orally at a dose of 0.0025 to 100 mg/kg, or an equivalent amount of the pharmaceutically acceptable salt thereof, per day of the body weight of the mammal being treated for disorders responsive to induction of apoptosis.

In one embodiment, about 0.01 to about 25 mg/kg of ESK981, or a pharmaceutically acceptable salt and/or solvate thereof, is orally administered to treat, ameliorate, or prevent prostate cancer or pancreatic cancer, e.g., NEPC, PDAC, PNETs, or NECs. For intramuscular injection, the dose is generally about one-half of the oral dose. For example, a suitable intramuscular dose would be about 0.0025 to about 25 mg/kg, or from about 0.01 to about 5 mg/kg.

In some embodiments, from about 25 mg to about 1000 mg of ESK981, or a pharmaceutically acceptable salt and/or solvate thereof, is orally administered to the subject per day in combination with a RAS inhibitor. In some embodiments, from about 50 mg to about 500 mg of ESK981 is orally administered to the subject per day in combination with a RAS inhibitor. In some embodiments about 50 mg, about 100 mg, about 120 mg, about 140 mg, about 160 mg, about 180 mg, about 200 mg, about 220 mg, about 240 mg, about 260 mg, about 280 mg, or about 300 mg of ESK981 is orally administered per day in combination with a RAS inhibitor.

The unit oral dose may comprise from about 0.01 to about 1000 mg, for example, about 0.1 to about 100 mg of ESK981, or a pharmaceutically acceptable salt and/or solvate thereof. The unit dose may be administered one or more times daily as one or more tablets or capsules each containing from about 0.1 to about 10 mg, about 0.25 to 50 mg of ESK981, or a pharmaceutically acceptable salt and/or solvate thereof.

In a topical formulation, ESK981 may be present at a concentration of about 0.01 to 100 mg per gram of carrier. In a one embodiment, ESK981 is present at a concentration of about 0.07-1.0 mg/ml, for example, about 0.1-0.5 mg/ml, and in one embodiment, about 0.4 mg/ml.

In addition to administering ESK981, or a pharmaceutically acceptable salt and/or solvate thereof, as a raw chemical, the ESK981, or a pharmaceutically acceptable salt and/or solvate thereof, may be administered as part of a pharmaceutical formulation containing suitable pharmaceutically acceptable carriers comprising excipients and auxiliaries that facilitate processing of ESK981, or a pharmaceutically acceptable salt and/or solvate thereof, into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of ESK981, or a pharmaceutically acceptable salt and/or solvate thereof, together with the excipient.

The pharmaceutical compositions comprising ESK981, or a pharmaceutically acceptable salt and/or solvate thereof, may be administered to any subject that may experience the beneficial effects of ESK981. Foremost among such subjects are mammals, e.g., humans, although the disclosure is not intended to be so limited. Other subjects include veterinary animals (cows, sheep, pigs, horses, dogs, cats and the like).

ESK981, or a pharmaceutically acceptable salt and/or solvate thereof, and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present disclosure are manufactured in a manner which is itself known, for example, by means of conventional mixing, granulating, dragee-making, dissolving, or lyophilizing processes. Thus, pharmaceutical preparations for oral use can be obtained by combining ESK981, or a pharmaceutically acceptable salt and/or solvate thereof, with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this disclosure are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than Cu). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one that includes about 30% almond oil and about 70% white soft paraffin by weight. Lotions may be conveniently prepared by dissolving the active ingredient, in a suitable high molecular weight alcohol such as propylene glycol or polyethylene glycol.

In another embodiment, present disclosure provides methods of treating a subject having cancer, e.g., prostate cancer, colorectal cancer, non-small cell lung cancer, and pancreatic cancer, e.g., PDAC, PNETs, or NECs, comprising (a) determining whether a biomarker is present or absent in a biological sample taken from the subject; and (b) administering a therapeutically effective amount of ESK981, or a pharmaceutically acceptable salt and/or solvate thereof and, optionally, one or more additional anticancer agents, e.g., a RAS inhibitor, to the subject if the biomarker is present in the biological sample. See, e.g., Goossens et al., Transl Cancer Res. 4:256-269 (2015); Kamel and Al-Amodi, Genomics Proteomics Bioinformatics 15:220-235 (2017); and Konikova and Kusenda, Neoplasma 50:31-40 (2003).

Biomarkers include, but are not limited to, KRAS mutations and/or Trp53 mutations.

In one embodiment, the biomarker is a KRAS mutation, which is differentially present in a subject of one phenotypic status, e.g., a subject having PDAC, as compared with another phenotypic status, e.g., a normal undiseased subject or a subject having cancer without PDAC. In one embodiment, the biomarker is Kras^{G12D}. In another embodiment, the biomarker is Kras^{G12C}. In another embodiment, the biomarker is Kras^{G12V}. In another embodiment, the biomarker is Kras^{G13C}. In another embodiment, the biomarker is Kras^{Q61H}.

In one embodiment, the biomarker is a Trp53 mutation, which is differentially present in a subject of one phenotypic status, e.g., a subject having PDAC, as compared with another phenotypic status, e.g., a normal undiseased subject or a subject having cancer without PDAC. In one embodiment, the biomarker is Trp53^{R172H}.

Biomarker standards can be predetermined, determined concurrently, or determined after a biological sample is obtained from the subject. Biomarker standards for use with the methods described herein can, for example, include data from samples from subjects without cancer; data from samples from subjects with cancer, e.g., prostate cancer, that is not metastatic; and data from samples from subjects with cancer, e.g., prostate cancer and pancreatic cancer, that is metastatic. Comparisons can be made to establish predetermined threshold biomarker standards for different classes of subjects, e.g., diseased vs. undiseased subjects. The standards can be run in the same assay or can be known standards from a previous assay.

A biomarker is differentially present between different phenotypic status groups if the mean or median expression or mutation levels of the biomarker is calculated to be different, i.e., higher or lower, between the groups. Thus, biomarkers provide an indication that a subject, e.g., a cancer patient, belongs to one phenotypic status or another.

The determination of the expression level or mutation status of a biomarker in a subject can be performed using any of the many methods known in the art. Any method known in the art for quantitating specific proteins and/or detecting a KRAS mutation, a Trp53 mutation, or the expression or mutation levels of any other biomarker in a subject or a biological sample may be used in the methods of the disclosure. Examples include, but are not limited to, PCR (polymerase chain reaction), or RT-PCR, flow cytometry, Northern blot, Western blot, ELISA (enzyme linked immunosorbent assay), RIA (radioimmunoassay), gene chip analysis of RNA expression, immunohistochemistry or immunofluorescence. See, e.g., Slagle et al., Cancer 83:1401 (1998); Hudlebusch et al., Clin Cancer Res 17:2919-2933 (2011). Certain embodiments of the disclosure include methods wherein biomarker RNA expression (transcription) is determined. Other embodiments of the disclosure include methods wherein protein expression in the biological sample is determined. See, e.g., Harlow et al., Antibodies: A Laboratory Manual, Cold Spring Harbor Laboratory, Cold Spring Harbor, NY, (1988); Ausubel et al., Current Protocols in Molecular Biology, John Wiley & Sons, New York 3rd Edition, (1995); Kamel and Al-Amodi, Genomics Proteomics Bioinformatics 15:220-235 (2017). For northern blot or RT-PCR analysis, RNA is isolated from the tumor tissue sample using RNAse free techniques. Such techniques are commonly known in the art.

In one embodiment of the disclosure, a biological sample is obtained from the subject and the biological sample is assayed for determination of a biomarker expression or mutation status.

In another embodiment, the present disclosure provides a method of treating a subject having NEPC, PDAC, PNETs, NECs, CRC, or NSCLC, the method comprising:
(a) determining whether a KRAS mutation and/or a Trp53 mutation is present or absent in a biological sample taken from the subject; and
(b) administering a therapeutically effective amount of ESK981, or a pharmaceutically acceptable salt and/or solvate thereof, and optionally a RAS inhibitor to the subject if a KRAS mutation and/or a Trp53 mutation is present in the biological sample.

In another embodiment, the present disclosure provides a method, comprising administering a therapeutically effective amount of ESK981, or a pharmaceutically acceptable salt and/or solvate thereof, and optionally a RAS inhibitor to a subject in need thereof, wherein:
(a) the subject has NEPC, PDAC, PNETs, NECs, CRC, or NSCLC; and
(b) the NEPC, PDAC, PNETs, NECs, CRC, or NSCLC is characterized as having a KRAS mutation and/or a Trp53 mutation.

In another embodiment, the present disclosure provides a method of identifying whether a subject having NEPC, PDAC, PNETs, NECs, CRC, or NSCLC is a candidate for treatment with ESK981, or a pharmaceutically acceptable salt and/or solvate thereof, and optionally a RAS inhibitor, the method comprising:
(a) determining whether a KRAS mutation and/or a Trp53 mutation is present or absent in a biological sample taken from the subject; and
(b) identifying the subject as being a candidate for treatment if the KRAS mutation and/or a Trp53 mutation is present; or
(c) identifying the subject as not being a candidate for treatment if the KRAS mutation and/or a Trp53 mutation is absent.

In another embodiment, the present disclosure provides a method of predicting treatment outcome in a subject having NEPC, PDAC, PNETs, NECs, CRC, or NSCLC, the method comprising determining whether a KRAS mutation and/or a Trp53 mutation is present or absent in a biological sample taken from the subject, wherein:
(a) the presence of a KRAS mutation and/or a Trp53 mutation in the biological sample indicates that administering ESK981, or a pharmaceutically acceptable salt and/or solvate thereof, and optionally a RAS inhibitor to the subject will likely cause a favorable therapeutic response; and
(b) the absence of a KRAS mutation and/or a Trp53 mutation in the biological sample indicates that administering ESK981, or a pharmaceutically acceptable salt and/or solvate thereof, and optionally a RAS inhibitor to the subject will likely cause an unfavorable therapeutic response.

In another embodiment, the KRAS mutation is Kras^{G12D}.

In another embodiment, the Trp53 mutation is Trp53^{R172H}.

In another embodiment, the KRAS mutation is Kras^{G12C}.

In another embodiment, the KRAS mutation is Kras^{G12V}.

In another embodiment, the KRAS mutation is Kras^{G13C}.

In another embodiment, the KRAS mutation is Kras^{Q61H}.

The term “ESK981” refers to 13-isobutyl-4-methyl-10-(pyrimidin-2-ylamino)-1,2,4,7,8,13-hexahydro-6H-indazolo[5,4-a]pyrrolo[3,4-c]carbazol-6-one:

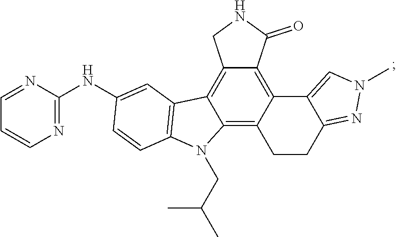 [SMILES: CC(C)Cn1c2ccc(Nc3ncccn3)cc2c2c3c(c4c(c21)CCc1nn(C)cc1-4)C(=O)NC3]

ESK981 (formerly known as CEP-11981) is an oral multi-tyrosine kinase inhibitor (MTKI). Hudkins et al., J. Med. Chem. 55:903-913 (2012); Invest New Drugs. 2014; 32(6):1258-68. ESK981 also inhibits autophagy through direct targeting of the lipid kinase PIKfyve. Qiao et al., Nat Cancer. 2021; 2:978-93; WO 2022/094058.

The term “ESK981 Solid Form” as used herein refers to any amorphous or crystalline form of ESK981, and pharmaceutically acceptable salts and/or solvates thereof. Crystalline forms of ESK981 may exhibit polymorphism. Polymorphic forms of ESK981, and pharmaceutically acceptable salt and/or solvates thereof, referred to herein as collectively as “ESK981 Polymorphs” or individually as an “ESK981 Polymorph,” include but are not limited to, ESK981 pTSA-A₁, ESK981 pTSA-A₂, ESK981 pTSA-B₂, and ESK981 pTSA-C₂, as described below and in U.S. Pat. No. 8,513,272; the polymorphic forms referred to as the “A₀,” “B₀,” “C₀,” “D₀,” “S₁,” “S₂,” “S₃” “S₄,” “S₆,” “S₇,” “S₈,” “S₉,” “S₁₀,” “S₁₃,” S₁₄,” “S₁₅,” “S₁₆,” “S₁₇,” “S₁₈,” “S₁₉,” and “S₂₀” forms in U.S. Pat. No. 8,513,272; and ESK981 AA as described below and in U.S. Pat. No. 8,884,014. U.S. Pat. Nos. 8,513,272 and 8,884,014 are incorporated by reference herein in their entirety.

The term “ESK981 pTSA-A₁” refers to a crystalline para-toluenesulfonic acid (pTSA) salt of ESK981 having an ESK981:pTSA: ratio of 1:1. The preparation and characterization of ESK981 pTSA-A₁ is described in U.S. Pat. No. 8,513,272. ESK981 pTSA-A₁ is characterized as having an x-ray powder diffraction (XRPD) pattern comprising any three or more peaks listed in Table A using Cu Kα radiation, wherein the 2Θ values listed in Table A are ±0.2 degrees 2Θ. In one embodiment, ESK981 pTSA-A₁ is characterized as having an XRPD pattern comprising peaks at 5.37, 6.79, 13.64, 22.58, and 25.54 degrees 2Θ using Cu Kα radiation, wherein the 2Θ values are ±0.2 degrees 2Θ. The principle onset temperature of ESK981 pTSA-A₁ as determined by differential scanning calorimetry (DSC) is 262.8° C. and the peak temperature is 266.4° C.

TABLE A
ESK981 pTSA-A₁ XRPD peaks

| Peak No. | Angle (°2 Theta) | d-spacing (Å) | Intensity (%) |
|---|---|---|---|
| 1 | 5.37 | 16.45 | 100 |
| 2 | 6.79 | 13.02 | 70 |
| 3 | 11.59 | 7.63 | 8 |
| 4 | 12.65 | 6.99 | 11 |
| 5 | 12.70 | 6.98 | 10 |
| 6 | 13.64 | 6.48 | 20 |
| 7 | 15.94 | 5.56 | 6 |
| 8 | 17.37 | 5.10 | 6 |
| 9 | 19.26 | 4.60 | 7 |
| 10 | 20.34 | 4.36 | 6 |
| 11 | 21.03 | 4.22 | 9 |
| 12 | 22.58 | 3.93 | 13 |
| 13 | 25.54 | 3.48 | 25 |
| 14 | 25.91 | 3.44 | 8 |

The term “ESK981 pTSA-A₂” refers to a crystalline pTSA salt of ESK981 having an ESK981:pTSA ratio of 1:2. The preparation and characterization of ESK981 pTSA-A₂ is described in U.S. Pat. No. 8,513,272. ESK981 pTSA-A₂ is characterized as having an XRPD pattern with any three or more peaks listed in Table B using Cu Kα radiation, wherein the 2Θ values listed in Table B are ±0.2 degrees 2Θ. In one embodiment, ESK981 pTSA-A₂ is characterized as having an XRPD pattern comprising peaks at 5.63, 8.48, 12.46, 18.21, and 23.95 degrees 2Θ using Cu Kα radiation, wherein the 2Θ values are ±0.2 degrees 2Θ. The principle onset temperature of ESK981 pTSA-A₂ as determined by DSC is 273.5° C. and the peak temperature is 278.4° C.

TABLE B
ESK981 pTSA-A₂ XRPD peaks

| Peak No. | Angle (°2 Theta) | d-spacing (Å) | Intensity (%) |
|---|---|---|---|
| 1 | 3.71 | 23.80 | 26 |
| 2 | 5.63 | 15.69 | 100 |
| 3 | 8.48 | 10.43 | 90 |
| 4 | 11.30 | 7.83 | 26 |
| 5 | 12.46 | 7.11 | 34 |
| 6 | 13.26 | 6.68 | 13 |
| 7 | 14.05 | 6.30 | 5 |
| 8 | 14.49 | 6.11 | 11 |
| 9 | 14.94 | 5.93 | 12 |
| 10 | 15.52 | 5.71 | 13 |
| 11 | 16.99 | 5.22 | 25 |
| 12 | 17.02 | 5.21 | 24 |
| 13 | 17.46 | 5.08 | 6 |
| 14 | 18.21 | 4.87 | 31 |
| 15 | 20.28 | 4.38 | 20 |
| 16 | 20.56 | 4.32 | 7 |
| 17 | 21.97 | 4.05 | 13 |
| 18 | 22.52 | 3.95 | 11 |
| 19 | 22.91 | 3.88 | 10 |
| 20 | 23.95 | 3.72 | 29 |
| 21 | 25.18 | 3.54 | 23 |
| 22 | 25.55 | 3.49 | 12 |
| 23 | 26.60 | 3.35 | 7 |

The term “ESK981 pTSA-B₂” refers to a crystalline pTSA salt of ESK981 having an ESK981:pTSA ratio of 1:2. The preparation and characterization of ESK981 pTSA-B₂ is described in U.S. Pat. No. 8,513,272. ESK981 pTSA-B₂ is characterized as having an XRPD pattern with any three or more peaks listed in Table C using Cu Kα radiation, wherein the 2Θ values listed in Table C are ±0.2 degrees 2Θ. In one embodiment, ESK981 pTSA-B₂ is characterized as having an XRPD pattern comprising peaks at 3.75, 5.63, 8.40, 11.31, and 15.12 degrees 2Θ using Cu Kα radiation, wherein the 2Θ values are ±0.2 degrees 2Θ. The principle onset temperature of ESK981 pTSA-B₂ as determined by DSC is 277.2° C. and the peak temperature is 280.2° C.

TABLE C
ESK981 pTSA-B₂ XRPD peaks

| Peak No. | Angle (°2 Theta) | d-spacing (Å) | Intensity (%) |
|---|---|---|---|
| 1 | 3.75 | 23.54 | 39 |
| 2 | 5.63 | 15.69 | 100 |
| 3 | 7.53 | 11.74 | 6 |
| 4 | 8.40 | 10.52 | 87 |
| 5 | 11.31 | 7.83 | 36 |
| 6 | 12.11 | 7.31 | 13 |
| 7 | 12.46 | 7.10 | 8 |
| 8 | 12.75 | 6.94 | 7 |
| 9 | 13.17 | 6.72 | 18 |
| 10 | 14.12 | 6.27 | 8 |
| 11 | 14.73 | 6.01 | 25 |
| 12 | 15.12 | 5.86 | 32 |
| 13 | 16.42 | 5.40 | 6 |
| 14 | 16.87 | 5.26 | 22 |
| 15 | 17.00 | 5.22 | 21 |
| 16 | 17.29 | 5.13 | 8 |
| 17 | 17.87 | 4.97 | 8 |
| 18 | 18.14 | 4.89 | 6 |
| 19 | 18.61 | 4.77 | 7 |
| 20 | 20.15 | 4.41 | 20 |
| 21 | 22.52 | 3.95 | 5 |
| 22 | 24.03 | 3.70 | 20 |
| 23 | 24.60 | 3.62 | 21 |
| 24 | 24.78 | 3.59 | 11 |
| 25 | 25.47 | 3.50 | 12 |
| 26 | 26.12 | 3.41 | 5 |

The term “ESK981 pTSA-C₂” refers to a crystalline pTSA salt of ESK981 having an ESK981:pTSA ratio of 1:2. The preparation and characterization of ESK981 pTSA-C₂ is described in U.S. Pat. No. 8,513,272. ESK981 pTSA-C₂ is characterized as having an XRPD pattern with any three or more peaks listed in Table D using Cu Kα radiation, wherein the 2Θ values listed in Table D are ±0.2 degrees 2Θ. In one embodiment, ESK981 pTSA-C₂ is characterized as having an XRPD pattern comprising peaks at 5.72, 8.49, 11.37, 13.26, and 16.95 degrees 2Θ using Cu Kα radiation, wherein the 2Θ values are ±0.2 degrees 2Θ. The principle onset temperature of ESK981 pTSA-C₂ as determined by DSC is 277.5° C. and the peak temperature is 280.4° C.

TABLE D
ESK981 pTSA-C₂ XRPD peaks

| Peak No. | Angle (°2 Theta) | d-spacing (Å) | Intensity (%) |
|---|---|---|---|
| 1 | 3.84 | 23.00 | 6 |
| 2 | 5.72 | 15.45 | 50 |
| 3 | 8.49 | 10.41 | 100 |
| 4 | 11.37 | 7.77 | 35 |
| 5 | 12.13 | 7.29 | 5 |
| 6 | 13.26 | 6.67 | 29 |
| 7 | 14.19 | 6.24 | 8 |
| 8 | 14.81 | 5.98 | 7 |
| 9 | 15.17 | 5.84 | 14 |
| 10 | 16.95 | 5.23 | 40 |
| 11 | 17.06 | 5.19 | 22 |
| 12 | 17.37 | 5.10 | 16 |
| 13 | 21.55 | 4.12 | 7 |
| 14 | 24.11 | 3.69 | 6 |

The term “ESK981 AA” refers to a crystalline acetic acid (CH₃CO₂H) salt of ESK981 having an ESK981:CH₃CO₂H ratio of 1:2. The preparation and characterization of ESK981 AA is described in U.S. Pat. No. 8,884,014. ESK981 AA is characterized as having an XRPD pattern using Cu Kα radiation with any three or more peaks listed in Table E, wherein the 2Θ values listed in Table E are ±0.2 degrees 2Θ. In one embodiment, ESK981 AA is characterized as having an XRPD pattern comprising peaks at 6.17, 6.44, 14.36, 20.69, and 26.09 degrees 2Θ using Cu Kα radiation, wherein the 2Θ values are ±0.2 degrees 2Θ.

TABLE E
ESK981 AA XRPD peaks

| Peak No. | Angle (°2 Theta) | d-spacing (Å) | Intensity (%) |
|---|---|---|---|
| 1 | 5.19 | 17.01 | 17.5 |
| 2 | 6.17 | 14.31 | 53.3 |
| 3 | 6.44 | 13.71 | 100.0 |
| 4 | 8.38 | 10.54 | 1.7 |
| 5 | 10.51 | 8.41 | 13.0 |
| 6 | 11.03 | 8.01 | 8.6 |
| 7 | 12.34 | 7.17 | 8.1 |
| 8 | 13.19 | 6.71 | 2.0 |
| 9 | 14.36 | 6.16 | 29.7 |
| 10 | 15.20 | 5.82 | 1.2 |
| 11 | 15.84 | 5.59 | 14.9 |
| 12 | 16.19 | 5.47 | 8.5 |
| 13 | 17.19 | 5.15 | 4.8 |
| 14 | 17.51 | 5.06 | 4.1 |
| 15 | 18.33 | 4.84 | 16.7 |
| 16 | 19.51 | 4.55 | 2.8 |
| 17 | 20.69 | 4.29 | 17.2 |
| 18 | 21.51 | 4.13 | 5.9 |
| 19 | 22.39 | 3.97 | 5.1 |
| 20 | 22.98 | 3.87 | 8.5 |
| 21 | 23.71 | 3.75 | 13.1 |
| 22 | 24.89 | 3.57 | 5.4 |
| 23 | 26.09 | 3.41 | 33.8 |
| 24 | 26.73 | 3.33 | 4.0 |
| 25 | 27.84 | 3.21 | 1.5 |
| 26 | 28.28 | 3.15 | 1.5 |
| 27 | 28.55 | 3.12 | 1.6 |
| 28 | 29.52 | 3.02 | 2.9 |
| 29 | 29.85 | 2.99 | 5.5 |
| 30 | 30.87 | 2.89 | 2.5 |
| 31 | 32.06 | 2.79 | 1.6 |
| 32 | 33.02 | 2.71 | 2.5 |
| 33 | 33.80 | 2.65 | 1.0 |
| 34 | 34.13 | 2.62 | 1.0 |
| 35 | 34.84 | 2.57 | 2.7 |
| 36 | 35.21 | 2.55 | 2.1 |
| 37 | 36.38 | 2.47 | 3.4 |
| 38 | 36.81 | 2.44 | 5.9 |

The term “apilimod” refers to N-[(E)-(3-methylphenyl)methylideneamino]-6-morpholin-4-yl-2-(2-pyridin-2-ylethoxy)pyrimidin-4-amine:

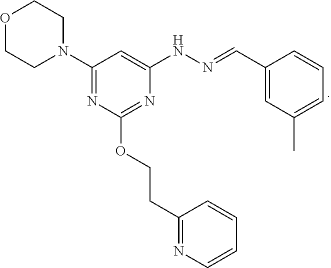 [SMILES: Cc1cccc(/C=N/Nc2cc(N3CCOCC3)nc(OCCc3ccccn3)n2)c1]

Apilimod (formerly known as STA-5326) is an interleukins IL-12 and IL-23 inhibitor that was initially developed for the treatment of autoimmune conditions like Crohn's disease and rheumatoid arthritis See e.g., Billich A. IDrugs 2007:10(1):53-59. Apilimod also inhibits autophagy by inhibiting lipid kinase enzyme PIKfyve See, e.g., Shisheva, A. et al., Molec. and Cell. Bio. 1999; 64(6):1750-1755; Cai, X. Chem. & Bio. 2013; 20(7):912-921.

The term “anticancer agent” as used herein, refer to any therapeutic agent, e.g., chemotherapeutic compounds and/or molecular therapeutic compounds, antisense therapies, radiation therapies, surgical interventions, or combinations thereof, used in the treatment of hyperproliferative diseases such as cancer, e.g., in mammals, e.g., in humans.

The term “RAS inhibitor” refers to a compound that inhibits RAS and/or RAS-mutant proteins. A RAS inhibitor can be, for example, a “pan-RAS inhibitor,” i.e., a compound that inhibits multiple RAS family members, e.g., KRAS, NRAS, and/or HRAS, or a KRAS-MAPK inhibitor.

The terms “KRAS-MAPK inhibitor” or “KRAS inhibitor” refer to a compound that inhibits KRAS and/or KRAS-mutant, e.g., G12C or G12D mutant, proteins. KRAS-MAPK inhibitors are known in the art and include, but are not limited to, ARS-853, ARS-1620, AMG510 (Sotorasib), MRTX849, (Adagrasib), MRTX-EX185, MRTX-1133, ASP2453, RMC-6291, RMC-6236, RMC-036, RMC-037, BBO-8520, ERAS-3490, and JDQ443. See Tria et al., Cancers 15(8): 2375 (2023); https://doi.org/10.3390/cancers15082375.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of prostate cancer or pancreatic cancer, e.g., PDAC, PNETs, or NECs, in one embodiment, a therapeutically effective amount will refer to the amount of a therapeutic agent that decreases the rate of tumor growth, decreases tumor mass, decreases the number of metastases, increases time to tumor progression, or increases survival time by at least 1%, at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The term “hyperproliferative disease,” as used herein, refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “neoplastic disease,” as used herein, refers to any abnormal growth of cells being either benign (non-cancerous) or malignant (cancerous).

The term “normal cell,” as used herein, refers to a cell that is not undergoing abnormal growth or division. Normal cells are non-cancerous and are not part of any hyperproliferative disease or disorder.

The term “KRAS mutation” and the like as used herein refers to a mutated Kirsten rat sarcoma viral oncogene homologue (KRAS) oncogene. KRAS mutations are dominated by single-base missense mutations, 98% of which are found at codon 12 (G12), codon 13 (G13), or codon 61 (Q61). See, e.g., Huang et al., Signal Transduction and Targeted Therapy 2021; 6:Article 386.

The term “Trp53 mutation” and the like as used herein refers to a mutated transformation related protein 53 gene. This gene encodes tumor protein p53. Trp53 mutations have been shown to synergize with loss-of-function mutations in other tumor suppressor genes generally accelerating tumor development and progression. See, e.g., Hingorani et al., Cancer Cell 2002; 7(5) 469-83.

The terms “a” and “an” refer to one or more.

The term “about,” as used herein, includes the recited number±10%. Thus, “about 10” means 9 to 11.

The terms “treat,” “treating,” “treatment,” and the like as used herein refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of ESK981 to a subject in need of such treatment.

Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

The terms “prevent,” “preventing,” and “prevention,” as used herein, refer to a decrease in the occurrence of pathological cells, e.g., hyperproliferative or neoplastic cells, in an subject. The prevention may be complete, e.g., the total absence of pathological cells in a subject. The prevention may also be partial, such that the occurrence of pathological cells in a subject is less than that which would have occurred without the present disclosure.

The term “biological sample” as used herein refers any tissue or fluid from a subject that is suitable for detecting a biomarker, e.g., KRAS mutation and/or a Trp53 mutation. Examples of useful biological samples include, but are not limited to, biopsied tissues and/or cells, e.g., solid tumor, lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for genetic aberrations using any technique known in the art. Such techniques include, but are not limited to, polymerase chain reaction (PCR) methodology, reverse transcription-polymerase chain reaction (RT-PCR) methodology, or cytoplasmic light chain immunofluorescence combined with fluorescence in situ hybridization (cIg-FISH). A biological sample can be obtained using techniques that are well within the scope of ordinary knowledge of a clinical practioner. In one embodiment of the disclosure, the biological sample comprises blood cells.

The phrase “in combination” as used in connection with the administration ESK981, or a pharmaceutically acceptable salt and/or solvate thereof, and one or more additional anticancer agents, e.g., pan-RAS inhibitors or KRAS-MAPK inhibitors, to a subject means that the ESK981, or a pharmaceutically acceptable salt and/or solvate thereof, and one or more additional anticancer agents can be administered to the subject together, e.g., as part of a single pharmaceutical composition or formulation, or separately, e.g., as part of two or more separate pharmaceutical compositions or formulations. The phrase “in combination” as used in connection with the administration of ESK981, or a pharmaceutically acceptable salt and/or solvate thereof, and one or more additional anticancer agents to a subject is thus intended to embrace administration of ESK981, or a pharmaceutically acceptable salt and/or solvate thereof, and one or more additional anticancer agents in a sequential manner, wherein ESK981, or a pharmaceutically acceptable salt and/or solvate thereof, and one or more additional anticancer agents are administered to the subject at a different time, as well as administration concurrently, or in a substantially simultaneous manner. Simultaneous administration can be accomplished, for example, by administering to the subject a single capsule having a fixed ratio of each of ESK981, or a pharmaceutically acceptable salt and/or solvate thereof, and the one or more additional anticancer agents or in multiple, single capsules, tablets, etc. for each of ESK981, or a pharmaceutically acceptable salt and/or solvate thereof, and the one or more additional anticancer agents. Sequential or substantially simultaneous administration of the ESK981, or a pharmaceutically acceptable salt and/or solvate thereof, and the one or more additional anticancer agents can be accomplished by any appropriate route including, but not limited to, oral routes, intravenous routes, intramuscular routes, and direct absorption through mucous membrane tissues. ESK981, or a pharmaceutically acceptable salt and/or solvate thereof, and the one or more additional anticancer agents can be administered by the same route or by different routes. For example, an anticancer agent of the combination may be administered by intravenous injection while ESK981, or a pharmaceutically acceptable salt and/or solvate thereof, of the combination may be administered orally. Alternatively, for example, both ESK981, or a pharmaceutically acceptable salt and/or solvate thereof and the one or more additional anticancer agents may be administered orally or both ESK981, or a pharmaceutically acceptable salt and/or solvate thereof and the one or more additional anticancer agents may be administered by intravenous injection. ESK981, or a pharmaceutically acceptable salt and/or solvate thereof, and one or more additional anticancer agents may also be administered in alternation. In one embodiment, ESK981 and the one or more additional anticancer agents are administered to a subject separately, e.g., as part of two or more separate pharmaceutical compositions or formulations. The same principles apply when ESK981, or a pharmaceutically acceptable salt and/or solvate thereof, and two or more anticancer agents are administered in combination to a subject. In some embodiments, ESK981, or a pharmaceutically acceptable salt and/or solvate thereof, is administered to a subject in combination with a RAS inhibitor. In some embodiments, ESK981, or a pharmaceutically acceptable salt and/or solvate thereof, is administered to a subject in combination with a pan-RAS inhibitor. In some embodiments, ESK981, or a pharmaceutically acceptable salt and/or solvate thereof, is administered to a subject in combination with a KRAS-MAPK inhibitor.

The term “one or more” as used herein means that at least one component in a relevant category of components, e.g. one or more anticancer agents, or at least one component in a disclosed list, e.g., one or more antineoplastic agents of Table 1, is present and, in some embodiments, more than one component is present. In some embodiments, one or more means 1 to 50. In some embodiments, one or more means 1 to 40. In some embodiments, one or more means 1 to 30. In some embodiments, one or more means 1 to 20. In some embodiments, one or more means 1 to 10. In some embodiments, one or more means 1 to 5. In some embodiments, one or more means 1 to 3. In some embodiments, one or more means 1 or 2. In some embodiments, one or more means 1.

It is understood that wherever aspects or embodiments of the present disclosure are described herein with the language “comprising,” otherwise analogous aspects or embodiments described in terms of “consisting of” and/or “consisting essentially of” are also provided.

### Kits

In another embodiment, the disclosure provides a kit comprising a PIKfyve inhibitor, e.g., ESK981, or pharmaceutically acceptable salt or solvate thereof, and a label with instructions for how to use the PIKfyve inhibitor to treat cancer in a subject

In another embodiment, the disclosure provides a kit for carrying out the methods of the present disclosure, the kit comprising a PIKfyve inhibitor, e.g., ESK981, or pharmaceutically acceptable salt or solvate thereof, and a label with instructions for how to use the PIKfyve inhibitor to treat cancer in a subject.

In another embodiment, the label is approved by at least one regulatory health agency, e.g., the United States Food and Drug Administration (FDA), the European Medicines Agency (EMA), the China Food and Drug Administration (CFDA), or the Japanese Ministry of Health Labor and Welfare (MHLW).

### Particular Embodiments

The disclosure provides the following particular embodiments.

Embodiment I. A method of treating cancer in a subject in need thereof, the method comprising administering to the subject a therapeutically effective amount of 13-isobutyl-4-methyl-10-(pyrimidin-2-ylamino)-1,2,4,7,8,13-hexahydro-6H-indazolo[5,4-a]pyrrolo[3,4-c]carbazol-6-one (ESK981), wherein the cancer is neuroendocrine prostate cancer (NEPC), pancreatic ductal adenocarcinoma (PDAC), pancreatic neuroendocrine tumors (PNETs), or pancreatic neuroendocrine carcinomas (NECs).

Embodiment II. The method of Embodiment I, wherein the cancer is NEPC.

Embodiment III. The method of Embodiment I, wherein the cancer is PDAC.

Embodiment IV. The method of Embodiment I, wherein the cancer is PNETs.

Embodiment V. The method of Embodiment I, wherein the cancer is NECs.

Embodiment VI. The method of any one of Embodiments I-V, comprising administering ESK981 to the subject in combination with a therapeutically effective amount of at least one anticancer agent to the subject.

Embodiment VII. The method of Embodiment VI, wherein the at least one anticancer agent comprises a chemotherapeutic agent, an immune checkpoint inhibitor, or radiation therapy.

Embodiment VIII. The method of Embodiments VI or VII, wherein the at least one anticancer agent comprises a KRAS-MAPK inhibitor to the subject.

Embodiment IX. The method of Embodiment VIII, wherein the KRAS-MAPK inhibitor is AMG510 (Sotorasib), MRTX849 (Adagrasib), or MRTX-1133.

Embodiment X. A method of treating a subject having NEPC, PDAC, PNETs, or NECs the method comprising:
(a) determining whether a KRAS mutation and/or a Trp53 mutation is present or absent in a biological sample taken from the subject; and
(b) administering a therapeutically effective amount of ESK981 to the subject if a KRAS mutation and/or a Trp53 mutation is present in the biological sample.

Embodiment XI. A method, comprising administering a therapeutically effective amount of ESK981 to a subject in need thereof, wherein:
(a) the subject has NEPC, PDAC, PNETs, or NECs; and
(b) the PDAC, PNETs, or NECs is characterized as having a KRAS mutation and/or a Trp53 mutation.

Embodiment XII. The method of Embodiments X or XI further comprising administering a KRAS-MAPK inhibitor to the subject.

Embodiment XIII. The method of Embodiment XII, wherein the KRAS-MAPK inhibitor is AMG510 (Sotorasib), MRTX849 (Adagrasib), or MRTX-1133.

Embodiment XIV. A method of identifying whether a subject having NEPC, PDAC, PNETs, or NECs as a candidate for treatment with ESK981, the method comprising:
(a) determining whether a KRAS mutation and/or a Trp53 mutation is present or absent in a biological sample taken from the subject; and
(b) identifying the subject as being a candidate for treatment if the KRAS mutation and/or a Trp53 mutation is present; or
(c) identifying the subject as not being a candidate for treatment if the KRAS mutation and/or a Trp53 mutation is absent.

Embodiment XV. A method of predicting treatment outcome in a subject having NEPC, PDAC, PNETs, or NECs, the method comprising determining whether a KRAS mutation and/or a Trp53 mutation is present or absent in a biological sample taken from the subject, wherein:
(a) the presence of a KRAS mutation and/or a Trp53 mutation in the biological sample indicates that administering ESK981 to the subject will likely cause a favorable therapeutic response; and
(b) the absence of a KRAS mutation and/or a Trp53 mutation in the biological sample indicates that administering ESK981 to the subject will likely cause an unfavorable therapeutic response.

Embodiment XVI. The method of any one of Embodiments IX-XIII, wherein the KRAS mutation is Kras^{G12D}.

Embodiment XVII. The method of any one of Embodiments IX-XIV, wherein the Trp53 mutation is Trp53^{R172H}.

The disclosure provides the following particular embodiments.

Embodiment 1. A method of treating cancer in a subject in need thereof, the method comprising administering to the subject a therapeutically effective amount of 13-isobutyl-4-methyl-10-(pyrimidin-2-ylamino)-1,2,4,7,8,13-hexahydro-6H-indazolo[5,4-a]pyrrolo[3,4-c]carbazol-6-one (ESK981), or pharmaceutically acceptable salt or solvate thereof, wherein the cancer is neuroendocrine prostate cancer (NEPC), pancreatic ductal adenocarcinoma (PDAC), pancreatic neuroendocrine tumors (PNETs), pancreatic neuroendocrine carcinomas (NECs), colorectal cancer (CRC), or non-small cell lung cancer (NSCLC).

Embodiment 2. The method of Embodiment 1, wherein the cancer is NEPC.

Embodiment 3. The method of Embodiment 1, wherein the cancer is PDAC.

Embodiment 4. The method of Embodiment 1, wherein the cancer is PNETs.

Embodiment 5. The method of Embodiment 1, wherein the cancer is NECs.

Embodiment 6. The method of Embodiment 1, wherein the cancer is CRC.

Embodiment 7. The method of Embodiment 1, wherein the cancer is NSCLC.

Embodiment 8. The method of any one of Embodiments 1-7, wherein ESK981, or pharmaceutically acceptable salt or solvate thereof, is:
(i) a crystalline para-toluenesulfonic acid (pTSA) salt having an ESK981:pTSA: ratio of 1:1, characterized as having an x-ray powder diffraction (XRPD) pattern comprising peaks at 5.37, 6.79, 13.64, 22.58, and 25.54 degrees 2Θ using Cu Kα radiation, wherein the 2Θ values are ±0.2 degrees 2Θ (ESK981 pTSA-A₁);
(ii) a crystalline pTSA salt having an ESK981:pTSA ratio of 1:2, characterized as having an XRPD pattern comprising peaks at 5.63, 8.48, 12.46, 18.21, and 23.95 degrees 2Θ using Cu Kα radiation, wherein the 2Θ values are ±0.2 degrees 2Θ (ESK981 pTSA-A₂);
(iii) a crystalline pTSA salt having an ESK981:pTSA ratio of 1:2, characterized as having an XRPD pattern comprising peaks at 3.75, 5.63, 8.40, 11.31, and 15.12 degrees 2Θ using Cu Kα radiation, wherein the 2Θ values are ±0.2 degrees 2Θ (ESK981 pTSA-B₂);
(iv) a crystalline pTSA salt having an ESK981:pTSA ratio of 1:2, characterized as having an XRPD pattern comprising peaks at 5.72, 8.49, 11.37, 13.26, and 16.95 degrees 2Θ using Cu Kα radiation, wherein the 2Θ values are ±0.2 degrees 2Θ (ESK981 pTSA-C₂); or
(v) a crystalline acetic acid (CH₃CO₂H) salt having an ESK981:CH₃CO₂H ratio of 1:2, characterized as having an XRPD pattern comprising peaks at 6.17, 6.44, 14.36, 20.69, and 26.09 degrees 2Θ using Cu Kα radiation, wherein the 2Θ values are ±0.2 degrees 2Θ (ESK981 AA).

Embodiment 9. The method of any one of Embodiments 1-8, further comprising administering a therapeutically effective amount of an anticancer agent to the subject in combination with ESK981, or pharmaceutically acceptable salt or solvate thereof.

Embodiment 10. The method of Embodiment 9, wherein the anticancer agent comprises a chemotherapeutic agent, an immune checkpoint inhibitor, and/or radiation therapy.

Embodiment 11. The method of Embodiment 9, wherein the anticancer agent comprises a RAS inhibitor.

Embodiment 12. The method of Embodiment 11, wherein the RAS inhibitor is a pan-RAS inhibitor.

Embodiment 13. The method of Embodiment 11, wherein the RAS inhibitor is a RAS^{WT/X} inhibitor.

Embodiment 14. The method of Embodiment 11, wherein the RAS inhibitor is a KRAS inhibitor.

Embodiment 15. The method of Embodiment 14, wherein the KRAS inhibitor is a KRAS^{G12C} inhibitor.

Embodiment 16. The method of Embodiment 14, wherein the KRAS inhibitor is a KRAS^{G12D} inhibitor.

Embodiment 17. The method of Embodiment 14, wherein the KRAS inhibitor is a KRAS^{G12V} inhibitor.

Embodiment 18. The method of Embodiment 14, wherein the KRAS inhibitor is a KRAS^{G13C} inhibitor.

Embodiment 19. The method of Embodiment 14, wherein the KRAS inhibitor is a KRAS^{Q61H} inhibitor.

Embodiment 20. The method of Embodiment 11, wherein the RAS inhibitor is MRTX849 (Adagrasib), AMG 510 (Sotorasib), BBO-8520, BBO-8956, D3S-001 (Elisrasib), GDC-6036 (Divarasib), D-1553 (Garsorasib), GH35, JAB-21822 (Glecirasib), IBI351/GFH925 (Fulzerasib), LY3499446, MK-1084, LY3537982 (Olomorasib), JDQ443 (Opnurasib), RMC-4998, RMC-6291 (Elironrasib), ASP3082, HRS-4642, MRTX1133, RMC-9805 (Zoldonrasib), RMC-5127, ACBI3, BI-2493, BI-2865, RMC-6236 (Daraxonrasib), RMC-7977, BEBT-607, BI 1823911, BPI-421286, FMC-376, GEC255, HBI-2438, HS-10370, JNJ-74699157, YL-15293, GFH357/VS-7375, INCB161734, JAB-22000, LY3962673, QTX3046, TSN1611, RMC-8839, RMC-0708, BI 3706674, JAB-23E73, LY4066434, PF-07934040, QTX3034, GFH547, YL-17231, BPI-585725, or ERAS-4001.

Embodiment 21. The method of Embodiment 20, wherein the RAS inhibitor is AMG510 (Sotorasib), MRTX849 (Adagrasib), or MRTX-1133.

Embodiment 22 A method of treating a subject having NEPC, PDAC, PNETs, NECs, CRC, or NSCLC, the method comprising:
(a) determining whether a KRAS mutation and/or a Trp53 mutation is present or absent in a biological sample taken from the subject; and
(b) administering a therapeutically effective amount of ESK981, or pharmaceutically acceptable salt or solvate thereof, to the subject if a KRAS mutation and/or a Trp53 mutation is present in the biological sample.

Embodiment 23. A method, comprising administering a therapeutically effective amount of ESK981, or pharmaceutically acceptable salt or solvate thereof, to a subject in need thereof, wherein:
(a) the subject has NEPC, PDAC, PNETs, NECs, CRC, or NSCLC; and
(b) the NEPC, PDAC, PNETs, NECs, CRC, or NSCLC is characterized as having a KRAS mutation and/or a Trp53 mutation.

Embodiment 24. The method of Embodiments 22 or 23, wherein the cancer is NEPC.

Embodiment 25. The method of Embodiments 22 or 23, wherein the cancer is PDAC.

Embodiment 26. The method of Embodiments 22 or 23, wherein the cancer is PNETs.

Embodiment 27. The method of Embodiments 22 or 23, wherein the cancer is NECs.

Embodiment 28. The method of Embodiments 22 or 23, wherein the cancer is CRC.

Embodiment 29. The method of Embodiments 22 or 23, wherein the cancer is NSCLC.

Embodiment 30. The method of any one of Embodiments 22-29, wherein the KRAS mutation is Kras^{G12D}.

Embodiment 31. The method of any one of Embodiments 22-29, wherein the KRAS mutation is Kras^{G12C}.

Embodiment 32. The method of any one of Embodiments 22-29, wherein the KRAS mutation is Kras^{G12V}.

Embodiment 33. The method of any one of Embodiments 22-29, wherein the KRAS mutation is Kras^{G13C}.

Embodiment 34. The method of any one of Embodiments 22-29, wherein the KRAS mutation is Kras^{Q61H}.

Embodiment 35. The method of any one of Embodiments 22-29, wherein the Trp53 mutation is Trp53^{R172H}.

Embodiment 36. The method of any one of Embodiments 22-35, wherein ESK981, or pharmaceutically acceptable salt or solvate thereof, is ESK981 pTSA-A₁, ESK981 pTSA-A₂, ESK981 pTSA-B₂, ESK981 pTSA-C₂, or ESK981 AA.

Embodiment 37. The method of any one of Embodiments 22-36, further comprising administering a therapeutically effective amount of an anticancer agent to the subject in combination with ESK981, or pharmaceutically acceptable salt or solvate thereof.

Embodiment 38. The method of Embodiment 37, the anticancer agent comprises a RAS inhibitor.

Embodiment 39. The method of Embodiment 38, wherein the KRAS mutation is Kras^{G12D} and the RAS inhibitor is a KRAS^{G12D} inhibitor.

Embodiment 40. The method of any one of Embodiments 22-43, wherein the KRAS mutation is Kras^{G12C} and the RAS inhibitor is a KRAS^{G12C} inhibitor.

Embodiment 41. The method of Embodiment 38, wherein the KRAS mutation is Kras^{G12V} and the RAS inhibitor is a Kras^{G12V} inhibitor.

Embodiment 42. The method of Embodiment 38, wherein the KRAS mutation is Kras^{G13C} and the RAS inhibitor is a Kras^{G13C} inhibitor.

Embodiment 43. The method of Embodiment 38, wherein the KRAS mutation is Kras^{Q61H} and the RAS inhibitor is a Kras^{Q61H} inhibitor.

Embodiment 44. The method of Embodiment 38, wherein the RAS inhibitor is the RAS inhibitor is MRTX849 (Adagrasib), AMG 510 (Sotorasib), BBO-8520, BBO-8956, D3S-001 (Elisrasib), GDC-6036 (Divarasib), D-1553 (Garsorasib), GH35, JAB-21822 (Glecirasib), IBI351/GFH925 (Fulzerasib), LY3499446, MK-1084, LY3537982 (Olomorasib), JDQ443 (Opnurasib), RMC-4998, RMC-6291 (Elironrasib), ASP3082, HRS-4642, MRTX1133, RMC-9805 (Zoldonrasib), RMC-5127, ACBI3, BI-2493, BI-2865, RMC-6236 (Daraxonrasib), RMC-7977, BEBT-607, BI 1823911, BPI-421286, FMC-376, GEC255, HBI-2438, HS-10370, JNJ-74699157, YL-15293, GFH357/VS-7375, INCB161734, JAB-22000, LY3962673, QTX3046, TSN1611, RMC-8839, RMC-0708, BI 3706674, JAB-23E73, LY4066434, PF-07934040, QTX3034, GFH547, YL-17231, BPI-585725, or ERAS-4001

Embodiment 44. The method of Embodiment 44, wherein the RAS inhibitor is AMG510 (Sotorasib), MRTX849 (Adagrasib), or MRTX-1133.

Embodiment 45. A method of identifying whether a subject having NEPC, PDAC, PNETs, NECs, CRC, or NSCLC is a candidate for treatment with ESK981, or pharmaceutically acceptable salt or solvate thereof, the method comprising:
(a) determining whether a KRAS mutation and/or a Trp53 mutation is present or absent in a biological sample taken from the subject; and
(b) identifying the subject as being a candidate for treatment if the KRAS mutation and/or a Trp53 mutation is present; or
(c) identifying the subject as not being a candidate for treatment if the KRAS mutation and/or a Trp53 mutation is absent.

Embodiment 46. A method of predicting treatment outcome in a subject having NEPC, PDAC, PNETs, NECs, CRC, or NSCLC, the method comprising determining whether a KRAS mutation and/or a Trp53 mutation is present or absent in a biological sample taken from the subject, wherein:
(a) the presence of a KRAS mutation and/or a Trp53 mutation in the biological sample indicates that administering ESK981, or pharmaceutically acceptable salt or solvate thereof, to the subject will likely cause a favorable therapeutic response; and
(b) the absence of a KRAS mutation and/or a Trp53 mutation in the biological sample indicates that administering ESK981, or pharmaceutically acceptable salt or solvate thereof, to the subject will likely cause an unfavorable therapeutic response.

Embodiment 47. The method of Embodiments 45 or 46, wherein the KRAS mutation is Kras^{G12D}.

Embodiment 45. The method of Embodiments 45 or 46, wherein the KRAS mutation is Kras^{G12C}.

Embodiment 46. The method of Embodiments 45 or 46, wherein the KRAS mutation is Kras^{G12V}.

Embodiment 47. The method of any one of Embodiments 22-43, wherein the KRAS mutation is Kras^{G13C}.

Embodiment 48. The method of Embodiments 45 or 46, wherein the KRAS mutation is Kras^{Q61H}.

Embodiment 49. The method of Embodiments 45 or 46, wherein the Trp53 mutation is Trp53^{R172H}.

Embodiment 50. A kit for carrying out the methods of Embodiments 1-44 the kit comprising ESK981, or a pharmaceutically acceptable salt and/or solvate thereof, and a label with instructions for how to administer ESK981, or a pharmaceutically acceptable salt and/or solvate thereof, to a subject.

Embodiment 51. The kit of Embodiment 50, wherein the label is approved by at least one regulatory health agency.

Embodiment 52. The kit of Embodiment 38, wherein the regulatory health agency is United States Food and Drug Administration (FDA), the European Medicines Agency (EMA), the China Food and Drug Administration (CFDA), or the Japanese Ministry of Health Labor and Welfare (MHLW).

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### General Methods

### Mouse Strains

Ptfia-Cre, Ptfia-Cre; lsl-Kras^{G12D} (KC), and Ptfia-Cre; lsl-Kras^{G12D}; p53^{R172H/+} (KPC) mice were provided by Marina Pasca di Magliano at the University of Michigan. Conditionally floxed Pikfyve (Pikfyve^{f/f}) mice were purchased from Jackson labs. PCR genotyping for Ptfia-Cre, Kras^{G12D}, p53^{R172H/+} and Pikfyve^{f/f} alleles, from DNA isolated from mouse tails, was performed using standard methodology. Littermate controls were systematically used in all experiments, and the sex ratios for each cohort were balanced. All animals were housed in a pathogen-free environment, and all procedures were performed in accordance with requirements of the University of Michigan Institutional Animal Care & Use Committee (IACUC).

### Cell Lines, Antibodies, and Compounds

Most cell lines were originally obtained from the American Type Culture Collection (ATCC). 7940B was provided by Greggory Beatty, M.D., Ph.D. at Perlman School of Medicine at the University of Pennsylvania. The iKRAS 9805 cell line was generated by Dr. Marina Pasca di Magliano, Ph.D., at the University of Michigan. The UM PDAC primary cell cultures (UM-2, UM-19) were obtained from surgically resected samples and established through murine xenograft. KPC-1344 was derived from a KPC mouse in-house by dissociating tumors manually with a sterile blade and then treating them with 1 mg/mL collagenase II (ThermoFisher Scientific cat. no. 17101-015) and 1 mg/mL DNase (Sigma-Aldrich, cat. no. 10104159001) for 30 minutes with shaking at 37° C. The cells were then strained using a MACS SmartStrainer (30 μM) (Miltenyi Biotec cat no. 130-110-915) and rinsed with PBS prior to culturing. All cells were grown in Gibco DMEM+10% FBS (ThermoFisher). All cell lines were genotyped to confirm their identity at the University of Michigan Sequencing Core and tested biweekly for Mycoplasma contamination.

### Histopathologic Analyses

Pathologists conducted a detailed histopathological evaluation of murine pancreatic tissues on 4 μm thick H&E-stained formalin fixed paraffin embedded (FFPE) sections. The examination involved checking all harvested pancreas samples for the percentage prevalence of normal pancreas, pancreatic intraepithelial neoplasia (PanIN)—either high and low grade, and lesions with atypia or frank evidence of pancreatic ductal adenocarcinoma. The samples were then classified under these three categories, and the results were tabulated. Finally, the pathologists reached a consensus to determine the final percentage prevalence.

### BaseScope

The BaseScope™ VS Reagent Kit (Cat. No. 323700; Advanced Cell Diagnostics, Newark, CA), which is used to identify short targets and splice variants, was employed to demonstrate Pikfyve on whole mouse pancreatic tissues. The reagent kit was used with the Discovery Ultra automated IHC/ISH slide staining systems by Ventana Medical Systems on a validated protocol utilizing BaseScope™ VS Detection Reagents (Cat. No. 323710), RNAscope Universal VS Sample Preparation Reagents v2 (Cat. No. PN323740), and RNAscope VS Accessory Kit (320630). BaseScope™ VS Probe—BA-Mm-Pikfyve-E6-3zz-st-C1, Mus musculus phosphoinositide kinase FYVE type zinc finger containing (Pikfyve) transcript variant 2 mRNA targeting exon 6 complimentary to the target mRNA was employed (Cat. No. 1300097-C1; accession #NM_011086.2, nucleotides 633-771) for the assay as test probe. BaseScope™ VS Positive Control Probe-Mm-PPIB-3ZZ-Mus musculus peptidylprolyl isomerase B (Ppib)mRNA (Cat. No701079) and BaseScope™ VS Negative Control Probe-DapB-3ZZ (Cat No. 701019) were used as positive and negative controls, respectively.

All slides were examined for positive signals in lesions and background benign pancreas by pathologists. The RNA in situ hybridization signal was identified as red, punctate dots, and the expression level was scored as follows: 0=no staining or <1 dot per 10 cell (at 40× magnification), 1=1 dot per cell (visible at 20/40×), 2=2-3 dots per cell, 3=4-10 per cell (<10% in dot clusters) visible at 20×, and 4=>10 dots per cell (>10% in dot clusters) visible at 20×. A cumulative RNA ISH product score was calculated for each evaluable tissue core as the sum of the individual products of the expression level (0 to 4) and percentage of cells [0 to 100; ie, (A %×0)+(B %×1)+(C %×2)+(D %×3)+(E %×4); total range=0 to 400]

### Immunohistochemistry

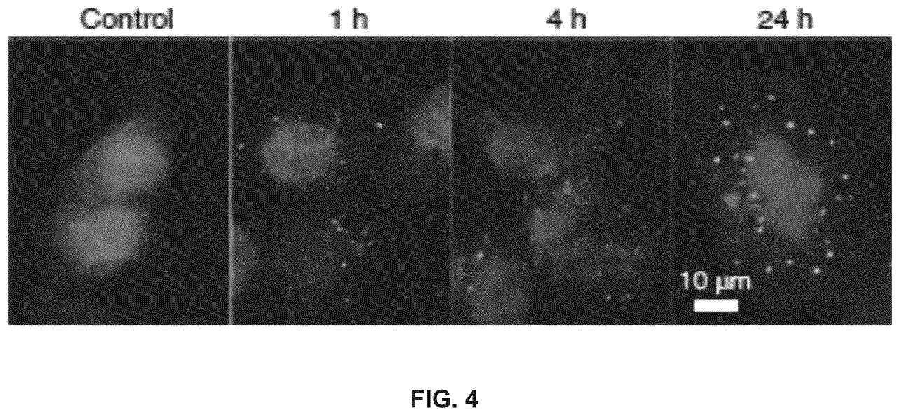

Immunohistochemistry was performed on formalin-fixed paraffin-embedded 4 mM sections of mouse or xenograft tissues. Slides were deparaffinized in xylene, followed by serial hydration steps in ethanol (100%, 95%, 70%) and water for 4 minutes each. Antigen retrieval was performed by boiling slides in citrate buffer (pH 6). Endogenous tissue peroxidase activity was blocked by 3% H₂O₂ for 1 hour. Slides were blocked in 10% goat serum for 1 hour. The slides were then incubated in the primary antibody (CK19, ab52625) at a dilution ratio of 1:200. The quantification in FIG. 4L was performed using Fiji. After being converted to 8-bt, the images underwent color deconvolution. The thresholds were set at for all images. The ratio of brown signal to total signal was calculated to be CK19% positive area. Non-pancreas areas (i.e. spleen) were excluded from the analysis. Visualization of staining was done per the manufacturer's protocol (Vector Laboratories, cat. no. SK-4100). Following DAB staining, slides were dehydrated in ethanol (70%, 95%, 100%, 6 minutes each), xylene (15 minutes), and mounted using EcoMount (Thermo Fisher, cat. no. EM897L).

### In Vivo Tumor Studies

All animal experiments were conducted in accordance with the Office of Laboratory Animal Welfare and approved by the University of Michigan IACUC.

### Subcutaneous Tumor Studies

For xenograft studies, 6-10-week-old CB17 severe combined immunodeficiency (SCID) mice obtained from the University of Michigan breeding colony were used. For syngeneic studies, 6-10-week-old C57BL6 mice obtained from Jackson Laboratories were used. Subcutaneous tumors were established at both sides of the dorsal flank of the mice by injecting 1×10⁶ cells in 100 mL of 50:50 Matrigel and serum-free media. Tumors were measured 2-3 times per week using digital calipers following the formula (π/6) (L×W2), where L=length and W=width of the tumor. At the end of the studies, mice were sacrificed, and tumors extracted and weighed.

### Orthotopic Tumor Study

The 7940B orthotopic model was established according to previously described protocols. Briefly, 50,000 cells were implanted directly into the pancreas of C57BL6 mice (Jackson Laboratories). Tumors were established for 11 days prior to treatment with the indicated conditions. Mice were sacrificed at 3 weeks of treatment, and tumors were weighed and preserved for further analyses.

### In Vivo Apoptosis Evaluation Using TUNEL Staining

Terminal dUTP Nick End Labeling (TUNEL) staining was performed with an In Situ Cell Death Detection Kit (TMR Red #12156792910; Roche Applied Science) following the manufacturer's instructions. Briefly, fixed sections were permeabilized using Triton X-100 followed by PBS washing. The labelling reaction was performed at 37° C. for 1 hour by addition of the reaction buffer containing enzymes. Images were acquired using a Zeiss Axiolmager M1 microscope.

### Cellular Thermal Shift Assay (CETSA)

CETSA was performed according to previously described protocols⁷¹. Briefly, 7940B cells were seeded overnight and were subsequently treated with DMSO, ESK981 (1000 nM), or apilimod (1000 nM) for 2 hours. Cells were then harvested and made into single-cell suspensions of 1×10⁶ cells each in 50 mL of PBS containing protease inhibitors. The suspensions were then subjected to heating and cooling cycles (two cycles of 3-minute heating followed by 3-minute cooling at room temperature) using a thermal cycler. Cells were then lysed with three cycles of freeze-thawing in liquid nitrogen. Lysates were then analyzed using immunoblot analysis as previously described.

### Cell Viability Assays and Synergy Assays

Cells were plated into 96-well plates and incubated overnight at 37° C. in 5% CO₂. The following day, a serial dilution of the indicated compounds was prepared in culture medium and added to the plate. The cells were then further incubated for 5 days (experiments involving MRTX1133 or trametinib) or 7 days (all other experiments). Subsequently, the CellTiter-Glo assay (Promega), was then performed according to the manufacturer's instructions. The luminescence signal was acquired using an Infinite M1000 Pro plate reader (Tecan), and the data were analyzed using GraphPad Prism 10 (GraphPad Software Inc.).

To determine the synergism of two different compounds using viability assays, cells were treated with the indicated combinations of the drugs for 5 days prior to performing the CellTiter-Glo assay as described above. These experiments were performed with 5 biological replicates each with 10 wells of untreated internal controls for each plate used in each experiment which were used for normalization between plates. The data were then expressed as percent inhibition relative to baseline, and the presence of synergy was determined by the Bliss method using the SynergyFinder+ web application.

### Immunoblots

Cell lysates were prepared in RIPA buffer (ThermoFisher Scientific) supplemented with Halt™ Protease and Phosphatase Inhibitor Cocktail (ThermoFisher Scientific). Total protein was measured by DC™ Protein Assay Kit II (BIO-RAD). An equal amount of protein was resolved in NuPAGE™ 3 to 8%, Tris-Acetate Protein Gel (ThermoFisher Scientific) or NuPAGE™ 4 to 12%, Bis-Tris Protein Gel (ThermoFisher Scientific), blocked with 5% nonfat dry milk in TBS-T and blotted with primary antibodies overnight. Following incubation with HRP-conjugated secondary antibodies, membranes were imaged on an Odyssey CLx Imager (LiCOR Biosciences).

### Autophagic Flux Probe Generation and Assay

Generation of the autophagic flux probe in 7940B, Panc 04.03, and iKRAS was done according to the original author's instructions. Kaizuka, T. et al., Mol Cell 64, 835-849, doi:10.1016/j.molcel.2016.09.037 (2016). Briefly, cells were infected with pMRX-IP-GFP-LC3-RFP-LC3DG, which was a gift from Noboru Mizushima (Addgene plasmid #84572; http://n2t.net/addgene:84572; RRID:Addgene_84572). Following puromycin selection, single-cell clones were expanded and genotyped to ensure the absence of homologous recombination between two LC3 fragments during retrovirus integration.

15,000 cells were seeded in 96-well plates. After overnight incubation, cells were treated with the indicated compounds for 24 hours. For assays assessing co-treatment of autophagy inhibitors (i.e., apilimod, ESK981, chloroquine) with autophagy inducers (torin-1, trametinib, MRTX1133), the autophagy inhibitor was added 4 hours prior to the inducer. For assays using iKRAS, cells were seeded with or without doxycycline, as indicated, and then treated with compounds in a similar fashion. Fluorescent signals were detected using the Infinite M1000 Pro plate reader (Tecan).

### Confluence-Based Proliferation Assays (Incucyte)

Cells were seeded in a clear 96-well plate overnight prior to treatment. Upon treatment with indicated compounds, plates were incubated in an Incucyte S3 2022 Rev1 (Sartorious), with 10× images taken every 4 hours, and well confluence was analyzed to assess for proliferation.

### Oxygen Consumption Assays

Oxygen consumption rates were determined using the Seahorse XF Glycolytic Rate Assay (Agilent) according to the manufacturer's protocol. Briefly, 15,000 (7940B) or 25,000 (Panc 04.03) cells were seeded in an Agilent XF96 Cell Culture Microplate 16 hours prior to treatment. Cells were treated with AP, ESK, CQ, or BAF as indicated for 8 hours. Immediately prior to the assay, cells were washed and then incubated in XF DMEM medium (pH 7.4, Agilent) with 1 mM pyruvate, 2 mM glutamine, and 10 mM glucose. The assay was conducted on an XF96 Extracellular Flux Analyzer (Agilent), and the OCR was calculated using Wave (version 2.6, Agilent). OCR was normalized to cell number with the CyQUANT NF Cell Proliferaiton Assay (Invitrogen) according to the manufacturer's instructions.

Real-time monitoring of basal oxygen consumption rate was performed using a Resipher (Lucid Scientific). 15,000 7940B cells were seeded in 50 Q1 of medium in a clear 96-well plate 16 hours prior to treatment. Immediately following treatment with an additional 50 μl of medium (for a total of 100 μl), OCR monitoring was started by placing the Resipher device on the cells, which was incubated at 37° C. and 5% CO₂ for 24 hours.

### Metabolic CRISPR Screen

The Human CRISPR Metabolic Gene Knockout library was a gift from David Sabatini (Addgene #110066). To achieve at least 1000-fold coverage of the library while culturing, 75×10⁶ MIA PaCa-2 cells were seeded at a density of 5×10⁵ cells/mL in 6-well plates containing 2 mL of DMEM, 8 mg/mL polybrene, and the CRISPR screen library virus. Spin infection was caried out by centrifugation at 1200 g for 45 minutes at 37° C. After 24-hour incubation, the media was replaced with fresh DMEM. After a subsequent 24-hour incubation, cells were transferred to T-150 flasks (at a density of 3 wells into 1 T150 flask) containing 20 mL of DMEM containing puromycin at 2 mg/mL. After 3 days of selection, cells were seeded into sixteen total T-150 flasks at a density of 5×10⁶ cells/flask in 20 mL of DMEM containing either DMSO or 200 nM of apilimod. Cells were passaged every 3-4 days and re-seeded back to the original cell density. After 14 days, a pool of 15 million cells from each condition were harvested for genomic DNA (gDNA) isolation using the DNeasy blood and tissue kit (Qiagen) according to the manufacturer's protocol.

For each condition, sgRNA was amplified from 50 mg gDNA using Herculase II Fusion DNA Polymerase (Agilent Technologies), column purified using Select-a-Size DNA Clean & Concentrator kit (Zymo Research), and then gel-purified using 6% Novex TBE gel (Thermo), followed by isolation from the gels with Gel Breaker Tubes and Gel Filters (BioChain). The resulting PCR products then underwent end-repair and A-tail addition followed by New England Biolabs (NEB) adapter ligation. The final library was prepared by enriching adapter-ligated DNA fragments using 2×KAPA HiFi HotStart mix and NEB dual code barcode following the manufacter's protocol. The libraries were then sequenced on an Illumina NovaSeq 6000 (paired-end, 300 cycles).

Reads were trimmed to the bare sgRNA sequence using cutadapt 4.1. Paired-end mates were trimmed separately using a sequence 5′-adjacent to the sgRNA position within the vector requiring a minimum match of 18 bases to the sequence and followed by truncation to 20 bases (relevant cutadapt command parameters: -m 18 -O 18 -l 20—discard-untrimmed). Trimmed reads were then combined and aligned using bowtie2 2.4.5 to a reference built from each sgRNA in the library flanked by vector sequences. See, e.g., Langmead, B. & Salzberg, S. L. Nat Methods 9, 357-359, doi:10.1038/nmeth.1923 (2012).

The bowtie2 parameter --norc was used to prevent reverse compliment alignment. Counting was then performed using MAGeCK 0.5.9.5.

sgRNAs with less than 100 counts in the initial dataset were removed from downstream analysis. Genes targeted by fewer than 6 distinct sgRNAs following this filtering were likewise removed. Downstream analyses, including calculation of sgRNA depletion/enrichment scores, gene depletion/enrichment scores, and selective dependency, were done according to previously described methods. Briefly, normalized sgRNA abundances were calculated by adding a pseudocount of one and then normalized to the total counts of each sample. The sgRNA enrichment/depletion scores were calculated as log 2 fold change between the final and initial populations, and the gene scores were calculated as the average log 2 fold change of the sgRNAs targeting that gene. To calculate selective essentiality scores, we first scaled gene scores using the medians of nontargeting sgRNAs and sgRNAs targeting core essential genes as references (0 and −1, respectively). Selective essential genes were then identified by taking the Z-scored difference between the scaled apilimod and DMSO gene scores. Plots were generated using ggplot2 (version 3.4.4).

RNA Isolation and Quantitative Real-Time PCR (qPCR)

Total RNA was isolated from cells using the miRNeasy kit (Qiagen), and cDNA was synthesized from 1000 ng of total RNA using Maxima First Strand cDNA Synthesis Kit for RT-qPCR (Thermo Fisher Scientific). Quantitative real-time PCR was performed in triplicates using standard SYBR green reagents and protocols on a QuantStudio 5 Real-Time PCR system (Applied Biosystems). The target mRNA expression was quantified using the ΔΔCt method and normalized to ACTB (human) or Actb (murine) expression. All primers were designed using Primer 3 (http://frodo.wi.mit.edu/primer3/) and synthesized by Integrated DNA Technologies (Coralville).

### RNA-Seq and Analysis

RNA-seq libraries were prepared using 800 ng of total RNA. Ribosomal RNA were removed by enzymatic digestion of the specific probe-bound duplex rRNA, and then fragmented to around 200-300 bp with heat in fragmentation buffer (KAPA RNA Hyper+RiboErase HMR, Roche). Double-stranded cDNA was then synthesized by reverse transcription and underwent end-repair and ligation using New England Biolabs (NEB) adapters. Final library preparation was then prepared by amplification with 2×KAPA HiFI HotStart mix and NEB dual barcode. Library quality was measured on an Agilent 2100 Bioanalyzer (DNA 1000 chip) for concentration and product size. Paired-end libraries were sequenced with the Illumina NovaSeq, (2×151 nucleotide read length) with sequence coverage to 30-40 million paired reads. Reads were demultiplexed using Illumina's bcl2fastq conversion software v2.20. Transcripts were quantified by the alignment-free approach kallisto using index generated from mouse reference genome (mm10) and then summed to obtain gene level counts. Differential analysis was performed using limma-voom after TMM-normalization of gene level counts with calcNormFactors of edgeR. Genes with mean Transcripts Per Million (TPM) less than 1 in both control and treatment groups were considered as lowly expressed genes and excluded for differential analysis. Enrichment of Hallmark and Reactome gene sets downloaded from MSigDB were examined with fgsea using genes ranked by log FC estimated from limma as input.

### Generation of CRISPRi-Mediated Knockdown Cell Lines

sgRNA sequences used were taken from previously validated Perturb-seq library. The backbone, pLV hU6-sgRNA hUbC-dCas9-KRAB-T2a-Puro, was a gift from Charles Gersbach (Addgene plasmid #71236; http://n2t.net/addgene:71236; RRID: Addgene_71236). The generated plasmids were then expanded, verified by Sanger sequencing, and packaged into lentiviruses by the University of Michigan Vector Core. Cells were seeded, infected with viruses along with polybrene (10 mg/mL), and then selected with puromycin (2 mg/mL for MIA PaCa-2, 5 mg/mL for PANC-1) prior to further analysis. Given the notable impact of PIKFYVE and FASN knockdown on PDAC cells, new CRISPRi knockdown cell lines were generated prior to each experiment.

### ESK981, Trametinib, and MRTX1133 Formulation for In Vivo Studies

ESK981 was added to ORA-PLUS and sonicated until completely dissolved. Trametinib was added to corn oil and sonicated until completely dissolved. Aliquots were frozen at −20° C. to prevent freeze-thaw cycles. MRTX1133 was added to 10% Captisol in 50 mM Citrate (pH=5.0) and sonicated until completely dissolved as previously described. Dissolved MRTX1133 was kept at 4° C. hidden from light for a maximum for 5 days. ESK981 and trametinib were delivered by oral gavage. MRTX1133 was delivered by intraperitoneal (IP) injection.

### Targeted Metabolomics

Polar metabolites from samples treated in biological triplicates were extracted using 80% v/v methanol/water and normalized using protein quantification from an additional sample from each condition. Equal estimated amounts of metabolites were dried using a SpeedVac vacuum concentrator, reconstituted in 50% v/v methanol in water, and analyzed by LC-MS as previously described. Data were analyzed as previously described with the Agilent Masshunter Workstation Quantitative Analysis for QQQ version 10.1 Build 10.1.733.0. No post-detection normalization was performed to avoid assuming linearity of signal. Heatmaps were generated using the Morpheus Matrix Visualization and analysis tool (https://software.broadinstitute.org/morpheus).

### Targeted Lipidomics

Sample preparation: Samples for lipidomics analyses were prepared according to the automatic dual-metabolite/lipid sample preparation workflow described in the Agilent application note 5994-5065EN. Briefly, 1 million cells were washed in PBS and lysed with 1:1 trifluoroethanol/water at room temperature. Lysates were transferred to microcentrifuged tubes, incubated for 10 minutes, and then centrifuged at 250×g for 30 seconds. Samples were then dehydrated under reduced pressure with no heat. Samples were then resuspended in 1:1 trifluoroethanol/water and transferred to a 96-well plate and processed on a Brave Metabolomics sample preparation platform (Agilent Technologies, Inc.) with VWorks protocols to isolate lipids as described (5994-5065EN).

LC-MS/MS analysis: samples were analyzed on an Agilent 1290 Infinity II Bio LC ultra-high performed liquid chromatography (UPLC) system consisting of a high-pressure binary pump, multicolumn thermostat, and a temperature controlled multi-sampler. Samples were analyzed in randomized order on an Agilent 649C triple quadrupole mass spectrometer equipped with an Agilent Jet Stream Dual ESI ion source. Specifically, samples were analyzed with the reverse phase LC-MS/MS method reported in the Agilent application note 5994-3747EN. After acquisition, datasets were processed with MassHunter Quantitative analysis software and subsequently imported into Mass Profiler Professional (MPP) for chemometric analysis. No post-detection normalization was performed to avoid assuming linearity of signal.

Changes in lipid class abundance in 7940B cells upon treatment with apilimod (100 nM) or ESK981 (1000 nM) relative to treatment with DMSO were estimated from linear mixed models with random intercepts to adjust for the baseline differences across the lipid classes. A separate model for each treatment (apilimod or ESK981) comparison against DMSO is built using the R package lme4 (version 1.1-35.1).

### Statistics and Reproducibility

No data were excluded from the analyses. No statistical methods were used to predetermine sample sizes. For all in vivo experiments, animals were randomly assigned into treatment cohorts. Tumor measurements were performed by digital caliper in a blinded manner. For all in vitro experiments, cells were seeded from the same pool, and, thus, there was no requirement for randomization. All samples were analyzed equally and simultaneously to eliminate bias. All error bars indicate +/−SD unless otherwise indicated. All statistics comparing two groups were performed using unpaired two-tailed t-tests unless otherwise indicated. All statistics comparing more than two groups were done using an ANOVA with Dunnett's multiple comparison test, using the indicated group as a control, unless otherwise indicated. All statistics comparing two curves were performed using a two-way ANOVA. All statistics comparing four curves were performed using a two-way ANOVA with Dunnett's multiple comparison test, using the indicated curve as a control. GraphPad Prism software (version 10) and R v.4.3.2 were used for statistical calculations. Specific R packages utilized for individual analyses were included in their specific Methods section.

### Example 1

### ESK981 Inhibits Neuroendocrine Prostate Cancer (NEPC) Growth In Vitro and In Vivo

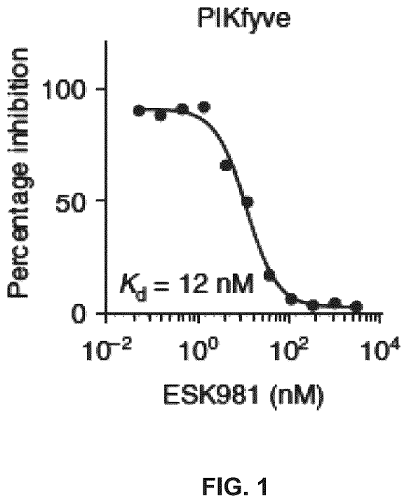

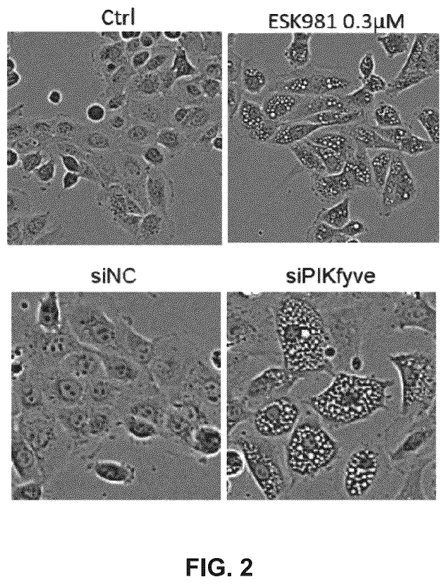

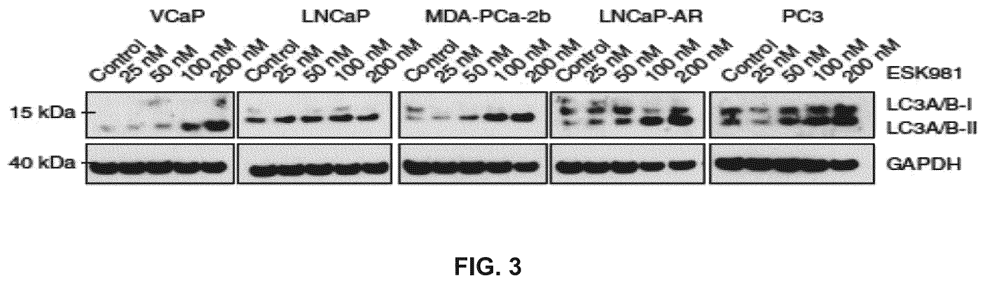

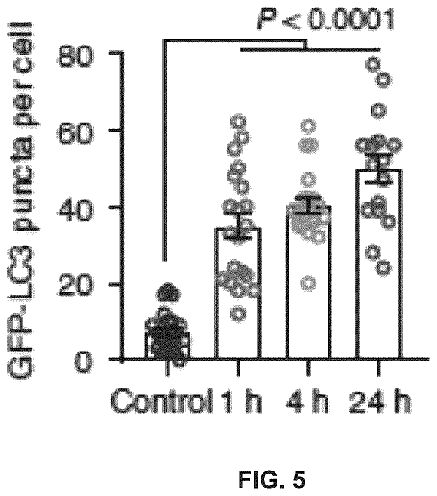

ESK981 inhibits PIKfyve with a dissociation constant (Kd) of 12 nM (FIG. 1). Treatment of DU145 prostate cancer cells engineered to express RFP showed that treatment with 300 nM ESK981 induced vacuolization that phenocopied the morphology of cells transfected with siRNA targeting PIKFYVE (FIG. 2). LC3 lipidation, indicative of autophagosome levels, increased within a short period of treatment with ESK981 (FIGS. 3-5). These data suggest that ESK981 is a potent autophagy inhibitor via targeting of PIKfyve. Qiao et al., Nat Cancer. 2021; 2:978-93.

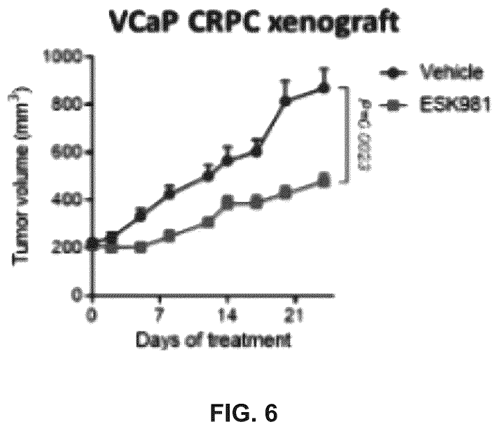

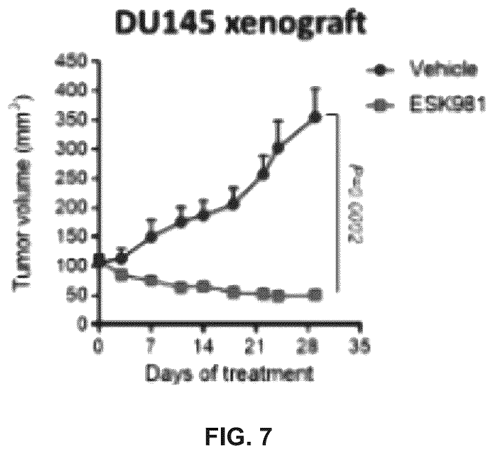

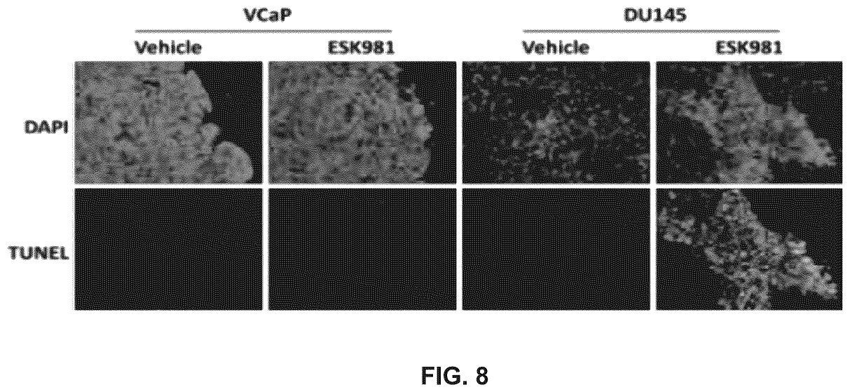

In addition to AR-positive prostate cancer, recent further evaluation of ESK981 suggested that AR-negative prostate cancer might be sensitive to the anti-tumor effects of ESK981. Analysis of tumor growth curves of the AR-positive VCaP castration-resistant prostate cancer (CRPC) xenograft model and compared to AR-negative DU145 xenografts, an enhanced response to ESK981 was observed Levy et al., Nat Rev Cancer. 2017; 17(9):528-42.; Dong et al., Nature Communications. 2010; 1(4):38. When Terminal deoxynucleotidyl transferase sUTP Nick-end Labeling (TUNEL) assays were performed, ESK981, 30 mg/kg, was found to trigger massive cell death in DU145 tumors compared to VCaP (FIG. 8), consistent with DU145 tumor regression observed in the growth curve of FIGS. 6 and 7.

The cytotoxicity of ESK981 in AR-negative xenografts prompted the examination of ESK981 in the highly aggressive neuroendocrine prostate cancer (NEPC) subtype. Most cases of CRPC begin as AR-positive adenocarcinomas that are responsive to AR signaling inhibitors, such as enzalutamide, apalutamide, darolutamide, or abiraterone Schmidt et al., Nat Rev Urol. 2021; 18(4):209-26. However, upon treatment with AR targeting drugs, a significant number of cases develop mechanisms of resistance and become AR-independent, transdifferentiating into a neuroendocrine phenotype Davies et al., Nat Rev Urol. 2018; 15(5):271-86; Epstein et al., Am J Surg Pathol. 2014; 38(6):756-67. PMCID: PMC4112087; Beltran et al., Endocrine-Related Cancer. 2021; 28(8):T67-T78; Wang et al., Nat Rev Urol. 2021; 18(10):581-96. Despite advances in the understanding of NEPC development, treatment options remain limited, with platinum-based chemotherapy as the current first-line treatment for both de novo and treatment-induced NEPC Wang et al., Nat Rev Urol. 2021; 18(10):581-96. The response to first-line chemotherapy in NEPC is short, and most patients die within 12 to 24 months of diagnosis Soundararajan et al., Biochim Biophys Acta Rev Cancer. 2018; 1870(2):229-38. PMCID: PMC6496942.

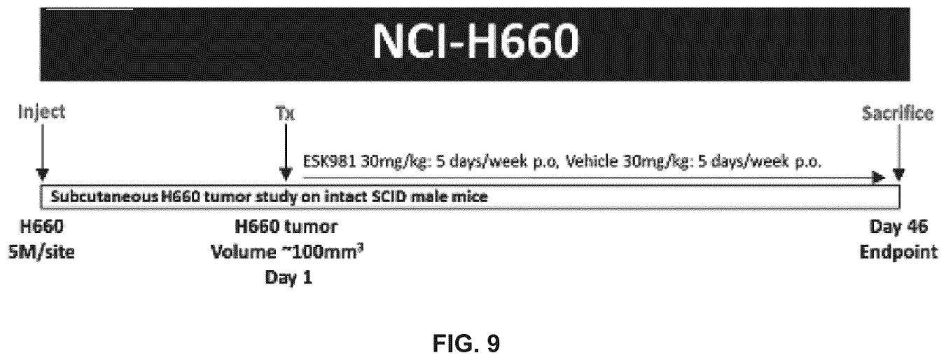

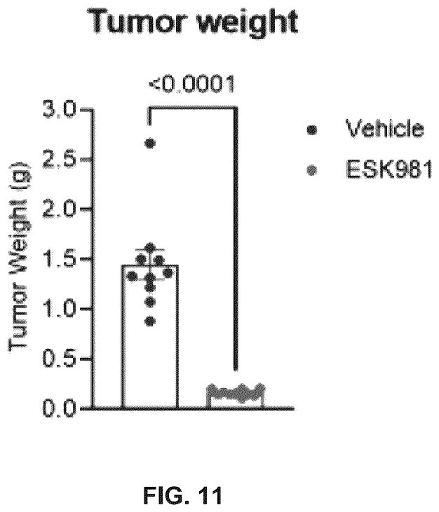

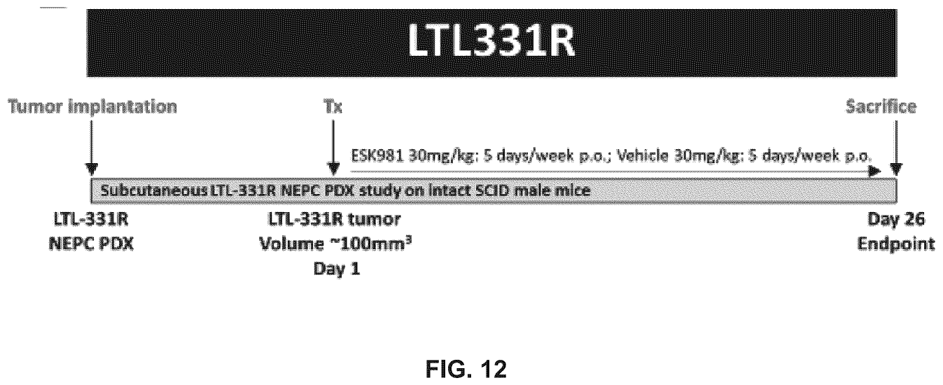

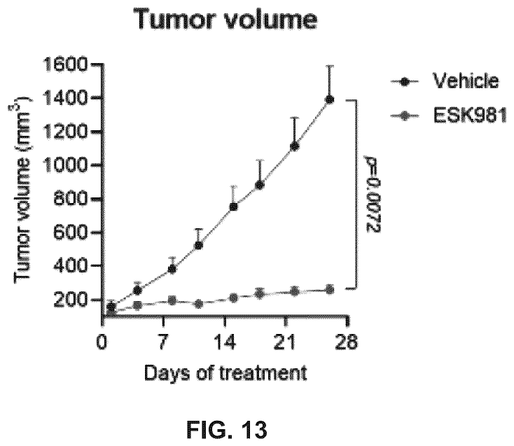

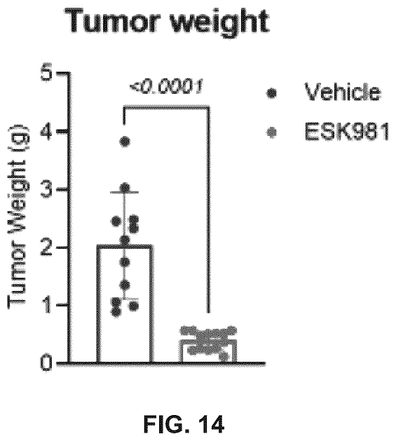

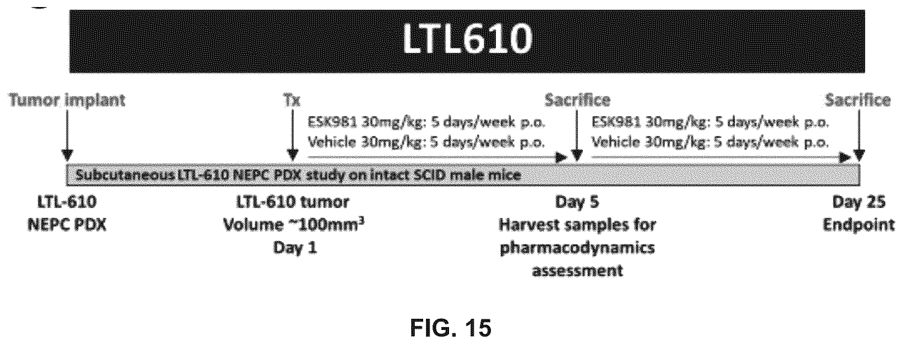

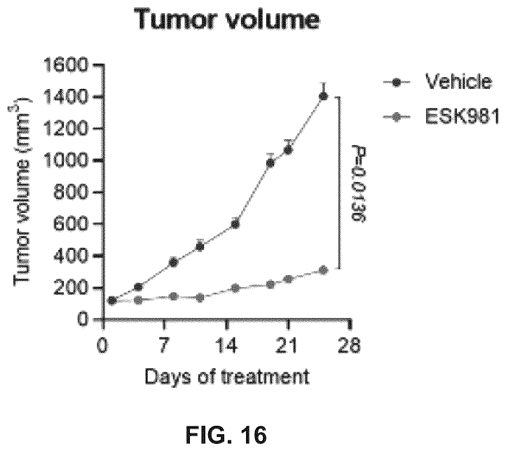

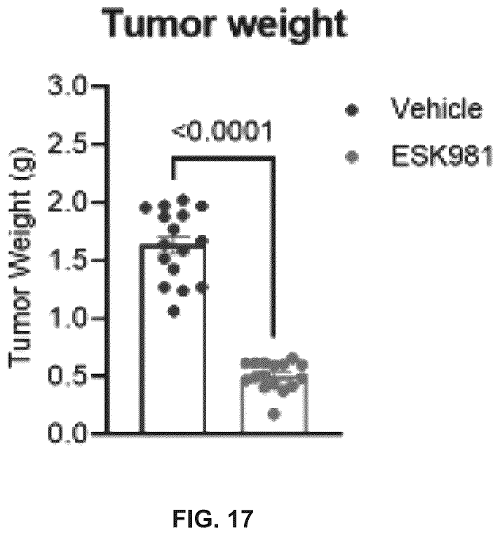

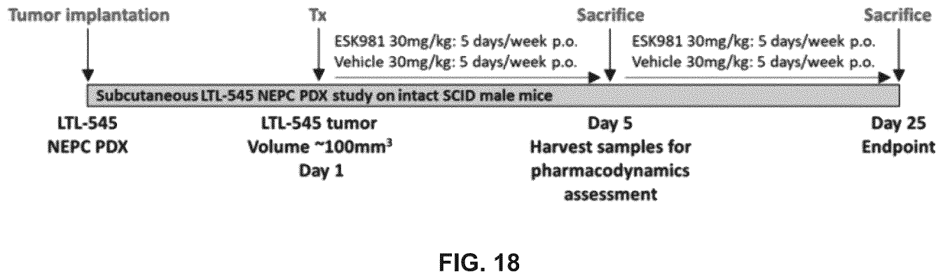

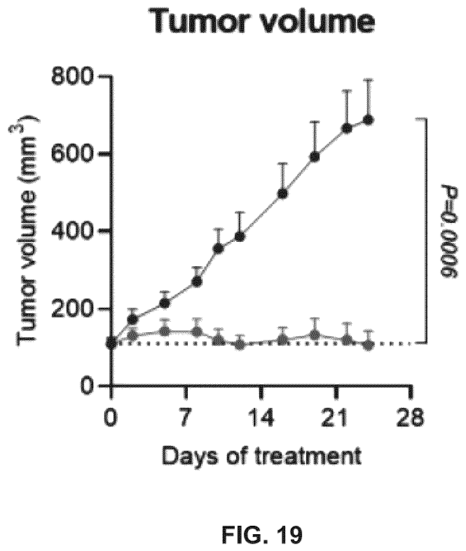

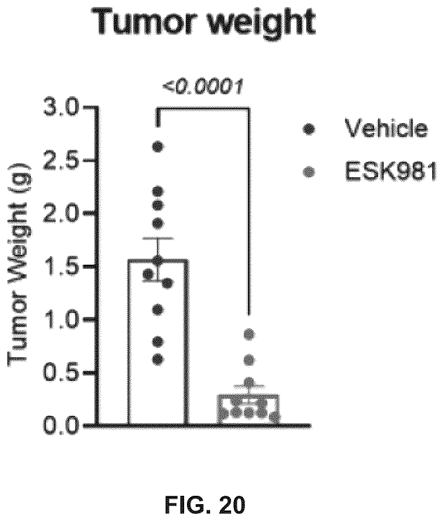

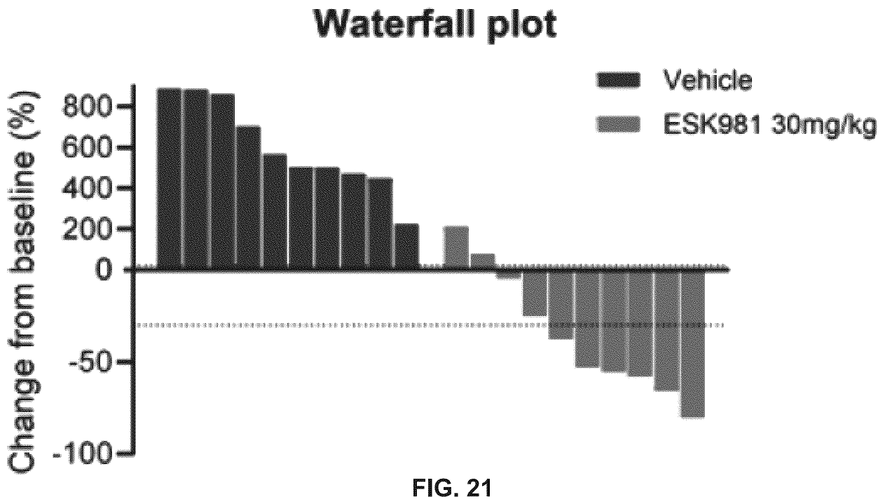

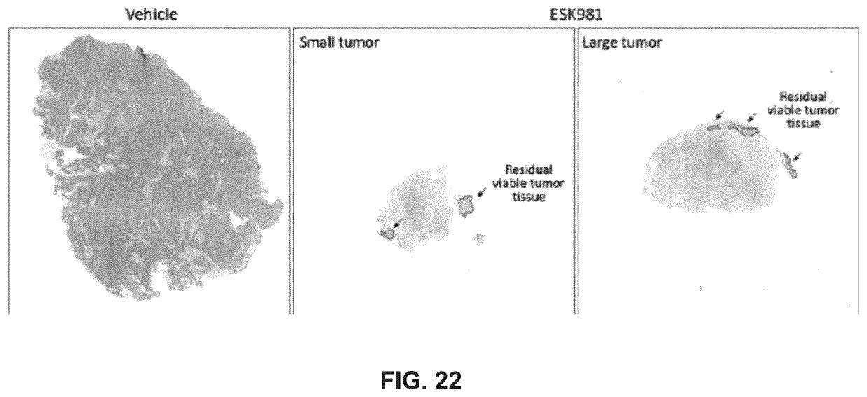

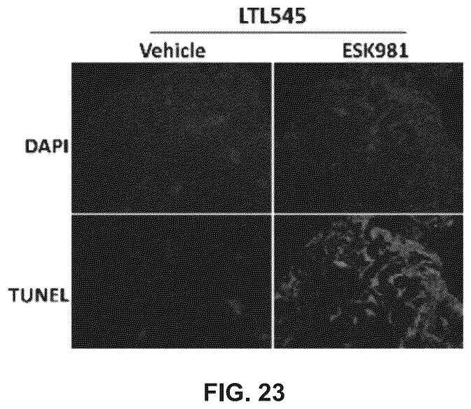

Given the hypoxic and nutrient-depleted environments of NEPC Guo et al., Nat Commun. 2019; 10(1):278; Qi et al., Cancer Cell. 2010; 18(1):23-38., it was hypothesized that NEPC may be dependent on autophagy and sensitive to PIKfyve inhibition by ESK981. To test this NEPC prostate patient-derived xenograft (PDX) of NCI-H660, LTL331R, and LTL610 were subcutaneously grown into non-castrated SCID mice until tumors reached an average size of 100 mm³, after which mice were randomized into two groups. Mice in each group received either vehicle or 30 mg/kg ESK981 five days per week. ESK981 treatment was shown to induce strong tumor inhibitory effects in NCI-H660 NEPC cell line-derived xenografts, while no adverse events were observed with 46 days of treatment (FIGS. 9-11). The anti-tumor effect of ESK981 was in turn confirmed in two additional NEPC PDX models including LTL331R (FIGS. 12-14) and LTL610 xenografts (FIG. 15-17). In these three models, ESK981 showed surprising anti-proliferative effects against NEPC tumors with uniformed responses on individual tumor volume, weight, and gross appearance. Notably, ESK981 cured 80% of tumors in the LTL545 NEPC PDX model (FIGS. 18-23). The average LTL545 tumor volume showed strong tumor regression post-ESK981 treatment with the waterfall plot indicating that 80% of individual LTL545 tumors regressed (FIG. 21). A histopathology evaluation was performed to assess tumor content on dissected responder tumors at endpoint. Of note, there were two non-responding tumors remaining on one mouse. These results mirrored possible patient clinical outcomes and reiterated the importance of using PDXs in preclinical studies. Remarkably, the majority of tumor tissues in the responder tumors were necrotic with only microscopic residual tumor cells regardless of tumor size, suggesting ESK981 may cure NEPC tumors in mice (FIG. 22). ESK981 induced massive cell death in LTL545 NEPC PDX tumors as assessed by TUNEL assays (FIG. 23).

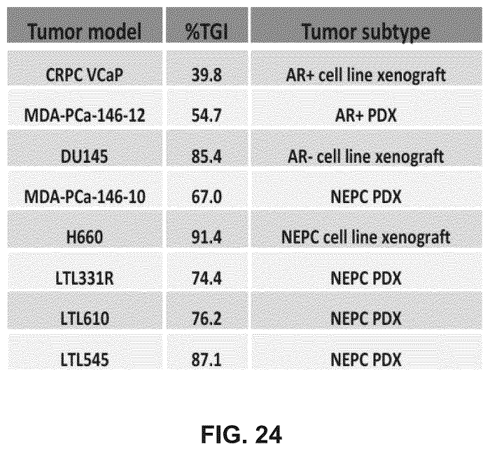

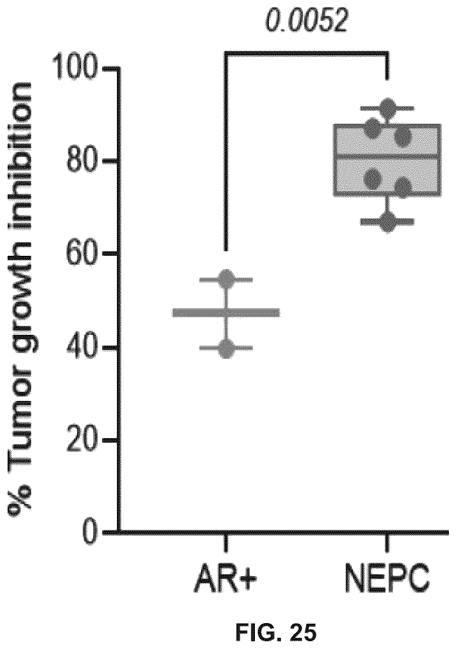

The PIKfyve dependency in several rigorous preclinical in vivo studies with ESK981 treatment in NEPC and AR-positive adenocarcinoma CRPC models has been studied. Percent tumor growth inhibition (TGI %=[1−(RTV of the treated group)/(RTV of the control group)]×100(%)) for each model was calculated and compared between NEPC and AR-positive CRPC tumor models (FIG. 24). Results demonstrated that all six NEPC tumors had higher response rates to ESK981 than AR-positive tumors (FIG. 25). The data suggests that the dual autophagy and angiogenesis inhibitor ESK981 as an effective monotherapy in preclinical NEPC models.

### Example 2

### ESK981 Inhibits Pancreatic Ductal Adenocarcinoma (PDAC) Cancer Growth In Vivo and In Vitro and Induces Unique Vacuolization Morphology

Pancreatic cancer is another fatal malignancy, accounting for approximately 8% of cancer-related deaths in American men and women Siegel et al., CA: A Cancer Journal for Clinicians. 2022; 72(1):7-33. Extensive genomic studies have helped to uncover the molecular underpinnings of pancreatic ductal adenocarcinoma (PDAC) development. Only a minority of cases can be attributed to pathogenic germline variants (up to 10% of patients), with mutations most often occurring in DNA damage repair genes Mizrahi et al., Lancet. 2020; 395(10242):2008-20; Park et al., JAMA. 2021; 326(9):851-62; Hu et al., JAMA. 2018; 319(23):2401-9; Shindo et al., Journal of Clinical Oncology. 2017; 35(30):3382-90. KRAS is the most common somatic mutation found in PDAC (90% of patients) and is also mutated in low-grade precancerous pancreatic intraepithelial neoplasia (PanIN) lesions, thus suggesting that the RAS pathway is a main driver of PDAC development Mizrahi et al., Lancet. 2020; 395(10242):2008-20; Park et al., JAMA. 2021; 326(9):851-62; Connor et al., Nat Rev Cancer. 2022; 22(3):131-42; The ICGC/TCGA Pan-Cancer Analysis of Whole Genomes Consortium. Nature. 2020; 578(7793):82-93; Kanda et al., Gastroenterology. 2012; 142(4):730-3.e9. PMCID: PMC3321090. The next most common somatically altered genes in PDAC include TP53 (80%), CDKN2A (60%), and SMAD4 (40%), with these alterations also often observed in high-grade PanINs Mizrahi et al., Lancet. 2020; 395(10242):2008-20; Park et al., JAMA. 2021; 326(9):851-62; Connor et al., Nat Rev Cancer. 2022; 22(3):131-42; The ICGC/TCGA Pan-Cancer Analysis of Whole Genomes Consortium. Nature. 2020; 578(7793):82-93.

Despite this increased understanding of PDAC development and underlying molecular drivers, little progress has been made in increasing survival rates Siegel et al., CA: A Cancer Journal for Clinicians. 2022; 72(1):7-33. The overall five-year survival rate for pancreatic cancer is only 10% Mizrahi et al., Lancet. 2020; 395(10242):2008-20; Park et al., JAMA. 2021; 326(9):851-62; Connor et al., Nat Rev Cancer. 2022; 22(3):131-42. Pancreatic cancer is particularly lethal since early symptoms are rare, and more than 50% of patients have distant metastatic disease at diagnosis, with the most common sites of metastases being the liver and lungs Mizrahi et al., Lancet. 2020; 395(10242):2008-20; Park et al., JAMA. 2021; 326(9):851-62; Connor et al., Nat Rev Cancer. 2022; 22(3):131-42. Only 20% of patients present with localized, resectable tumors Park et al., JAMA. 2021; 326(9):851-62; Connor et al., Nat Rev Cancer. 2022; 22(3):131-42. The stage of the cancer at diagnosis is inversely related to prognosis, with 40% five-year survival rates for localized PDAC and only 2% five-year survival rates for distant metastatic disease Connor et al., Nat Rev Cancer. 2022; 22(3):131-42. For patients with advanced metastatic PDAC, the only treatment options are systemic therapies Mizrahi et al., Lancet. 2020; 395(10242):2008-20; Park et al., JAMA. 2021; 326(9):851-62. The current first-line treatments for metastatic PDAC are gemcitabine plus nab-paclitaxel or FOLFIRINOX (5-fluorouracil, folinic acid, irinotecan, and oxaliplatin), but survival even on these regimens is only a matter of months Conroy et al., N Engl J Med. 2011; 364(19):1817-25; Von et al., N Engl J Med. 2013; 369(18):1691-703. PMCID: PMC4631139. With limited treatment options that are not curative, there remains a critical need for additional therapeutic options for pancreatic cancer patients.

One of the potential therapeutic vulnerabilities of PDAC is its increased dependence on autophagy for survival. Autophagy levels have been shown to be elevated in high-grade PanIN lesions and PDAC compared to normal ductal epithelium and low-grade PanIN lesions Yang et al., Genes Dev. 2011; 25(7):717-29. Growth and progression of certain PDAC models, including those with mutant TP53, have been shown to be dependent on autophagy for survival Yang et al., Genes Dev. 2011; 25(7):717-29; Yang et al., Cancer Discov. 2014; 4(8):905-13. Since mutant KRAS is an important driver of PDAC, immense effort has been spent on developing therapies targeting KRAS or the downstream signaling cascade, MEK/ERK Piffoux et al., Br J Cancer. 2021; 124(2):333-44. PMCID: PMC7852577. Interestingly, MEK/ERK signaling inhibitors increase autophagy levels in PDAC as a resistance mechanism against the cytotoxic effects of the drugs, and dual inhibition of MEK/ERK and autophagy leads to enhanced anti-tumor effects Bryant et al., Nat Med. 2019; 25(4):628-40.; Kinsey et al., Nat Med. 2019; 25(4):620-7. PMCID: PMC6452642. Autophagy has also been shown to play an important role in immune evasion by PDAC as enhanced autophagy leads to decreased antigen presentation via lysosomal degradation of MHC-I in tumor cells Yamamoto et al., Nature. 2020; 581(7806):100-5.

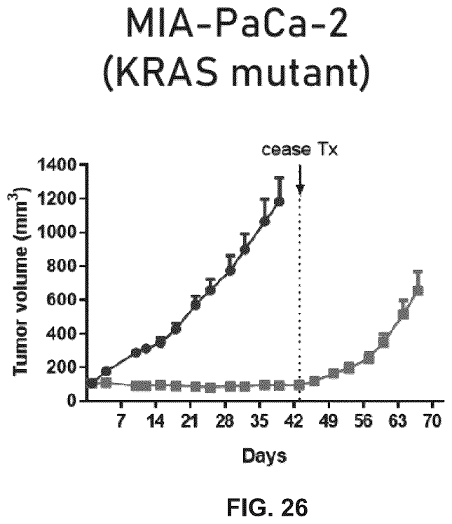

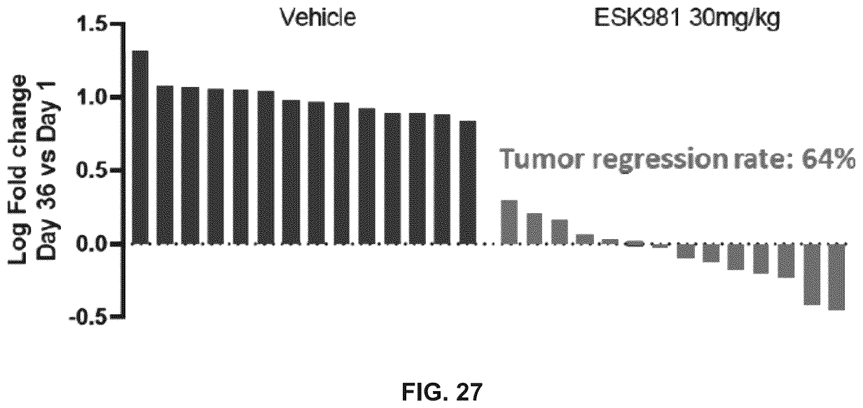

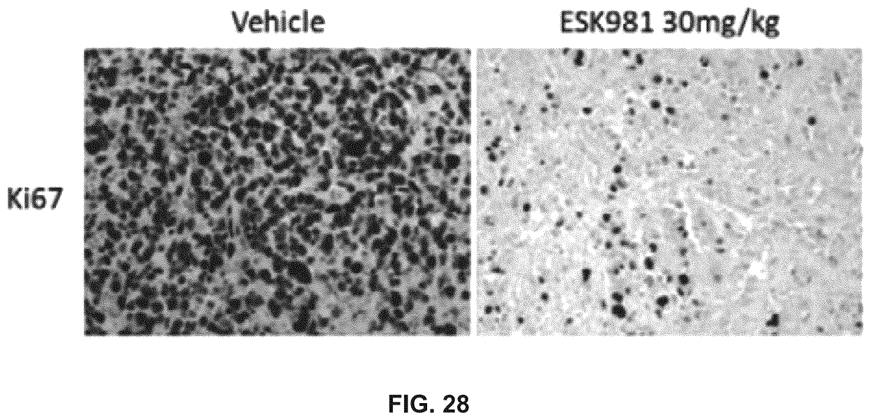

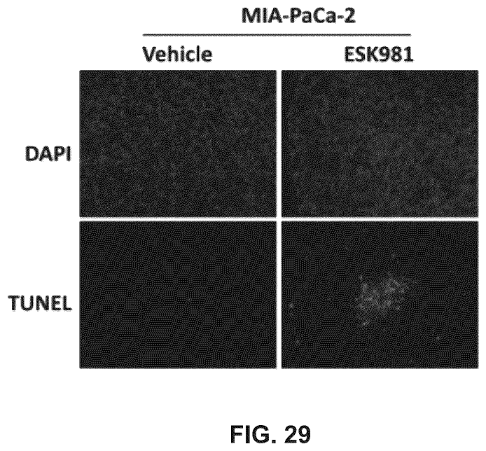

Given the enhanced autophagy dependence of PDAC and the urgent need for new therapies, ESK981 was investigated for efficacy in combatting this type of cancer. Xenograft experiments with the MIA-PaCa-2 human PDAC cell line, which possesses a KRAS mutation, in non-castrated SCID mice. Tumors were grown until they reached an average size of 100 mm³, after which mice were randomized into two groups that were treated with vehicle or 30 mg/kg ESK981 five days a week. The tumor growth curve showed remarkable prevention of tumor growth in mice treated with ESK981 (FIG. 26). Upon stopping ESK981 treatment, tumors began to proliferate again, demonstrating the key role of autophagy in sustaining tumor growth in this cell line (FIG. 26). Waterfall plots at Day 36 versus Day 1 showed that there was a high rate of tumor regression (64%) in mice treated with ESK981 (FIG. 27). Tumors from ESK981-treated mice had drastic reductions in Ki67 staining (FIG. 28) and increases in cell death measured by TUNEL assays (FIG. 29).

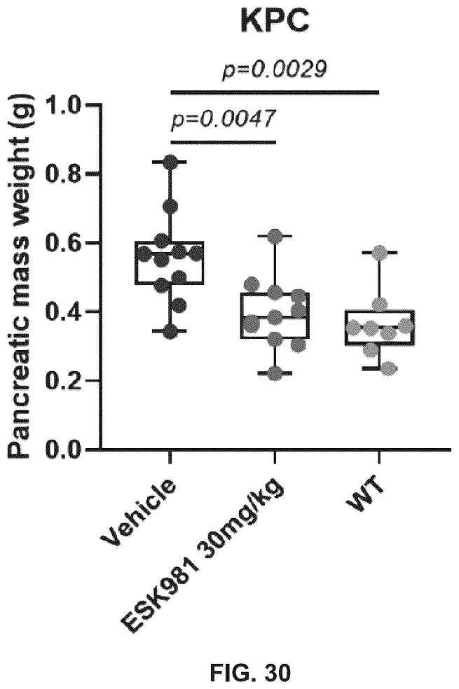

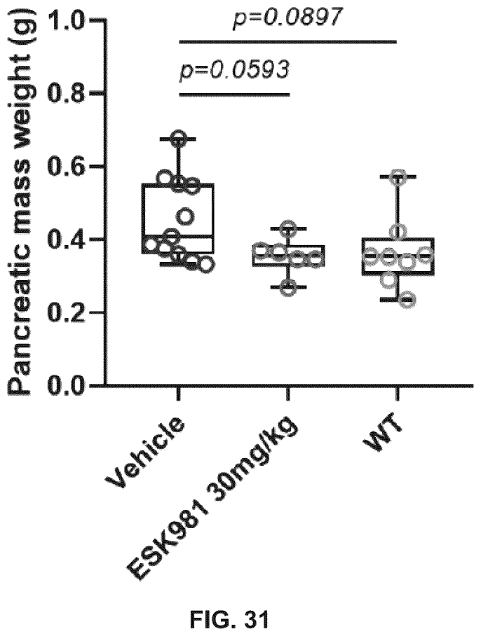

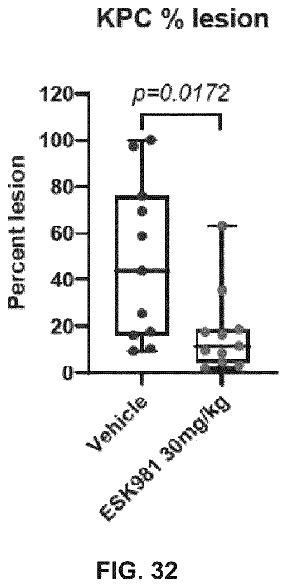

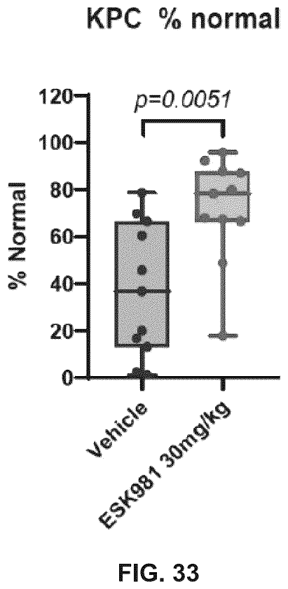

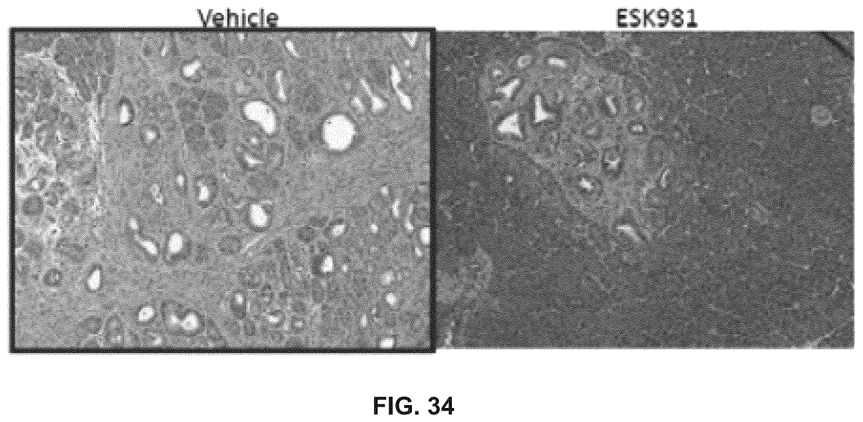

ESK981 was also found to be a potent anti-cancer agent in KRAS-driven genetically engineered mouse models (GEMMs) of PDAC. We analyzed pancreatic mass weight in two different GEMMs—the KC model (Kras^{G12D/+}; p48Cre) and KPC model (Kras^{G12D/+}; Trp53^{R172H/+}; p48Cre). In both murine models, treatment with 30 mg/kg ESK981 reduced pancreatic mass weight to that of wild-type mice, with greater significance achieved in the KPC model containing both Kras and Trp53 mutations (FIGS. 30 and 31). Pathologic assessment of KPC mice showed that ESK981-treated mice had decreased numbers of lesions and increased percentages of normal pathology (FIGS. 32-34).

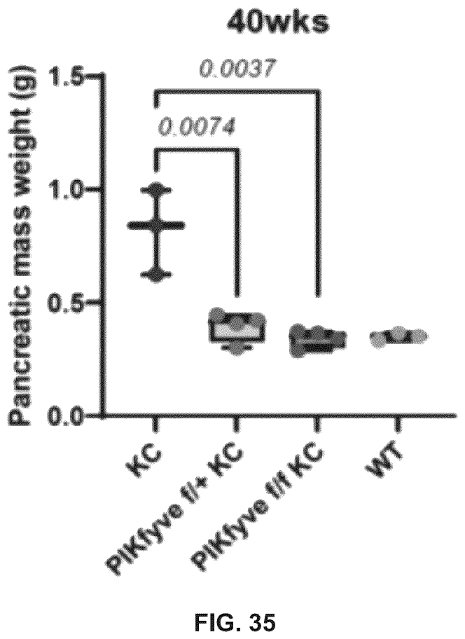

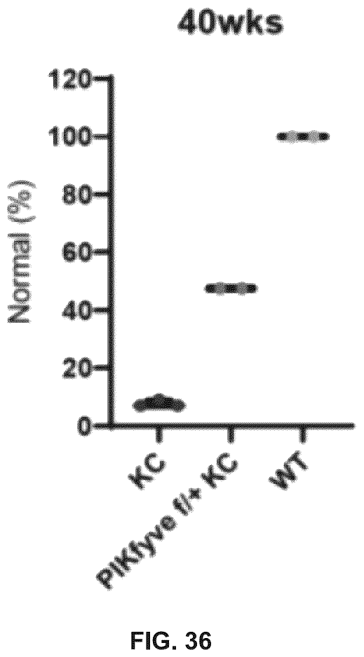

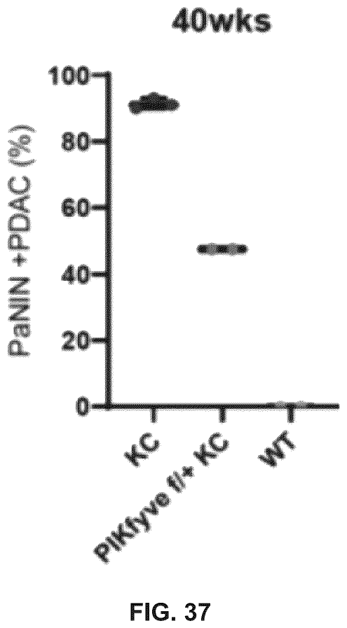

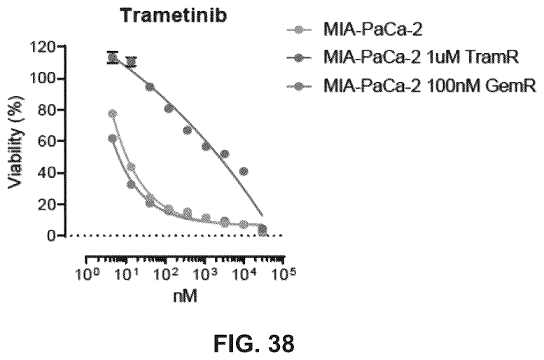

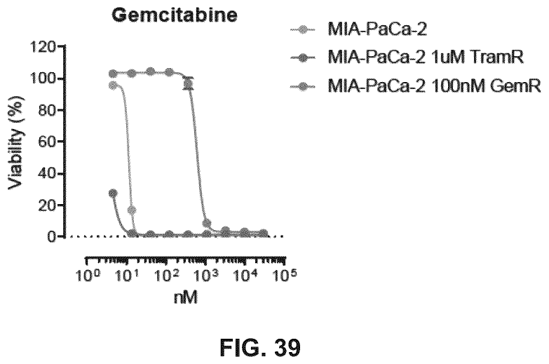

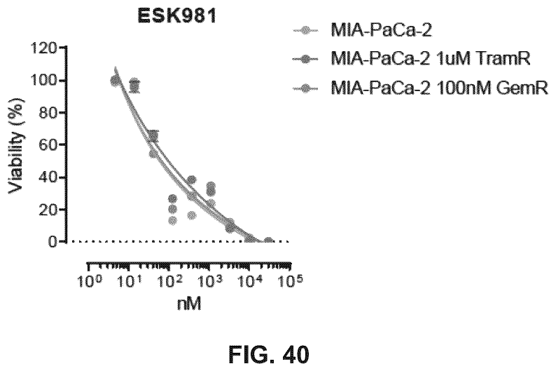

Pikfyve was next genetically inactivated in the KC model to assess whether PIKfyve inhibition could recapitulate the findings with ESK981. Indeed, pancreatic mass weights decreased in KC mice bearing deletion of one or two alleles of Pikfyve, reducing pancreatic weights to those found in wild-type mice (FIG. 35). Heterozygous deletion of Pikfyve led to increased normal pathology (FIG. 36) and decreased PanIN and PDAC lesions compared to KC mice (FIG. 37). Together, these data show that the PIKfyve inhibitor, ESK981, has potent anti-tumor effects in both xenograft models and GEMMs of PDAC. Importantly, ESK981 testing in trametinib- and gemcitabine-resistant PDAC cell lines, generated through in vitro culturing, was able to inhibit cell growth at similar potencies across cell lines (FIGS. 38 and 40).

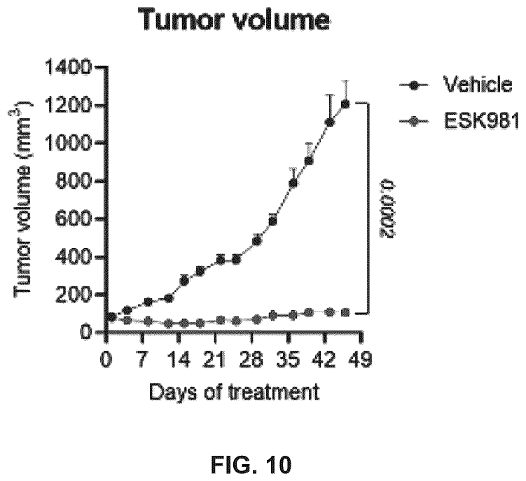

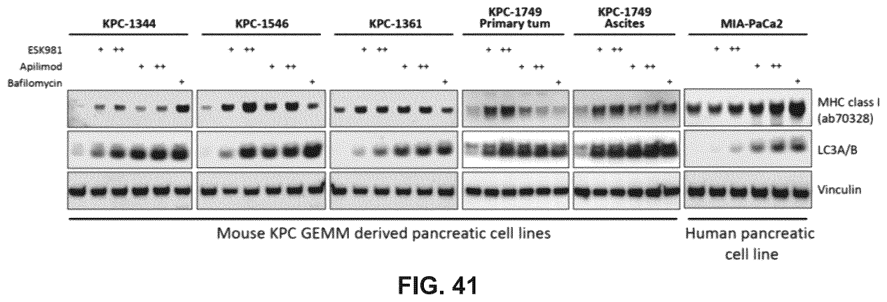

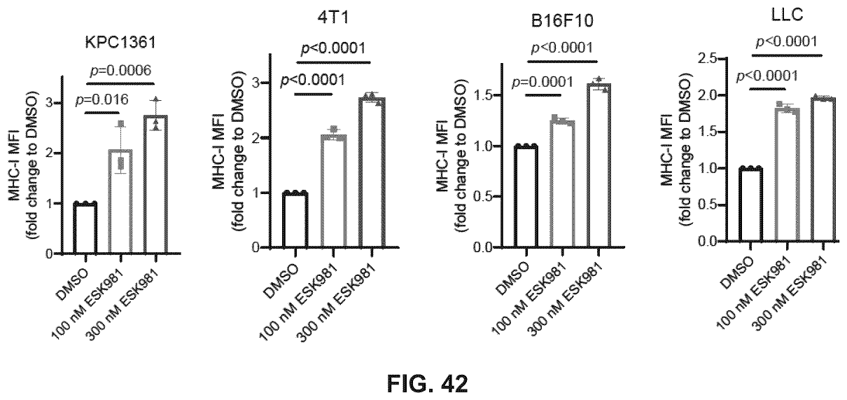

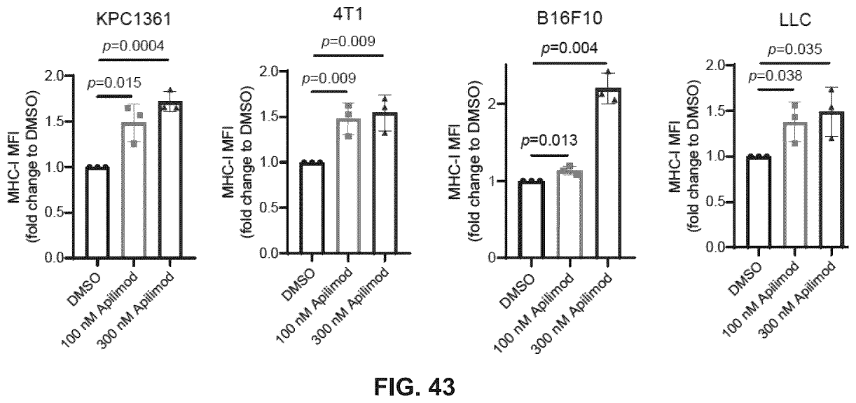

Autophagy can play an important role in maintenance of cancer cell survival through immunomodulatory mechanisms. Specifically, autophagy in PDAC promotes lysosomal degradation of MHC class I molecules. MHC-I levels are enriched inside lysosomes and autophagosomes of PDAC cells and depleted on the cell surfaces, leading to decreased antigen presentation and immune evasion Yamamoto et al., Nature. 2020; 581(7806):100-5. PMCID: PMC7296553. It was evaluated whether autophagy inhibition with ESK981 affected MHC-I levels in mouse and human PDAC cell lines. As shown in FIG. 10, ESK981 increased levels of MHC-I in multiple cell lines derived from KPC mice, as well as in MIA-PaCa-2 cells. Apilimod (another PIKfyve inhibitor) or bafilomycin (autophagy inhibitor) showed similar results (FIG. 41). Flow cytometry validated that ESK981 (FIG. 42) and apilimod (FIG. 43) increased cell surface expression of MHC-I in pancreatic cancer cells, and these findings were extended across other cell lineages (breast cancer (4T1), melanoma (B16-F10), and lung cancer (LLC)).

### Example 3

### ESK981 Inhibits Pancreatic Neuroendocrine Tumors (PNETs) Cancer Growth In Vitro and In Vivo

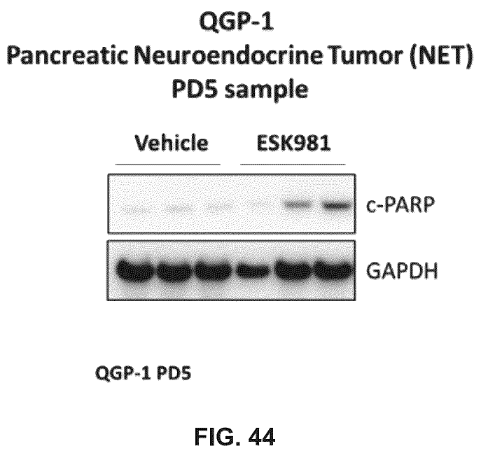

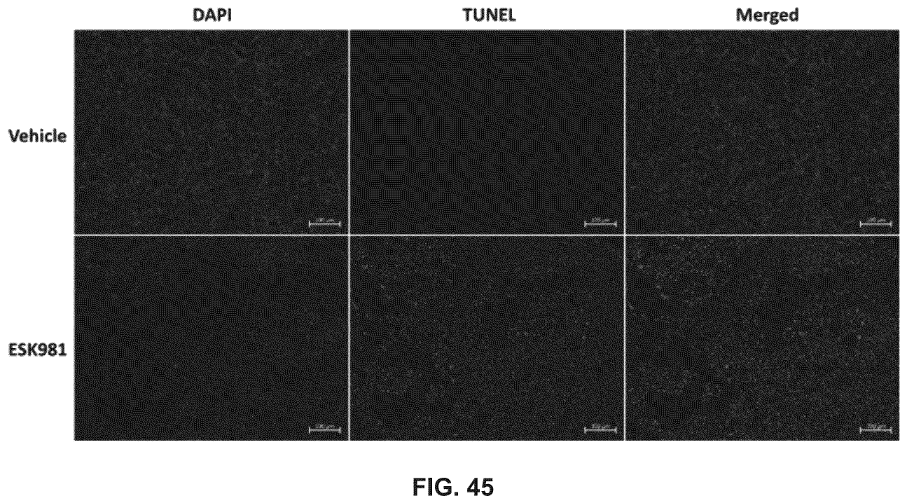

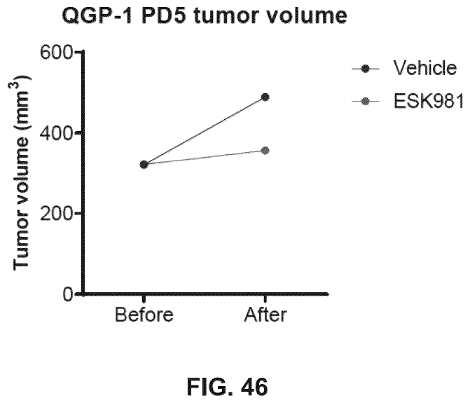

Data shown above found that NEPC was particularly sensitive to autophagy inhibition. In addition to NEPC, autophagy plays an important role in neuroendocrine lung tumor proliferation and survival Hong et al., Int J Oncol. 2013; 43(6):2031-8. Combined, these results suggest that neuroendocrine tumors may share a common feature of autophagy-dependence and be more sensitive to autophagy inhibition than non-neuroendocrine tumors. A small subset (1-2%) of all pancreatic cancers are classified as pancreatic neuroendocrine tumors (PNETs), a heterogenous group of tumors that has an overall better prognosis than PDAC but still has the potential to metastasize Chang et al., Annual Review of Medicine. 2022; 73(1):213-29; Maharjan et al., Cancers. 2021; 13(20):5117. Given the efficacy of ESK981 in NEPC, the activity of ESK981 in xenografts with the human PNET cell line, QGP-1, was analyzed. Mice bearing PDX QGP-1 tumors were treated with vehicle or 30 mg/kg ESK981 for five days and sacrificed for analysis. ESK981 treatment increased apoptosis in tumors, as evidenced by both increased c-PARP levels (FIG. 44) and TUNEL staining of tumors (FIG. 45). Although only a five-day experiment, tumor volume also trended downward in the ESK981-treated mice (FIG. 46). These data suggest that, in addition to PDAC, ESK981 may be effective in suppressing tumor growth in cases of PNET due to enhanced cellular apoptosis.

Combined, these results identify prostate and pancreatic cancers to be autophagy-dependent malignancies sensitive to autophagy inhibitors, particularly neuroendocrine neoplasms of both lineages.

### Example 4

### Pikfyve is Dispensable for Normal Pancreas but is Required for PDAC Development

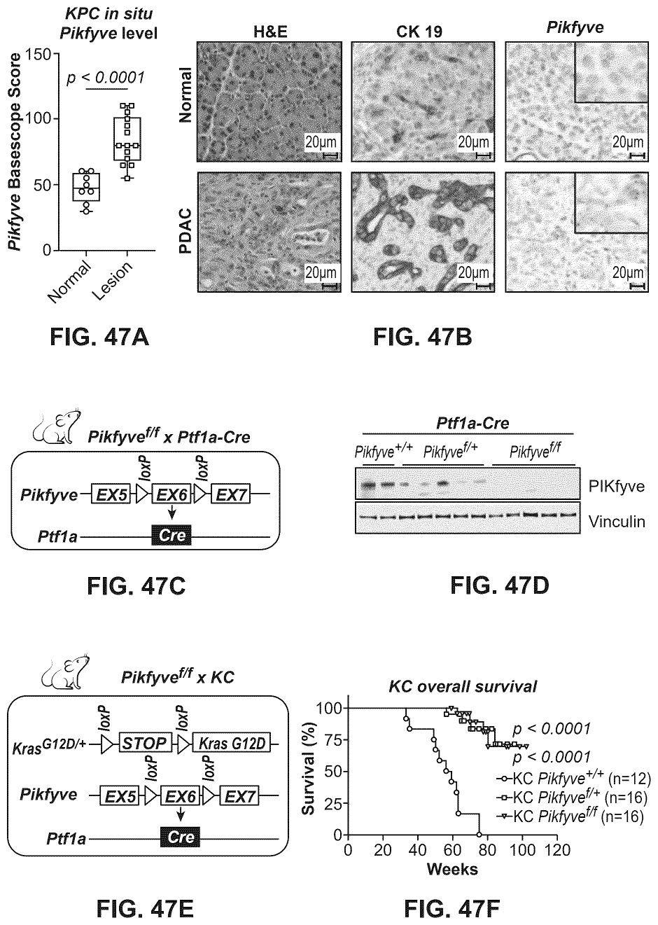

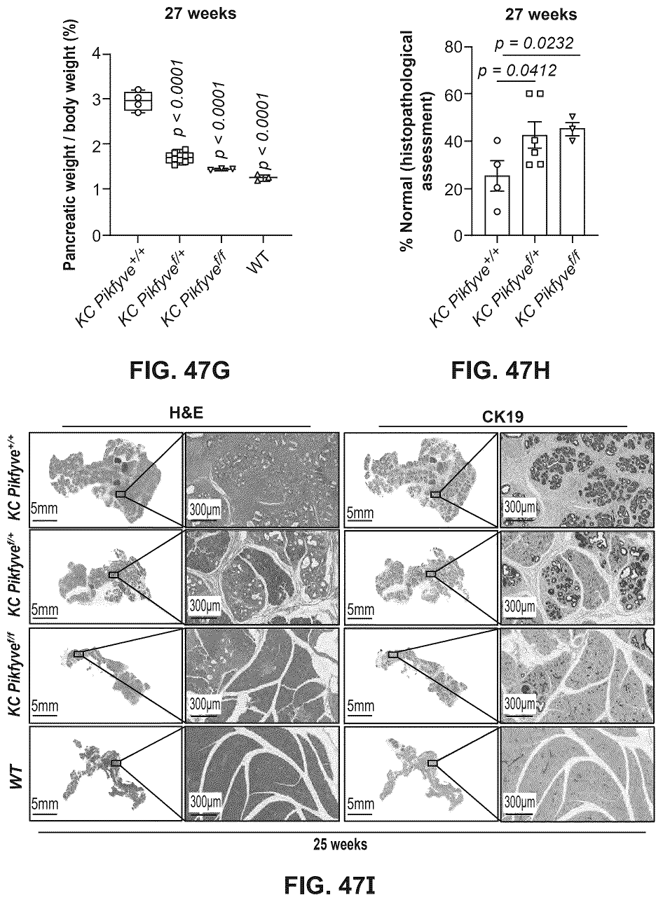

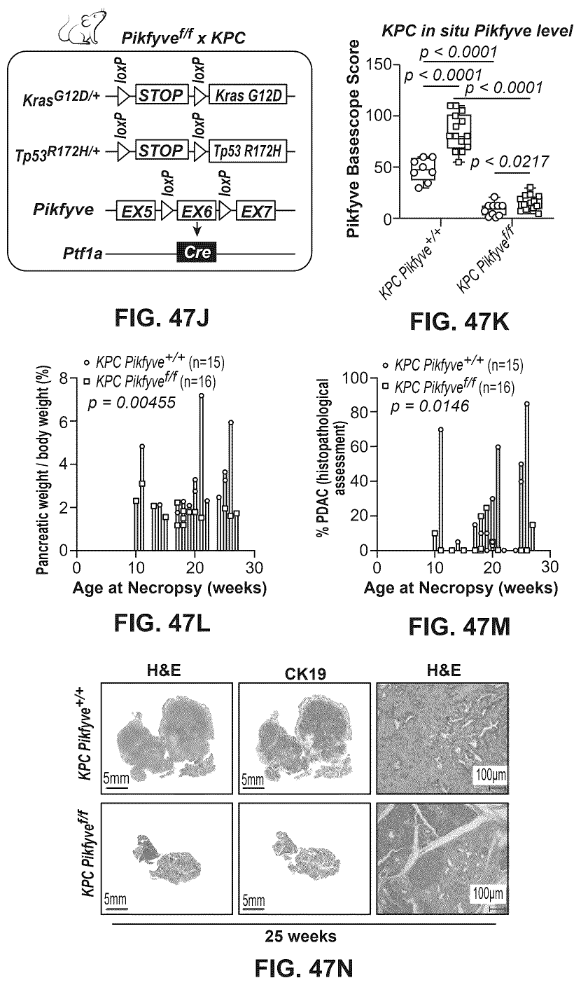

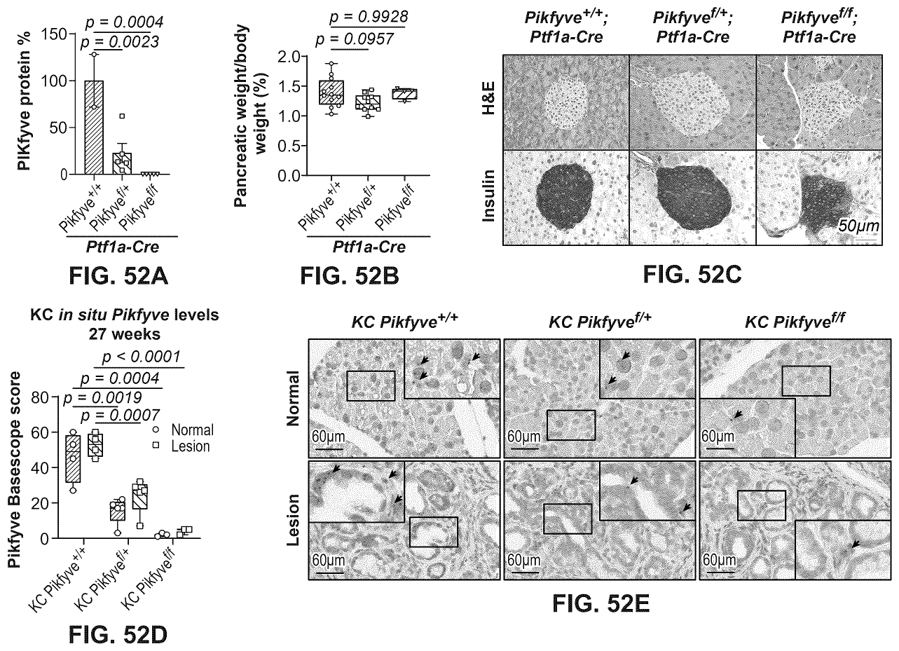

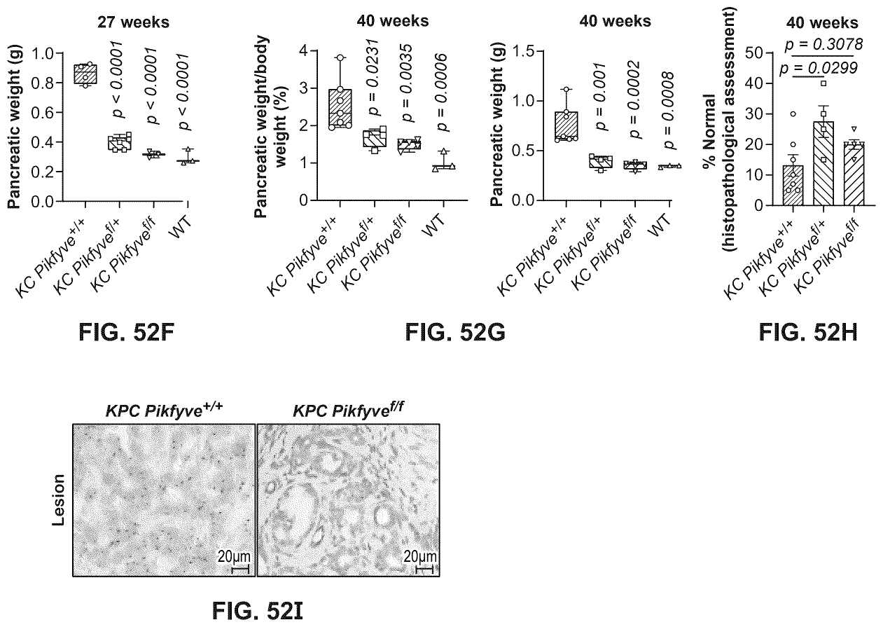

To study the role of Pikfyve in pancreatic cancer development, Pikfyve expression was evaluated in the autochthonous PDAC GEMM Pft1a-Cre; LSL-Kras^{G12D/+}; LSL-Trp53^{R172H/+} (KPC). Using RNA-ISH with a probe targeting Pikfyve exon 6, it was found that Pikfyve expression was dramatically and consistently higher in lesion tissue compared to surrounding normal tissue in situ (FIG. 47A,B). These data suggest that PDAC may have an elevated utilization of PIKfyve-driven processes, relative to normal pancreatic tissue. To assess this, conditional pancreatic Pikfyve knockout mice were generated using the Ptf1a promoter-driven Cre recombinase (Ptf1a-Cre; Pikfyve^{f/f}) (FIG. 47C). Upon confirming the loss of PIKfyve protein in pancreatic tissue (FIG. 47D, FIG. 52A), the physiological impact of Pikfyve loss on pancreatic development was assessed. It was observed that Pikfyve loss did not impact pancreatic weight, morphology, or function in terms of insulin production (FIG. 52B,C, suggesting that Pikfyve is not critical for normal pancreatic tissue development or function.

The effect of Pikfyve loss on PDAC development by crossing Pikfyve^{+/+}, Pikfyve^{f/+}, or Pikfyve^{f/f} with the KC model (Ptf1a-Cre; LSL-Kras^{G12D/+}), was then evaluated to assess pancreatic tumorigenesis (FIG. 47E). First a decrease in Pikfyve transcript in the pancreata of KC Pikfyve^{f/+} and KC Pikfyve^{f/f} mice was confirmed (FIG. 52D,E). In monitoring these cohorts of mice, it was found that Pikfyve loss substantially extended the survival of mice harboring the KC genotype (FIG. 47F). To determine whether this was correlated with a difference in pancreatic disease burden, the pancreata of a separate cohort of mice was evaluated. It was shown that compared to pancreata of KC Pikfyve^{+/+} littermates, pancreata of KC Pikfyve^{f/+} and KC Pikfyve^{f/f} mice weighed less and were closer in weight to pancreata of wild-type mice at 27 weeks of age (FIG. 47G, FIG. 52F). Additionally, pancreata of KC mice with Pikfyve loss retained a higher degree of normal histological structures based on hematoxylin and eosin (H&E) staining or immunohistochemistry (IHC) staining for cytokeratin 19 (CK19) (FIG. 47H,I). Consistent results were recapitulated at a later age of 40 weeks, as well as both on macroscopic and microscopic evaluations (FIG. 52G,H).

Next the role of Pikfyve in the KPC model was evaluated to assess the impact of PIKfyve on tumor progression (FIG. 47J,K, FIG. 52I). Given that KPC mice are known to suffer from disease independent of PDAC, the impact of Pikfyve loss on PDAC development was assessed by measuring the pancreatic weight of mice in KPC Pikfyve^{+/+} or KPC Pikfyve^{f/f} cohorts upon death, agnostic of cause. Yang, A. et al., Cancer Discov. 2014:4: 905-913. At all stages of experimental assessment, KPC Pikfyve^{f/f} pancreata weighed less than those of KPC Pikfyve^{+/+} adjusting for age (FIG. 47L). In accordance with this, the pancreata from KPC Pikfyve^{+/+} mice displayed significantly and consistently lower degrees of PDAC burden based on histological evaluations employing H&E and IHC staining for CK19 (FIG. 47M,N). Taken together, these data indicate that Pikfyve loss suppresses pancreatic cancer onset and progression in the KC and KPC models, respectively, while not affecting normal pancreatic tissue. Collectively, these studies with GEMMs suggest that PDAC has an elevated requirement for PIKfyve-driven processes.

### Example 5

### Pharmacological Inhibition of PIKfyve Suppresses PDAC Development and Growth

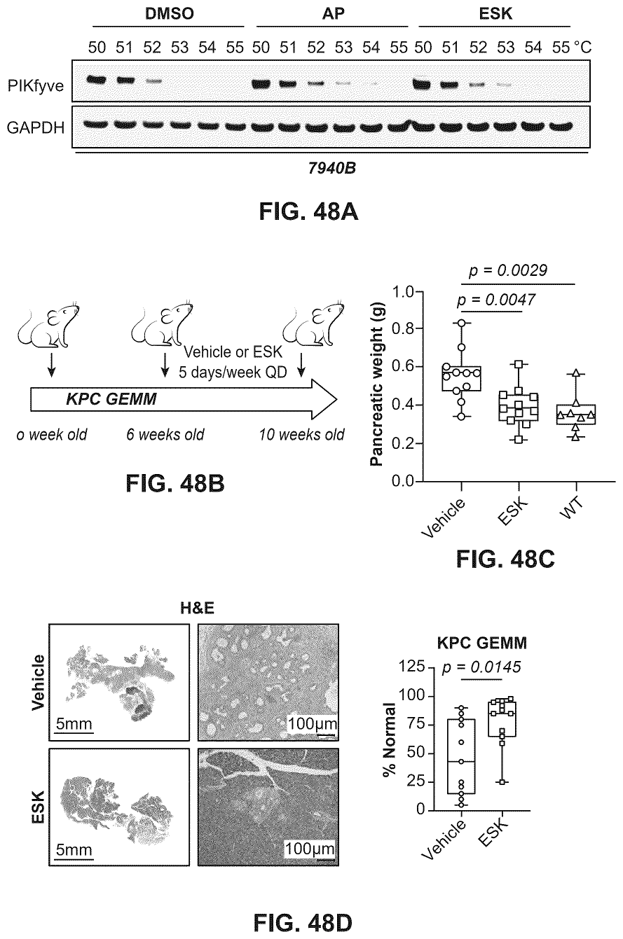

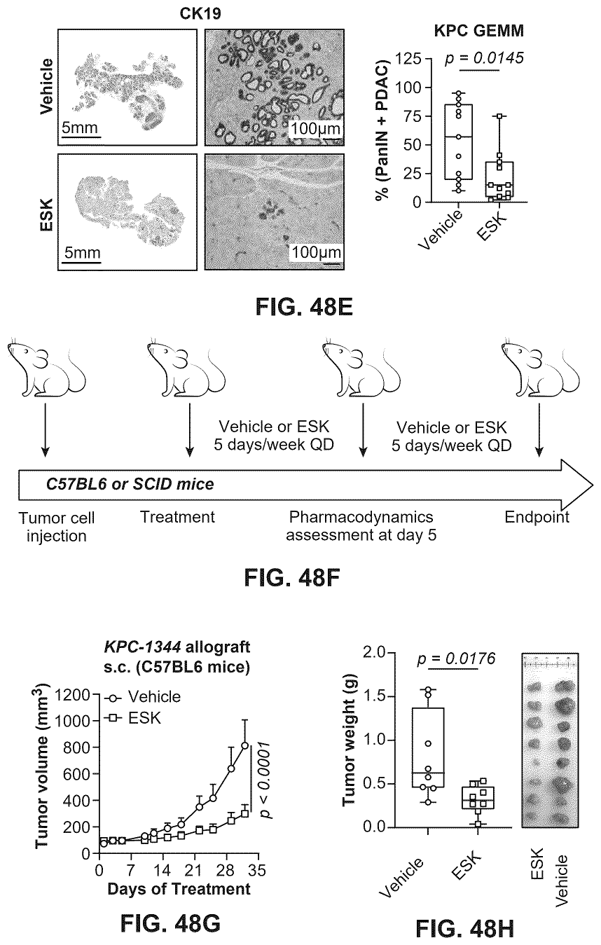

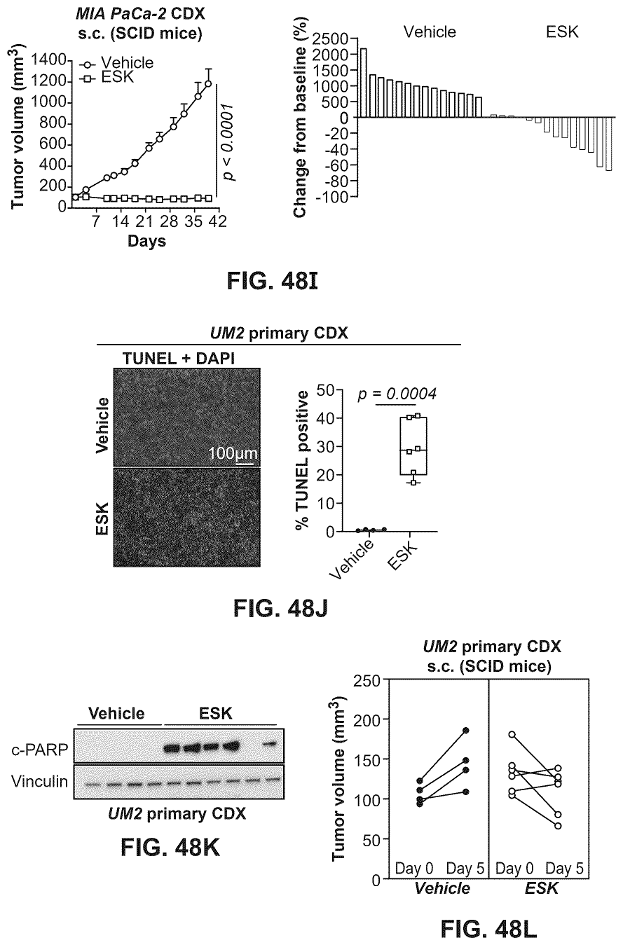

Given that genetic perturbation of Pikfyve attenuated PDAC development, whether pharmacological PIKfyve inhibition would elicit similar effects was next evaluated. First apilimod and ESK981, two PIKfyve inhibitors that have cleared phase 1 clinical trials were confirmed to bind to mouse PIKfyve protein using Cellular Thermal Shift Assay (CETSA) (FIG. 48A). Pili, R., et al., Invest New Drugs 2014:32: 1258-1268; Harb, W. A. et al., Blood 2017:130: 4119-4119. Given that apilimod is known to have poor in vivo pharmacokinetics, ESK981 was focused on for subsequent in vivo experiments. Ikonomov, O. C., et al., Toxicol. Appl. Pharmacol. 2019:383: 114771. To evaluate the impact of PIKfyve inhibition on PDAC development, a cohort of KPC mice aged to 6 weeks, was prophylactically treated with ESK981 for 4 weeks (FIG. 48B). At the endpoint of 10 weeks, it was observed that the weights of KPC pancreata treated with ESK981 treatment were reduced to levels approaching that of wild-type pancreata (FIG. 48C). On histopathological evaluation, ESK981 therapy-treated mice exhibited an increased retention of histopathologically unremarkable pancreatic tissue, including both acinar and endocrine components in normal physiological proportion, and relatively reduced PanIN or PDAC burden. These conclusions were based on exhaustive morphological evaluation by H&E, which were then broadly cross-validated by CK19 IHC staining (FIG. 48D,E).

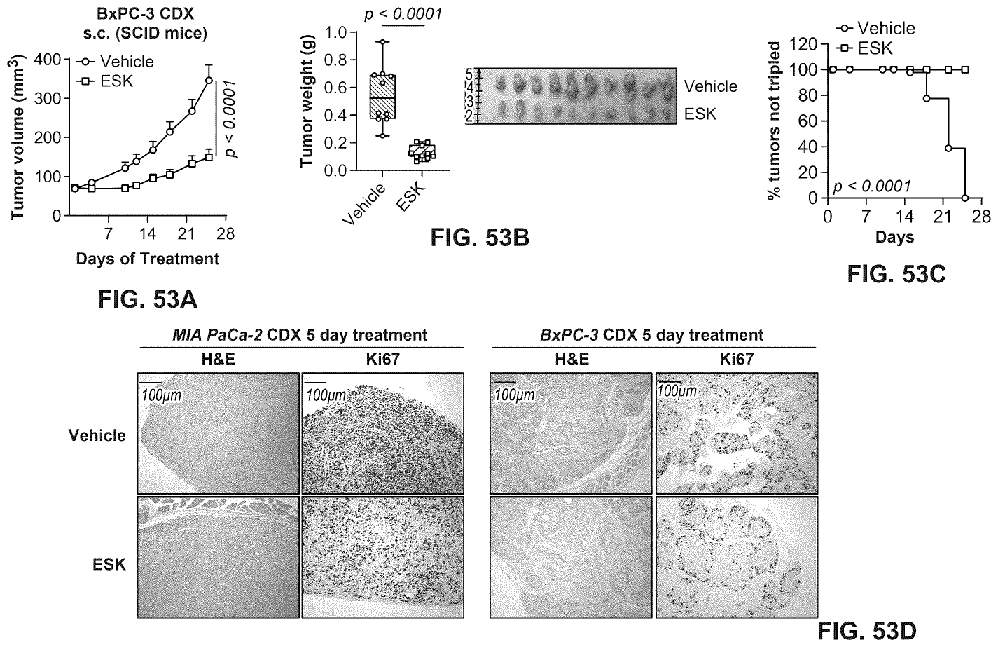

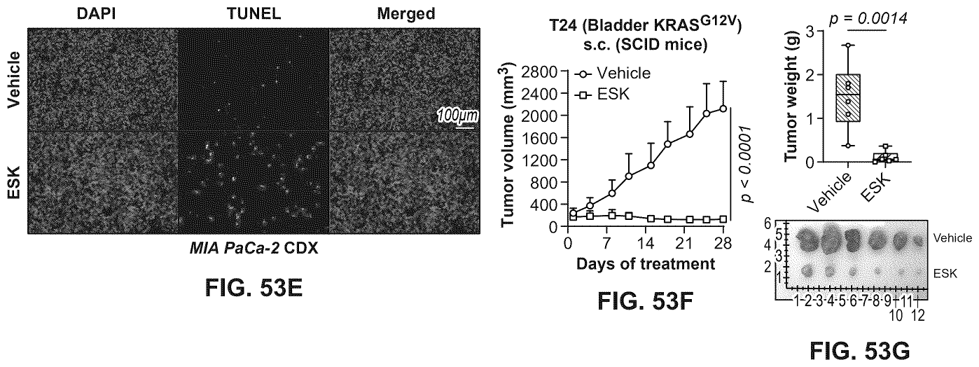

Next, to determine the impact of PIKfyve inhibition on PDAC tumor growth, an in vivo allograft and xenograft models, were employed to test the efficacy of ESK981 (FIG. 48F). In a KPC derived subcutaneous syngeneic allograft, ESK981 therapy reduced tumor growth and weight at the endpoint (FIG. 48G,H). Similarly, ESK981 completely suppressed the growth of MIA PaCa-2 cell-derived xenograft (CDX) tumors (FIG. 48I). To assess the impact of PIKfyve inhibition on non-KRAS driven PDAC, a BxPC-3 CDX was employed. This study showed that ESK981 suppressed tumor growth and reduced tumor weight at the studies endpoint (FIG. 53A-C). In both the MIA PaCa-2 and BxPC-3 CDX models, ESK981 treatment reduced the proliferation of these tumors based on Ki-67 staining (FIG. 53D). Further, ESK981 treatment induced substantial apoptosis in the MIA PaCa-2 as well as a UM2 primary CDX model, as shown by increased Terminal dUTP Nick End Labeling (TUNEL) staining and PARP cleavage (FIG. 48J,K, FIG. 53E) after 5 days of treatment. Regression in most of the tumors in the UM-2 primary CDX cohort upon ESK981 treatment was also observed (FIG. 48L). Finally, it was found that ESK981 treatment elicited similar effects on a KRAS^{G12V}-driven T24 bladder CDX (FIG. 53E-G). Taken together, these data show that PIKfyve inhibition decreases proliferation, induces apoptosis, and dramatically suppresses growth in both murine and human PDAC tumor models.

### Example 6

### PIKfyve Perturbation Suppresses Autophagy and Decreases PDAC Cell Proliferation

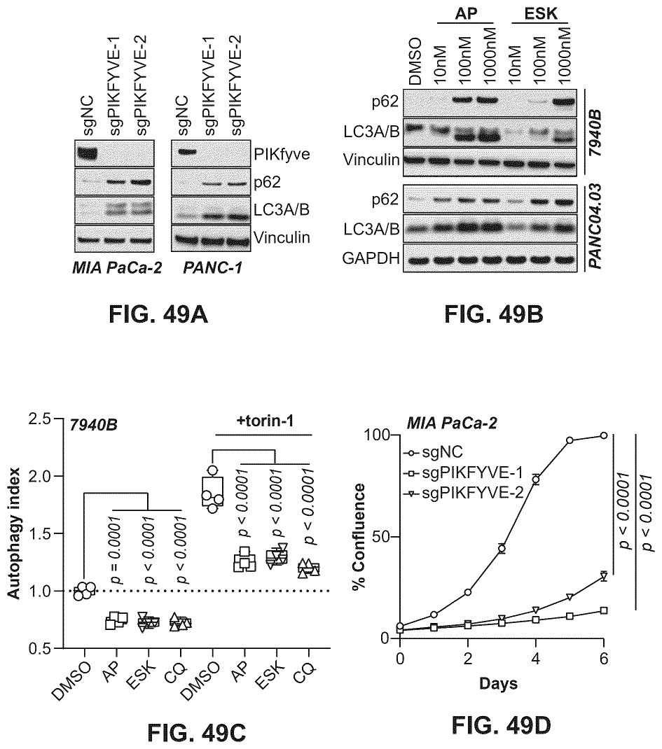

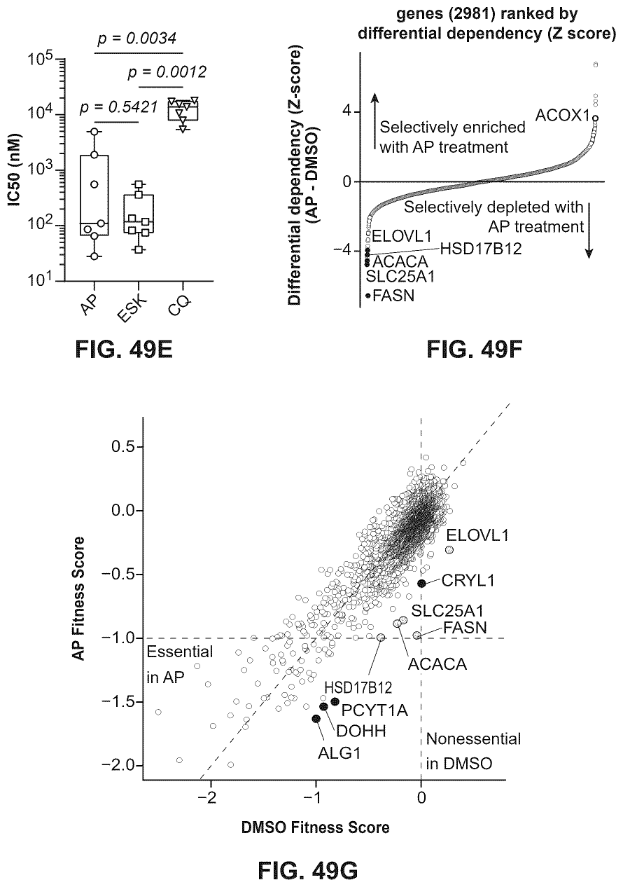

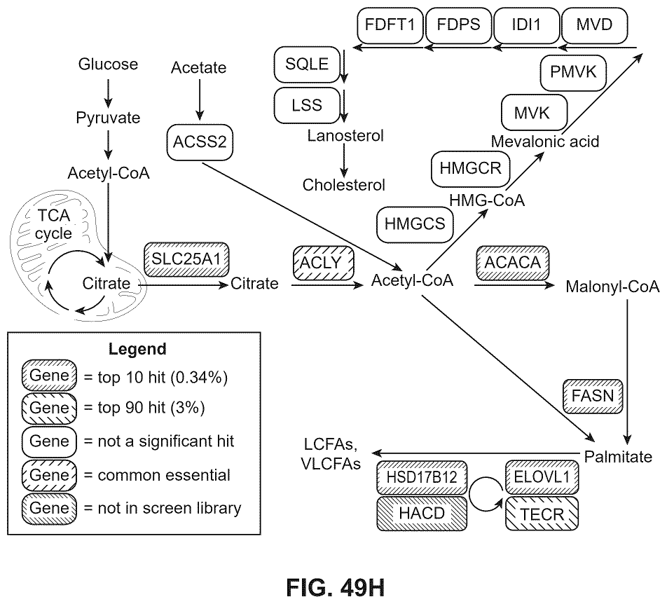

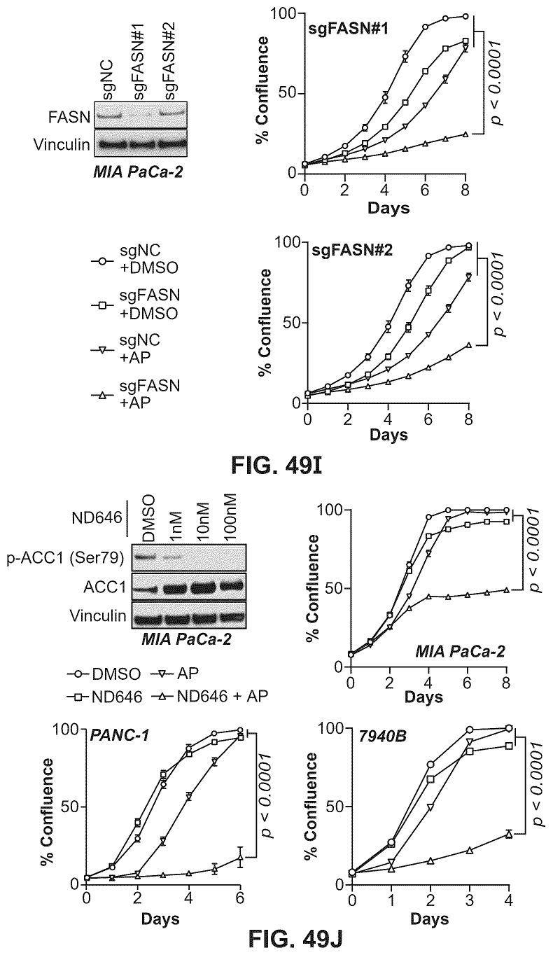

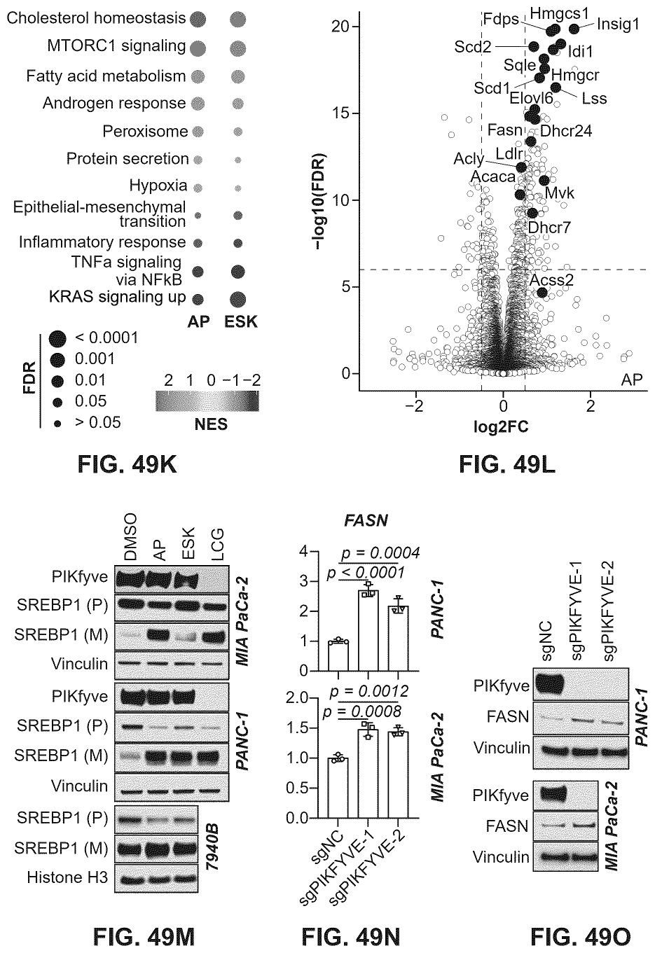

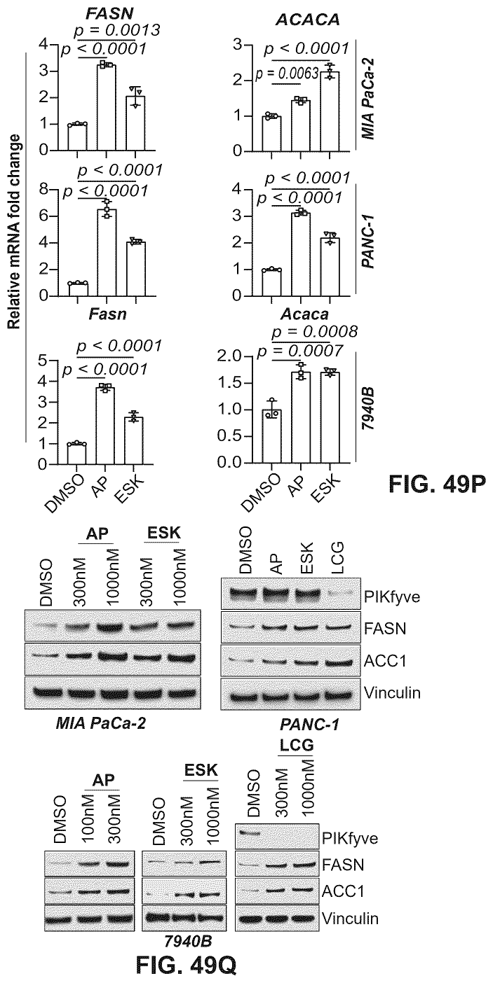

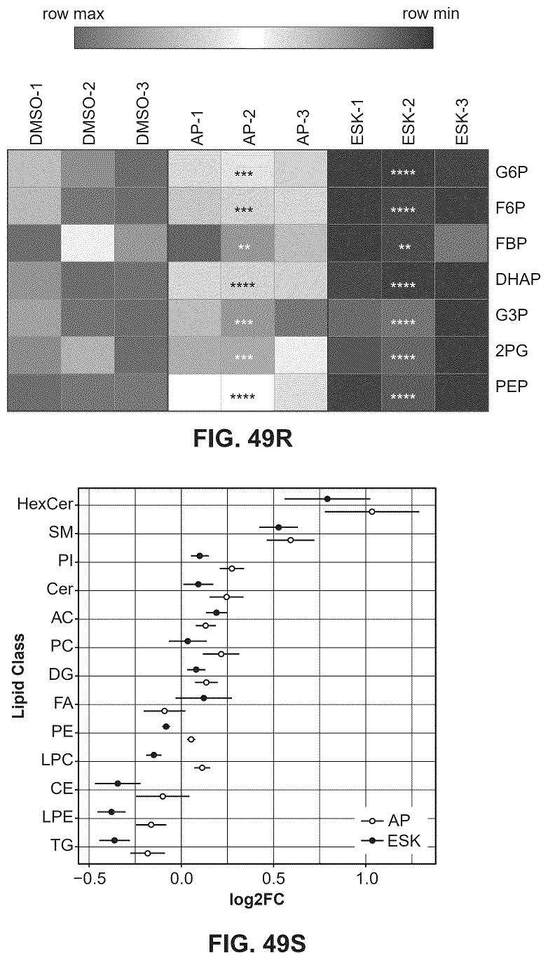

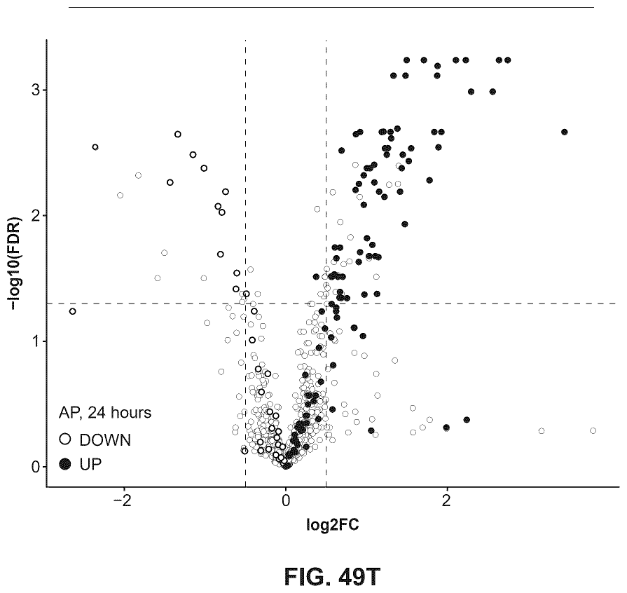

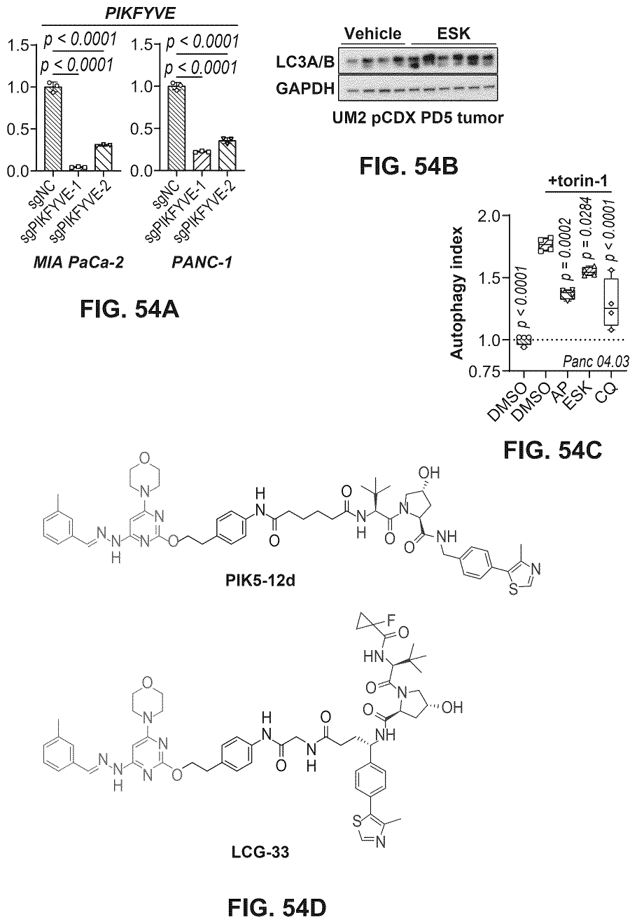

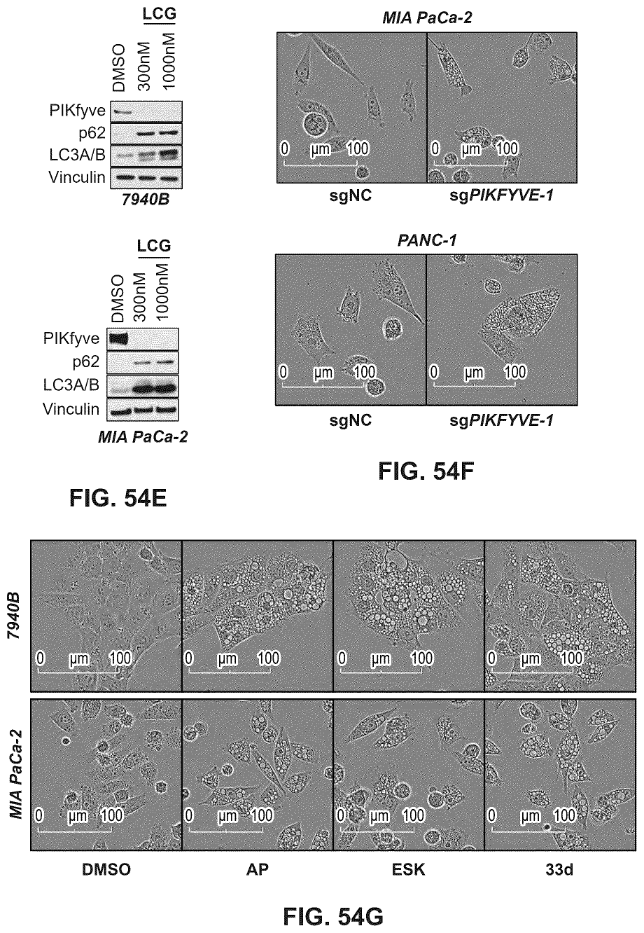

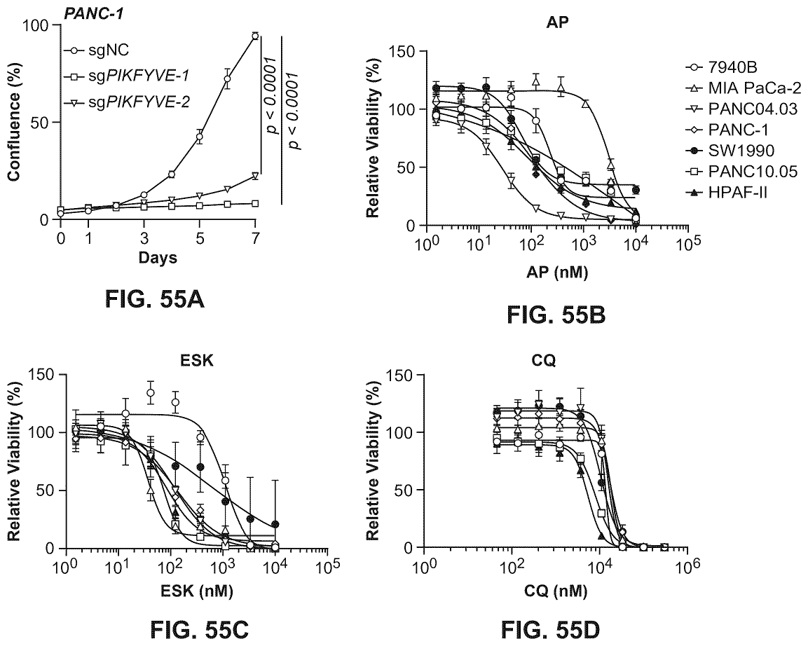

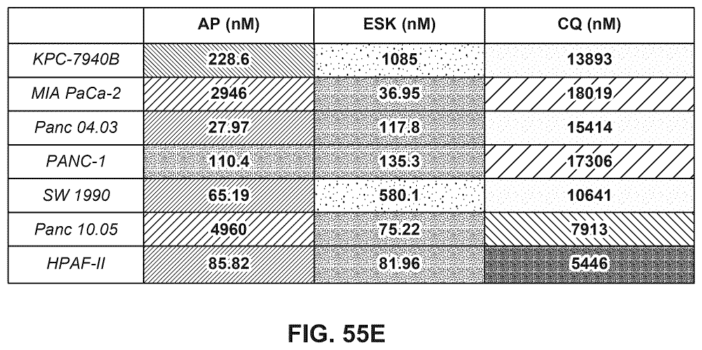

To determine the molecular effects of PIKfyve inhibition on PDAC cells, a battery of methods to perturb PIKfyve was employed. First, CRISPR interference (CRISPRi) was utilized to decrease PIKfyve transcript (FIG. 54A) and protein levels (FIG. 49A) in the human PDAC cell lines MIA PaCa-2 and PANC-1 using two independent single guide RNAs (sgRNAs). PIKfyve knockdown also increased the LC3A/B-II to LC3A/B-I ratio and increased p62 (SQSTM1) levels, suggesting an inhibition of autophagic flux (FIG. 49A), consistent with data from previous reports. Qiao, Y. et al., Nat. Cancer. 2021:2: 978-993; Bao, Y. et al., Proc. Natl. Acad. Sci. U.S.A. 2023:120. Pharmacological inhibition of PIKfyve with apilimod or ESK981 also showed similar effects in 7940B cells (murine KPC) and Panc 04.03 (human PDAC) cells as well as the UM-2 primary CDX tumors described in (FIG. 48, FIG. 49B, FIG. 54B). As an orthogonal method to validate that PIKfyve inhibition decreases autophagic flux, the GFP-LC3-RFP-LC3 ΔG autophagic flux probe was employed. Kaizuka, T. et al., Mol. Cell. 2016:64: 835-849. Treatment with apilimod, ESK981, or chloroquine decreased basal autophagic flux, as well as autophagic flux induced by mTORC inhibition with torin-1 (FIG. 49C, FIG. 54C). Similarly consistent with previous work, visual analysis of PIKfyve knockdown cells revealed a lysosomal vacuolization phenotype, which was evident within four hours of drug treatment (FIG. 54D,E). Importantly, like our tumor studies, PIKfyve knockdown substantially slowed the growth of PDAC cells (FIG. 49D, FIG. 55A), and PIKfyve inhibition decreased PDAC cell viability with half-maximal inhibitory concentrations (IC₅₀) in the nanomolar ranges for most cell lines (FIG. 55B,C). Lysosome inhibition by chloroquine treatment also decreased PDAC cell viability (FIG. 55D); however, the IC₅₀s were much higher for chloroquine than apilimod or ESK981 in the same PDAC cell lines (FIG. 49E, FIG. 55E). Taken together, these data illustrate that PIKfyve plays a crucial role in regulating autophagy and lysosomal homeostasis, and PIKfyve inhibition impairs PDAC proliferation.

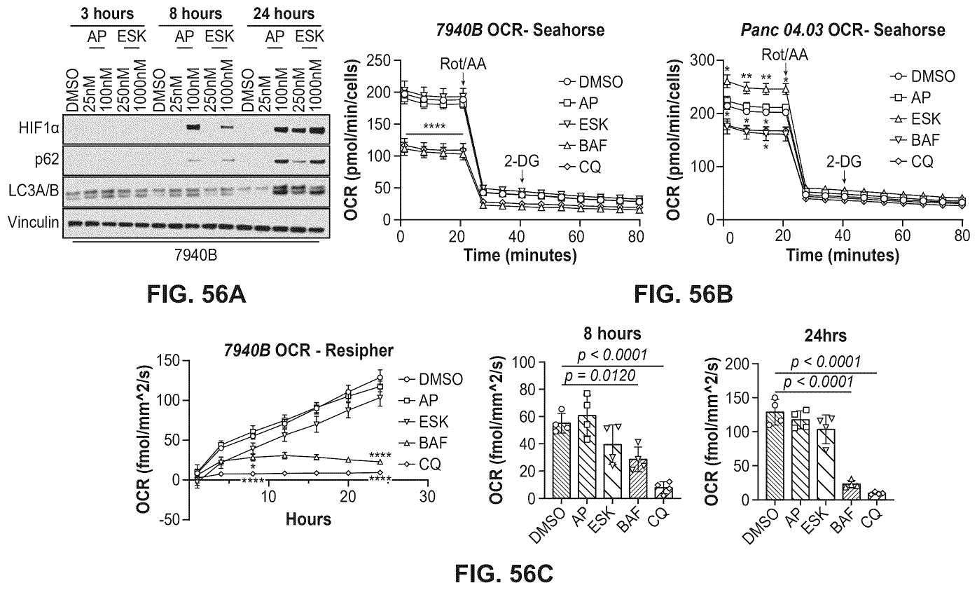

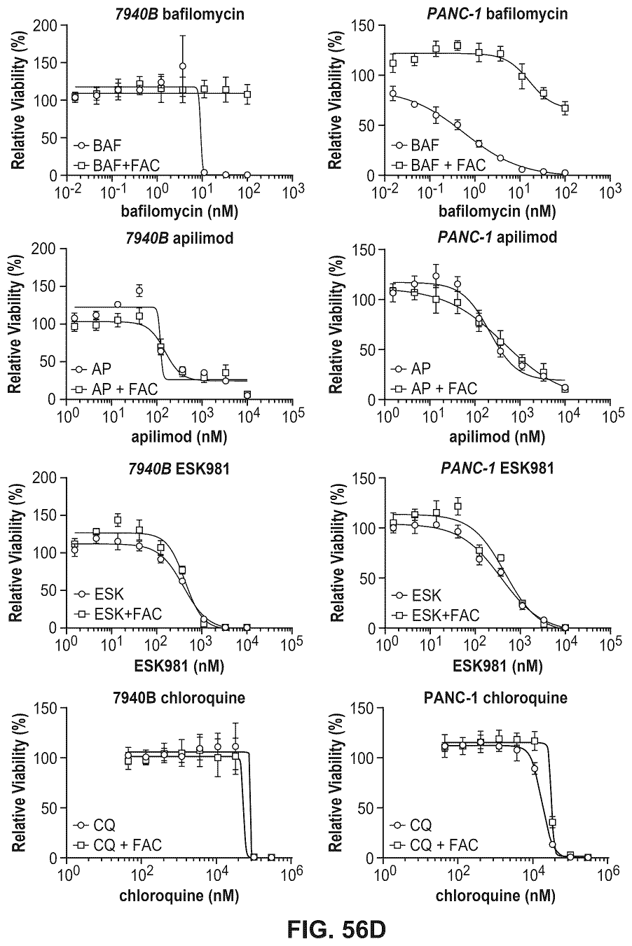

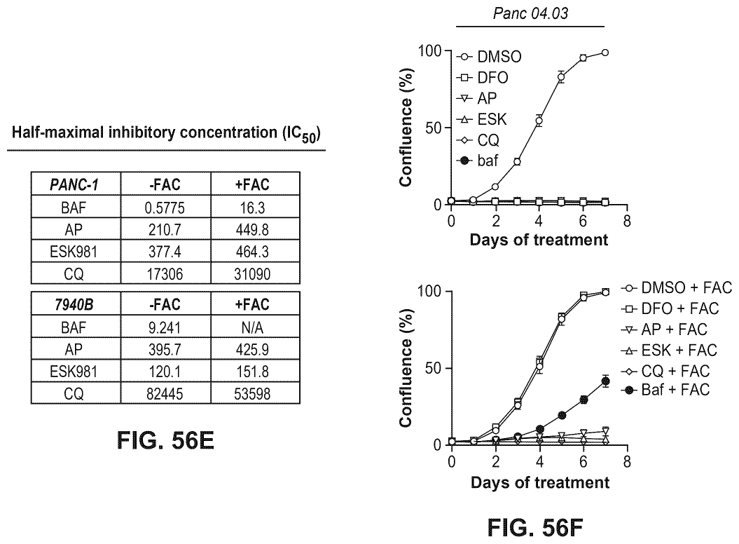

PDAC is known to utilize autophagy and lysosomal processes to promote iron homeostasis and allow for mitochondrial respiration; therefore, whether PIKfyve inhibition decreased PDAC cell proliferation through a similar mechanism was investigated. Mukhopadhyay, S. et al., Sci. Adv. 2023:9; Mancias, J. D. et al., Nature 2014:509; Weber, R. A. et al., Mol. Cell. 2020:77: 645-655. PIKfyve inhibition stabilized HIF1α upon eight hours of treatment (FIG. 56A), consistent with the effect of iron deprivation due to disrupting autophagy. However, PIKfyve inhibition did not decrease basal oxygen consumption rate (OCR) in 7940B or Panc 04.03 cells, contrasting the activity of chloroquine and bafilomycin A1, the other autophagy and lysosomal inhibitors tested (FIG. 56B). Consistent with this, PIKfyve inhibition had no impact on OCR through 24 hours of treatment, compared to chloroquine and bafilomycin A1, which significantly decreased OCR in 7940B cells starting from eight hours (FIG. 56C). To confirm that PIKfyve inhibition does not decrease PDAC cell proliferation through disrupting iron homeostasis, rescue of PDAC cells from PIKfyve inhibition was attempted using ferric ammonium citrate (FAC). While the antiproliferative effects of bafilomycin A1 were drastically attenuated by addition of FAC, we did not see a similar effect with PIKfyve inhibitors (FIG. 56D,F). Overall, these data suggest that autophagy and lysosomal perturbation through PIKfyve inhibition does not decrease PDAC proliferation by disrupting iron homeostasis and mitochondrial respiration but, rather, occurs through a distinct mechanism.

### Example 7

### PIKfyve Inhibition Creates a Synthetic Lethality of De Novo Lipid Synthesis in PDAC Cells

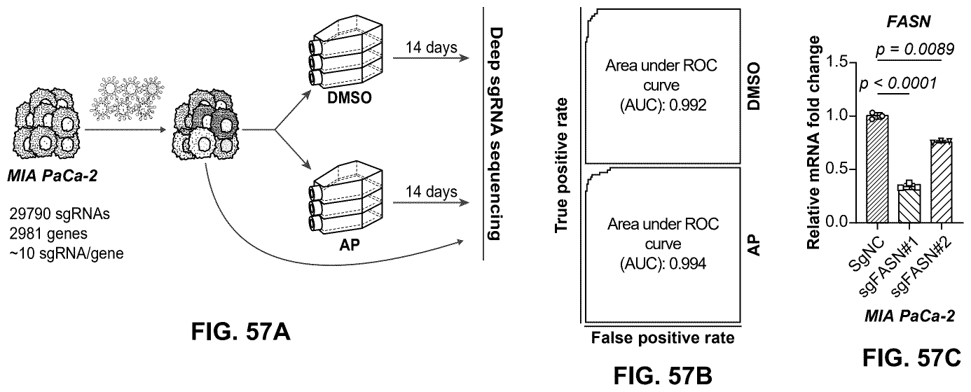

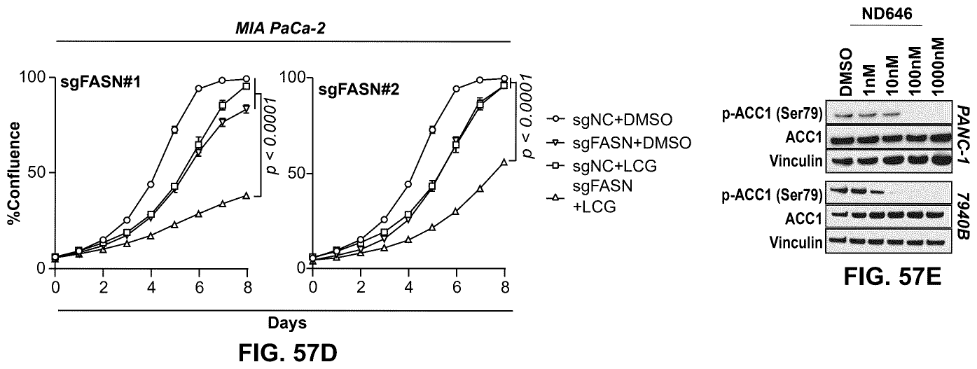

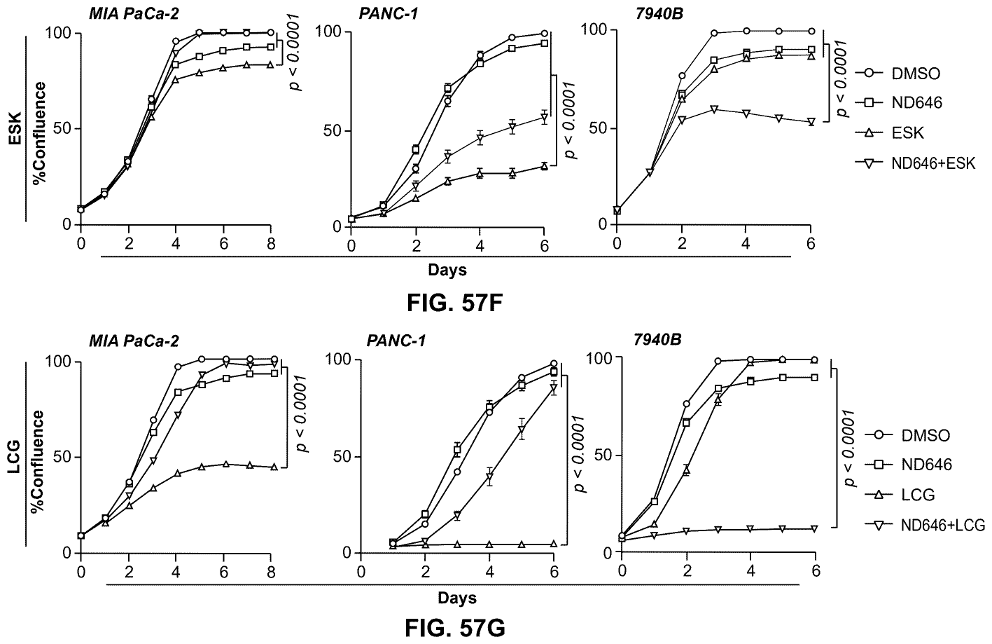

To assess the functionally relevant metabolic roles of PIKfyve in PDAC in an unbiased manner, a metabolism-focused CRISPR screen was employed in MIA PaCa-2 cells treated with apilimod (FIG. 57A). This screen accurately discriminated against core essential and non-essential genes, validating its biological relevance and consistency (FIG. 57B). Interestingly, the most significantly depleted sgRNAs targeted genes core to the de novo fatty acid synthesis and elongation pathways, namely FASN, ACACA, SLC25A1, and HSD17B12 (FIG. 49F,G). In contrast, ACOX1, which completes the first step of lipid beta-oxidation, was the target of some of the most significantly enriched sgRNAs in the screen (FIG. 49F). Additionally, no cholesterol-specific genes were among the significant hits, suggesting that de novo fatty acid synthesis was a specific, functionally relevant synthetic essentiality of MIA PaCa-2 cells upon PIKfyve inhibition (FIG. 49H). To validate this screen, CRISPRi-mediated knockdown of FASN in MIA PaCa-2 cells was employed. It was found that FASN knockdown with two independent sgRNAs (FIG. 57C) sensitized cells to apilimod (FIG. 49I). As an orthogonal validation, ND646, which is an inhibitor of ACC1 (protein name of ACACA), was utilized. After confirming on-target effects of ND646 using immunoblots (FIG. 49J, FIG. 57D, it was observed that ND646 similarly sensitized PDAC cells to apilimod (FIG. 49J) and ESK981 (FIG. 57E) using MIA PaCa-2, PANC-1, and 7940B cell lines. These data suggest that upon PIKfyve inhibition, PDAC cells become reliant on the de novo fatty acid synthesis pathway to proliferate.

### Example 8

### PIKfyve Inhibition Promotes the Upregulation of De Novo Lipid Synthesis in PDAC Cells

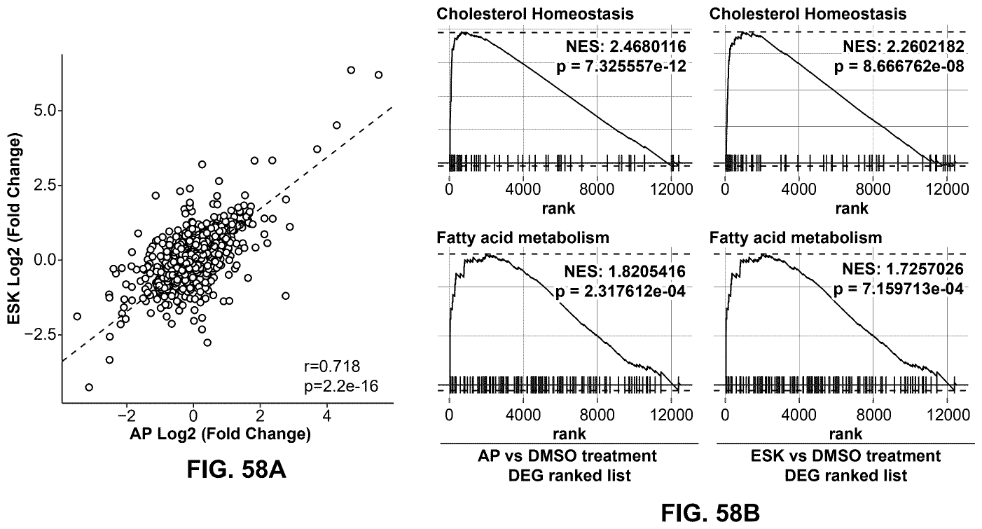

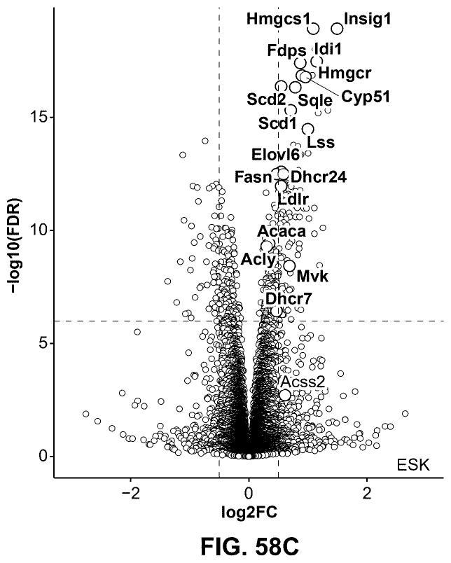

Given that PIKfyve inhibition obligates PDAC cells to maintain expression and function of the de novo fatty acid synthesis pathway, it was assessed whether PIKfyve perturbation caused upregulation of this pathway. Utilizing RNA-seq in 7940B cells, it was determined that an eight-hour treatment of apilimod or ESK981 induced concordant gene expression changes (FIG. 58A), and the most upregulated pathways were related to cholesterol homeostasis, MTORC1 signaling, and fatty acid metabolism (FIG. 49K, FIG. 58B). Additionally, most of the top upregulated genes were targets of transcription factor sterol regulatory element binding transcription factor 1 (SREBP1), a key regulator of lipogenesis (FIG. 49L, FIG. 58C). Reed, B. D. et al., PLoS Genet 2008:4. Accordingly, it was confirmed that eight hours of PIKfyve inhibition also activated SREBP1 by post-translational cleavage (FIG. 49M). Importantly, FASN was upregulated upon PIKfyve knockdown (FIG. 49N,O), and both FASN and ACC1 were upregulated upon PIKfyve inhibition at the transcript (FIG. 49P) and protein levels (FIG. 49Q). Taken together, these results illustrate that PDAC cells upregulate a lipogenic transcriptional program in response to PIKfyve inhibition.

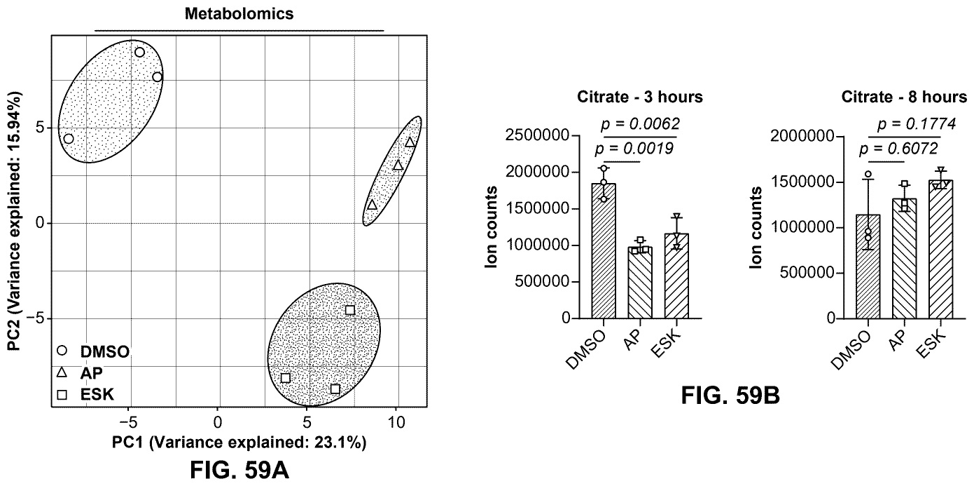

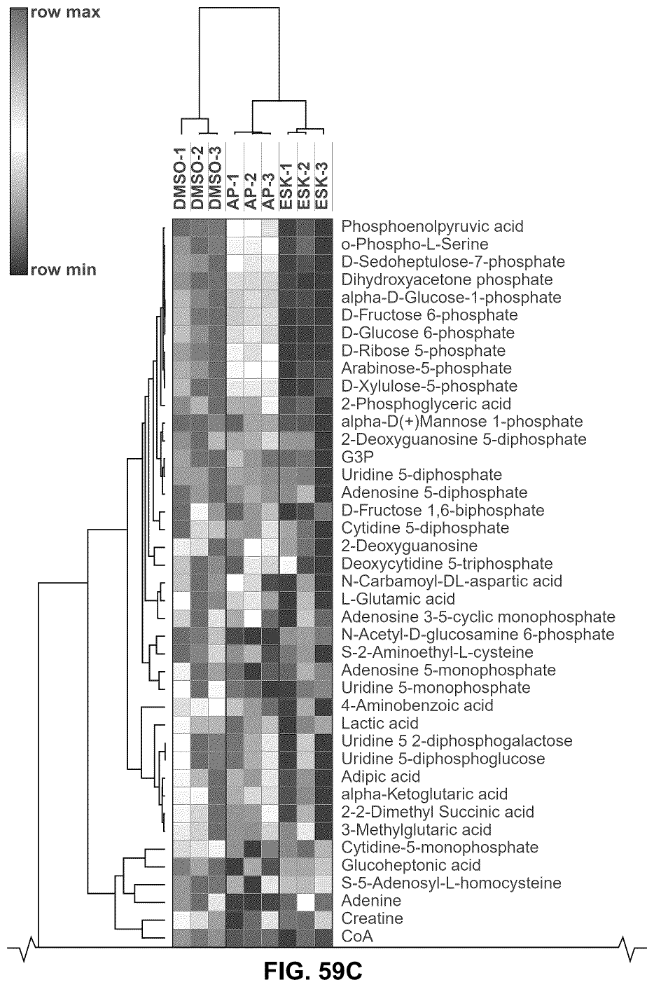

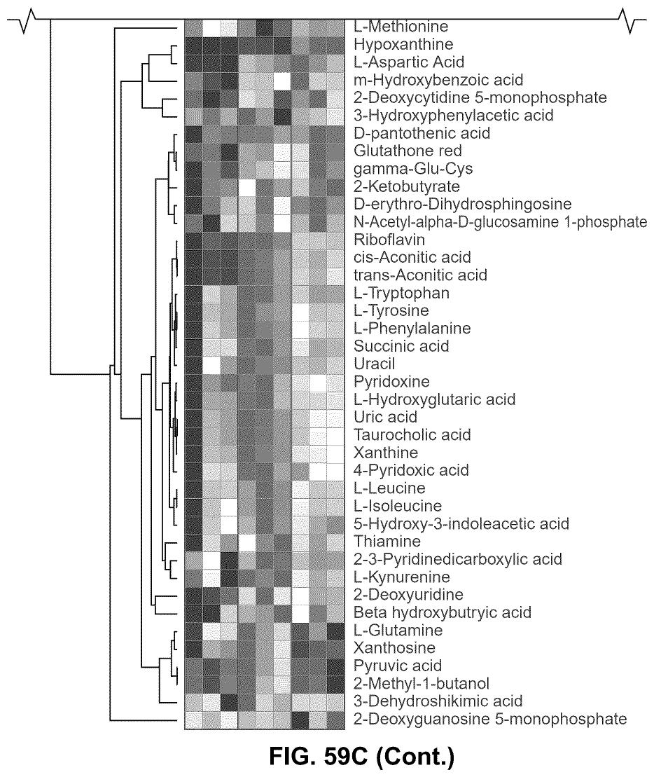

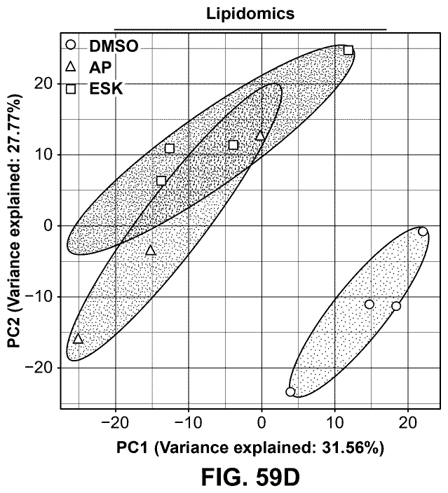

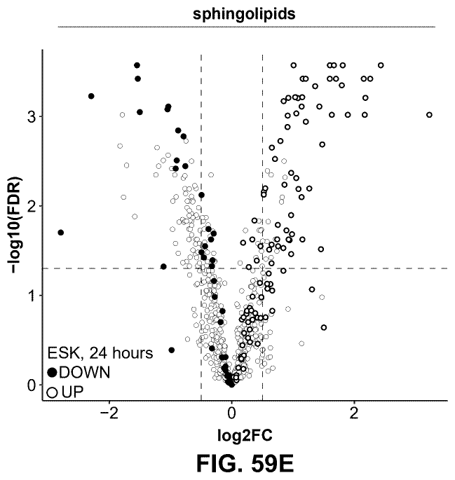

To determine whether the lipogenic transcriptional program translated to a metabolic phenotype, metabolic analyses on 7940B cells was employed. PIKfyve inhibition, using apilimod or ESK981 treatment, was observed to induce a similar metabolic landscape (FIG. 59A-C), which featured a decrease in citrate at three hours (FIG. 59B) followed by a dramatic depletion of glycolytic metabolites at eight hours of treatment (FIG. 49R). Given that the citrate transporter SLC25A1 was also a top hit in the CRISPR screen (FIG. 49H), it was hypothesized that the glycolytic metabolites were being utilized to generate citrate and shunted into de novo lipid synthesis. To verify this, targeted lipidomics was performed and showed that PIKfyve inhibition, whether through apilimod or ESK981 treatment, induced significant changes in the cellular lipid landscape in 7940B cells (FIG. 59D). Lipid species were then grouped into their respective classes and it was determined that hexosylceramides (HexCer), sphingomyelin (SM), and ceramide (Cer) were three of the top four upregulated lipid classes (FIG. 49S). These classes, all sphingolipids, contained the majority of the significantly upregulated lipid species (FIG. 49T, FIG. 59E), suggesting that PIKfyve inhibition chiefly impacts sphingolipid synthesis in PDAC cells. These data suggest that upon PIKfyve loss of function, PDAC cells are forced to increase de novo lipid synthesis and accumulate sphingolipids as a survival mechanism.

### Example 9

### KRAS-MAPK Regulates De Novo Lipid Biosynthesis in PDAC

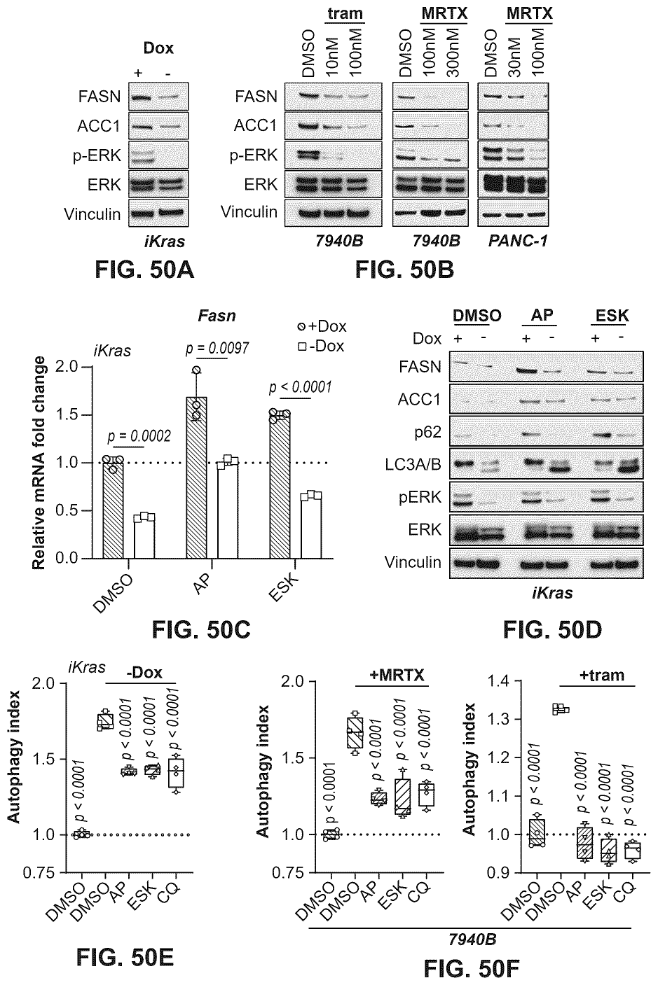

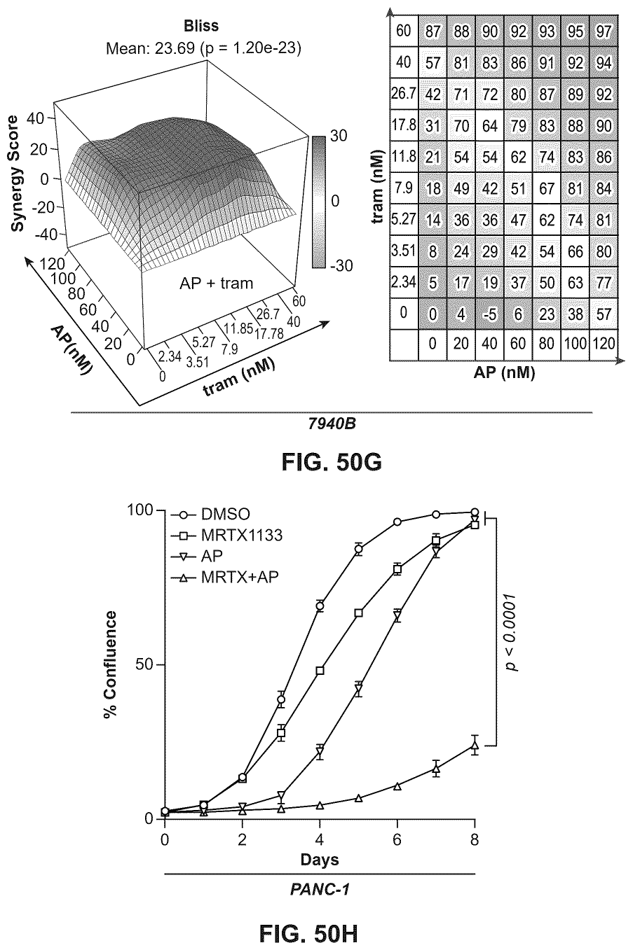

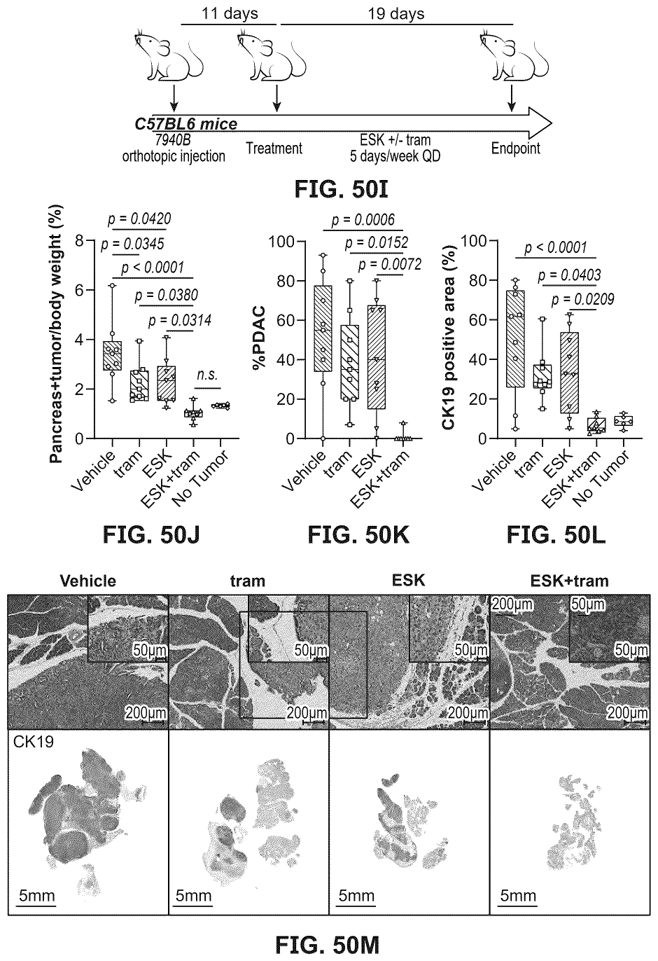

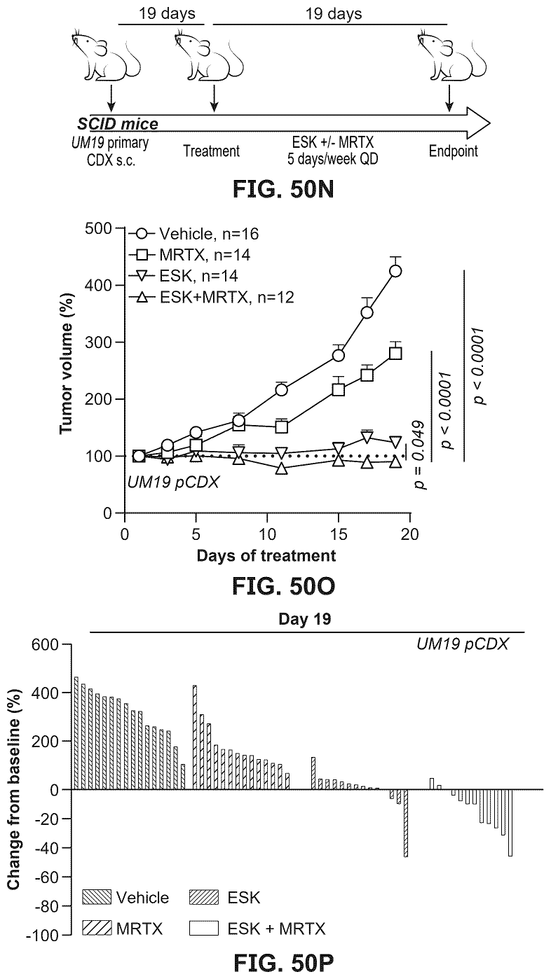

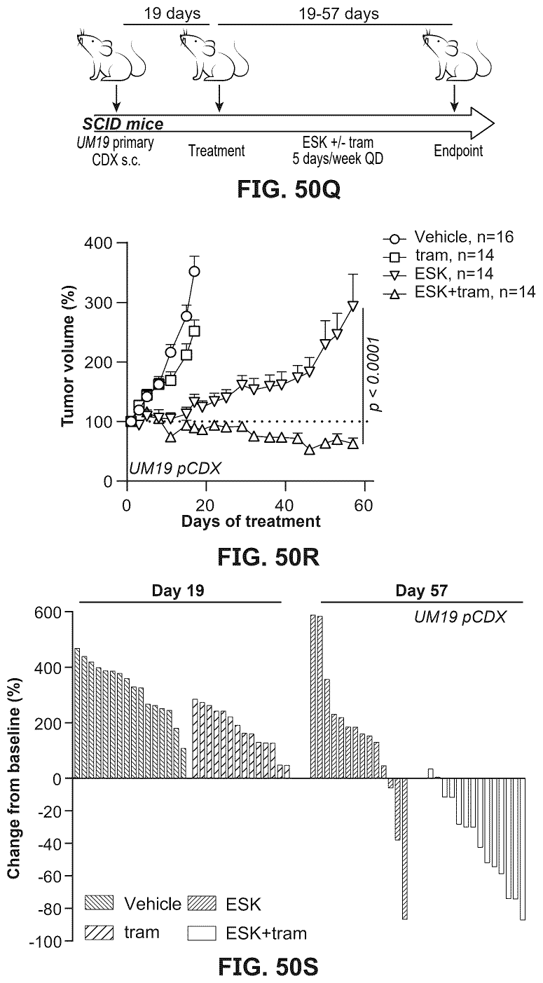

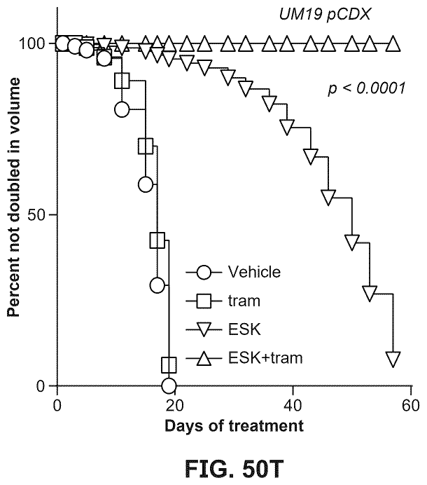

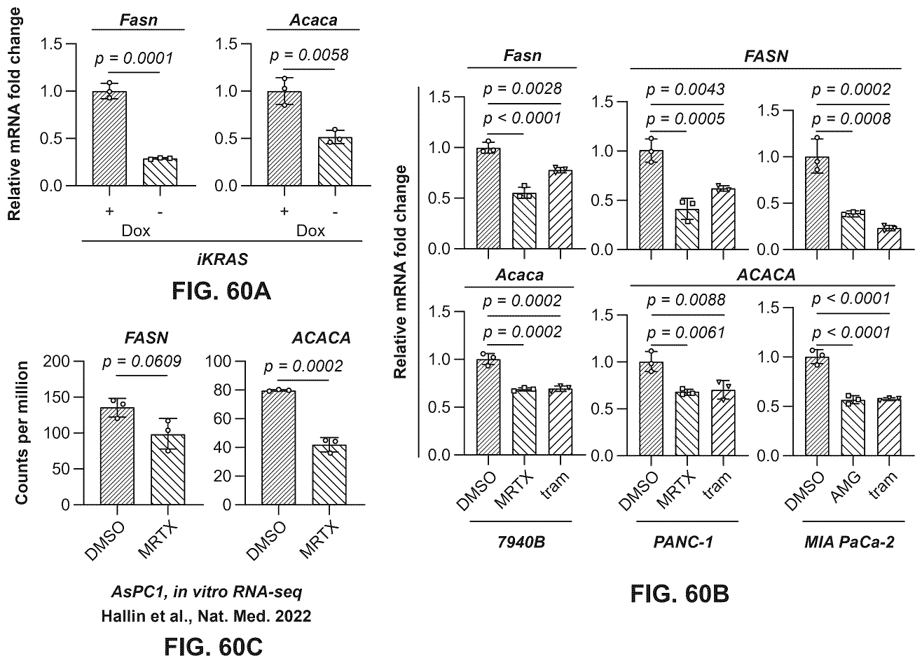

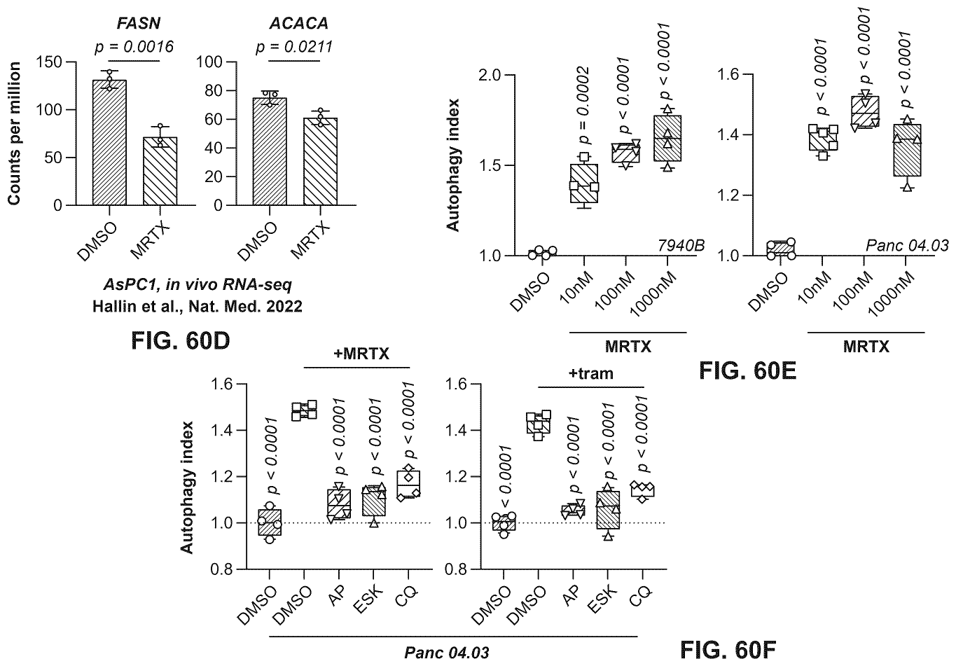

To identify avenues to possibly leverage the synthetic lethality of PIKfyve and de novo fatty acid synthesis, it was sought to determine drivers of FASN and ACACA transcription. KRAS is known to be a core driver of metabolic homeostasis in PDAC through MAPK signaling; thus, it was important to determine whether KRAS-MAPK signaling drives FASN and ACACA expression Ying, H. et al., Cell. 2012:149: 656-670. Employing an inducible Kras^{G12D} cell line, iKras 9805 (iKras), it was observed that doxycycline withdrawal (Kras OFF) decreased Fasn and Acaca expression at the transcript (FIG. 60A), and protein level (FIG. 50A) Collins, M. A. et al., J. Clin. Invest. 2012:122: 639-653. Further, after eight-hours of treatment with MRTX1133, a KRAS^{G12D} inhibitor, AMG510, a KRAS^{G12C} inhibitor, or trametinib, a MEK inhibitor, decreased transcription of FASN and ACACA (FIG. 60B) in PDAC cell lines with the relevant KRAS mutation was observed. This was reflected by a decrease in protein level after 48 hours of treatment (FIG. 50B). These data are concordant with previously published RNA-seq data suggesting that MRTX1133 treatment decreases FASN and ACACA transcripts in AsPC1 cells in vitro (FIG. 60C) and in vivo (FIG. 60D) Hallin, J. et al., Nat. Med. 2022:28: 2171-2182. Moreover, these data also illustrate that KRAS-MAPK signaling regulates FASN and ACACA expression in PDAC.

To directly assess the effects of dual inhibition of PIKfyve and KRAS on FASN and ACC1, iKras cells were treated with PIKfyve inhibitors after incubation with or without doxycycline. As expected, PIKfyve inhibition increased the transcription of FASN, while Kras OFF prevented this increase (FIG. 50C). In a similar fashion, PIKfyve inhibition increased the protein levels of FASN and ACC1, while Kras OFF again prevented this increase (FIG. 50D). This indicates that KRAS-MAPK inhibition blocks FASN and ACACA expression, synthetically critical genes in PDAC upon PIKfyve inhibition.

### Example 10

### Concurrent Perturbation of PIKfyve and KRAS-MAPK Creates Metabolic Conflict of Autophagy Regulation

Recent studies have revealed that PDACs upregulate and depend on autophagy to maintain metabolic homeostasis upon KRAS-MAPK signaling inhibition Kinsey, C. G. et al., Nat. Med. 2019:25: 620-627; Bryant, K. L. et al., Nat. Med. 2019:25: 628-640; Lee, C. S. et al., Proc. Natl. Acad. Sci. U.S.A. 2019:116: 4508-4517. Utilizing the autophagic flux probe, it was confirmed that PDAC cells upregulate autophagy upon acute mutant KRAS^{G12D} inhibition with MRTX-1133 (FIG. 60E). Knowing that PIKfyve inhibition disrupts autophagic flux, it was sought to determine whether PIKfyve inhibition could also leverage this synthetic metabolic dependency. Since, Kras OFF induced an increase in the LC3-II to LC3-I ratio, it was observed that this ratio was maintained when PIKfyve inhibitors were added (FIG. 50D). Additionally, p62 was shown to decrease upon Kras OFF, but was observed to increase upon PIKfyve inhibition (FIG. 4D). The p62 was observed to less dramatically change with both Kras OFF and PIKfyve inhibition (FIG. 50D). This suggests that PIKfyve inhibition and Kras OFF exert opposing effects on autophagic flux. This was validated using the autophagic flux probe assay in iKras cells, which showed an increase in autophagic flux upon Kras OFF that was attenuated with PIKfyve inhibition or chloroquine treatment (FIG. 50E). Pharmacological inhibition of KRAS-MAPK using MRTX1133 or trametinib also induced autophagic flux that was blocked upon PIKfyve inhibition in 7940B (FIG. 50F) and Panc 04.03 cells (FIG. 60F). Altogether, this suggests that concurrent PIKfyve and KRAS-MAPK inhibition drives PDAC into a state of metabolic conflict regarding its regulation of autophagic flux.

### Example 11

### Dual Inhibition of PIKfyve and KRAS-MAPK Synergistically Suppresses PDAC Growth

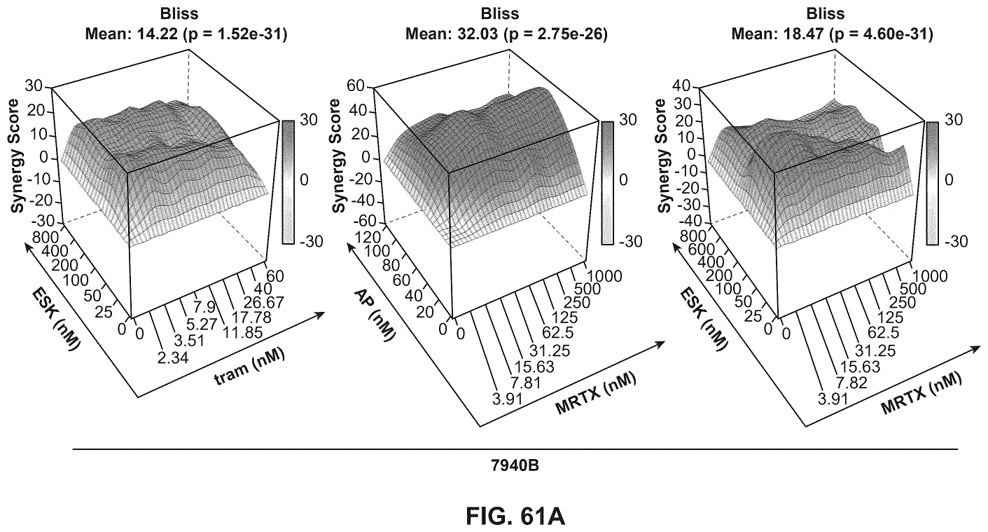

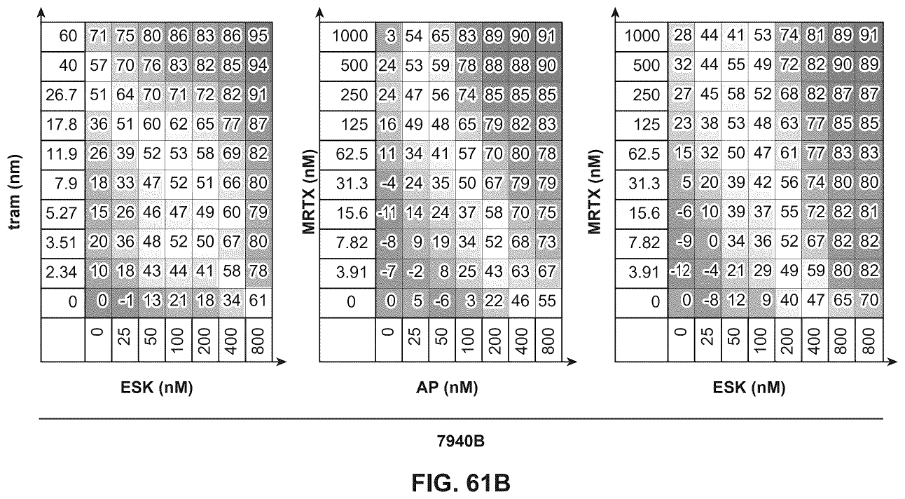

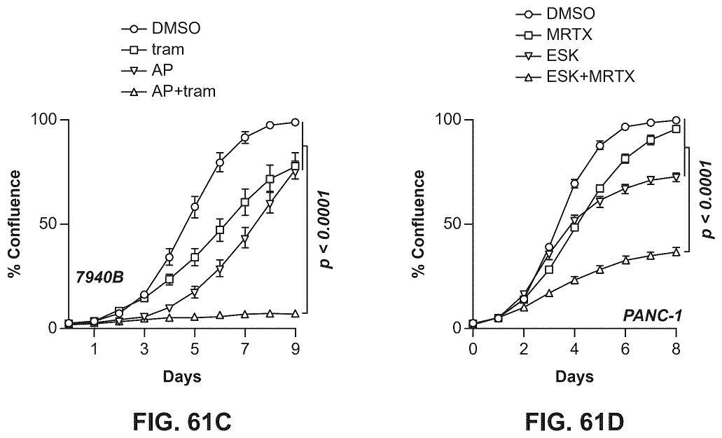

Next it was assessed whether the metabolic crises elicited by simultaneous inhibition of PIKfyve and KRAS-MAPK could be utilized to inhibit PDAC cell proliferation. Synergy assays confirmed that any combination of PIKfyve inhibition, using apilimod or ESK981, and KRAS-MAPK inhibition, using MRTX1133 or trametinib, resulted in striking synergistic effects, decreasing PDAC cell proliferation and viability (FIG. 50G,H, FIG. 61A-D).

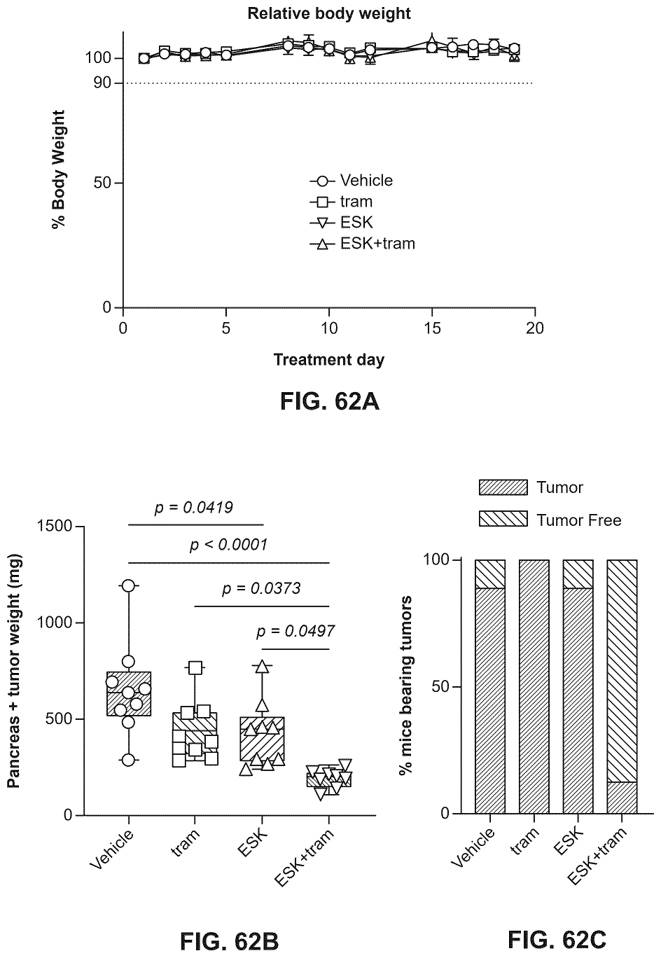

To determine the efficacy of combining PIKfyve and KRAS-MAPK inhibitors as a therapeutic strategy for PDAC, a syngeneic orthotopic preclinical model was utilized (FIG. 50I). Importantly, treatment with ESK981 and/or trametinib did not impact mouse body weight throughout the treatment course (FIG. 62A). Upon endpoint analysis, it was observed that the mice treated with ESK981 and trametinib had significantly lighter pancreata (FIG. 62B), comparable to those found in age-matched, non-tumor bearing mice, while the individual treatments had more modest effects (FIG. 50J). Histopathological evaluation with H&E and CK19 corroborated this, revealing no evidence of PDAC in seven out of eight mice treated with both ESK981 and trametinib, while either treatment alone exhibited only marginal effects (FIG. 50K-M). Taken together, these data illustrate that combination therapy of a PIKfyve inhibitor and MEK inhibitor eliminated tumor burden in an immunocompetent orthotopic PDAC model (FIG. 61C).

Next, utilizing UM-19, a primary KRAS^{G12D} PDAC CDX (pCDX) model, this therapeutic strategy was further tested in a human system (FIG. 50N). Combination treatment of ESK981 and MRTX1133 significantly improved the efficacy of either treatment alone throughout the treatment duration (FIG. 50O). At endpoint, the combination induced regression in nearly all tumors, while each individual treatment only had modest effects (FIG. 50P). Further, combining ESK981 and trametinib (FIG. 50Q) induced substantial and durable regression in nearly all tumors, even when the tumors were able to adapt and outgrow ESK981 or trametinib therapy alone (FIG. 50R). At endpoint, most of the tumors treated with the combination were still smaller than their original size (FIG. 50S). Ultimately, the combination prevented any tumor from doubling throughout the duration of the experiment, while nearly all tumors from the other treatment groups were shown to double or more in size (FIG. 50T).

In sum, these data demonstrate that KRAS-MAPK inhibition creates a synthetic vulnerability to PIKfyve inhibition in vitro and in vivo. Unlike previous efforts to target autophagy in PDAC, ESK981 has vastly superior pharmacological properties. Further, the arrival of KRAS inhibitors provides exciting context to explore this combination in the clinic, noting the safety profile of the combination in our studies.

### Example 12

### Discussion

Targeting lysosome function and the autophagic pathway as a therapeutic strategy has shown promise preclinically, given the known metabolic vulnerabilities of PDAC. Further enhancing this concept was the important finding that PDAC utilizes autophagy to compensate for KRAS-MAPK inhibition. Kinsey, C. G. et al., Nat. Med. 2019:25: 620-627; Bryant, K. L. et al., Nat. Med. 2019:25: 628-640; Lee, C. S. et al., Proc. Natl. Acad. Sci. U.S.A. 2019:116: 4508-4517. However, hydroxychloroquine, the only clinical-grade compound available to target these pathways, has had limited efficacy. Karasic, T. B. et al., JAMA Oncol. 2019:5: 993-998; Zeh, H. J. et al., Clin. Cancer Res. 202:26: 3126-3134. In addition, hydroxychloroquine, and its predecessor chloroquine, do not have a definitive molecular target, making it suboptimal for systematic pharmacological development. Schrezenmeier, E. and Dorner, T. Nat. Rev. Rheumatol. 2020:16: 155-166. In this study, PIKfyve, a lipid kinase known for its important roles in lysosomal function, was nominated as a druggable target to leverage PDAC's metabolic vulnerabilities of nutrient scavenging and recycling through the lysosome. In previous studies, PIKfyve knockout or inhibition with ESK981 was shown to substantially reduce malignant transformation in PDAC development models. Further, PIKfyve inhibition dramatically decreased tumor growth in murine and human in vivo models, suggesting that PDAC relies on PIKfyve for both tumor development and growth. Taken together, these data highlight PIKfyve as the first gene involved in autophagy/lysosome function for which there exists both genetic and clinically relevant pharmacologic evidence of its viability as a therapeutic target in PDAC.

Though lysosomal processes and autophagy have long been identified as metabolic targets for PDAC, the exact roles they play in PDAC metabolic homeostasis remain unclear. Recent work using a metabolism-focused CRISPR screen in an acute T cell leukemia line with the V-ATPase inhibitor bafilomycin and ammonia have demonstrated that maintaining iron homeostasis is crucial for lysosome function. Weber, R. A. et al., Mol. Cell. 2020:77: 645-655. Multiple reports have independently verified and expanded on this concept in various models, including PDAC. Santana-Codina, N. et al., Cancer Discov. 2022:12: 2180-2197; Ravichandran, M. et al., Cancer Discov. 2022:12: 2198-2219. In this study, autophagy inhibition was approached using a well-defined target and inhibitor. Applying this to a similar CRISPR screening library in MIA PaCa-2 cells, surprisingly showed that five out of the top ten hits were genes core to the fatty acid synthesis and elongation pathway, such as FASN and ACACA (FIG. 49F-H). RNA-seq experiments further highlighted lipid metabolism as the most dramatically affected gene signatures upon PIKfyve inhibition (FIG. 49K). Taken in context, this data raises the possibility that specific methods of inhibiting lysosomal processes may have differential effects on various aspects of PDAC cell metabolism.

PIKfyve was recently shown to play a role in lipid metabolism through its role in lysosome function. Hosios, A. M. et al., Nat. Metab. 2022:4: 1792-1811. In that study, de novo fatty acid synthesis was inhibited and it was found that cells undergo increased phospholipid turnover in a lysosome- and PIKfyve-dependent process. Conversely, this work has identified that PIKfyve inhibition stimulates de novo fatty acid synthesis and elongation. Together, these studies provide independent and complementary evidence that PIKfyve plays a crucial role in maintaining lipid homeostasis in coordination with de novo fatty acid synthesis, suggesting that disruption of one pathway increases a cells' dependence on the other. Thus, a logical implication made by both studies would be that simultaneous perturbation of both pathways would lead to catastrophic metabolic dysregulation.

The therapeutic strategy of inhibiting both KRAS-MAPK and autophagy has gained considerable recent attention, including being the subject of recent clinical trials (NCT04386057, NCT04132505). Kinsey, C. G. et al., Nat. Med. 2019:25: 620-627; Bryant, K. L. et al., Nat. Med. 2019:25: 628-640; Lee, C. S. et al., Proc. Natl. Acad. Sci. U.S.A. 2019:116: 4508-4517. In the studies describing this relationship, it was identified that PDAC utilizes autophagy as an adaptive and protective mechanism to maintain metabolic homeostasis upon KRAS-MAPK inhibition. Knowing PIKfyve's role in autophagy, this directly positions PIKfyve inhibitors as alternatives to chloroquine to pair with KRAS-MAPK inhibitors for PDAC therapy (FIG. 50E,F). However, this study, shows the discovery of a mechanistically distinct rationale for dual inhibition of PIKfyve and KRAS-MAPK. Through the metabolic CRISPR screen, PIKfyve and fatty acid synthesis were identified to have a robust relationship of synthetic lethality (FIG. 49F-H). In a search for translatable methods for leveraging this novel relationship, it was hypothesized that KRAS-MAPK would drive fatty acid synthesis. It was shown that KRAS-MAPK perturbation transcriptionally downregulated key fatty acid synthesis genes FASN and ACACA (FIG. 50A-D). Positioning KRAS-MAPK inhibitors as promising combination therapies with PIKfyve inhibitors for PDAC therapy. This combination was assessed in both in vitro and in vivo preclinical models. It was found that in each case the combination exhibited dramatically more potent effects than the individual therapies, in some cases even eliminating tumor burden.

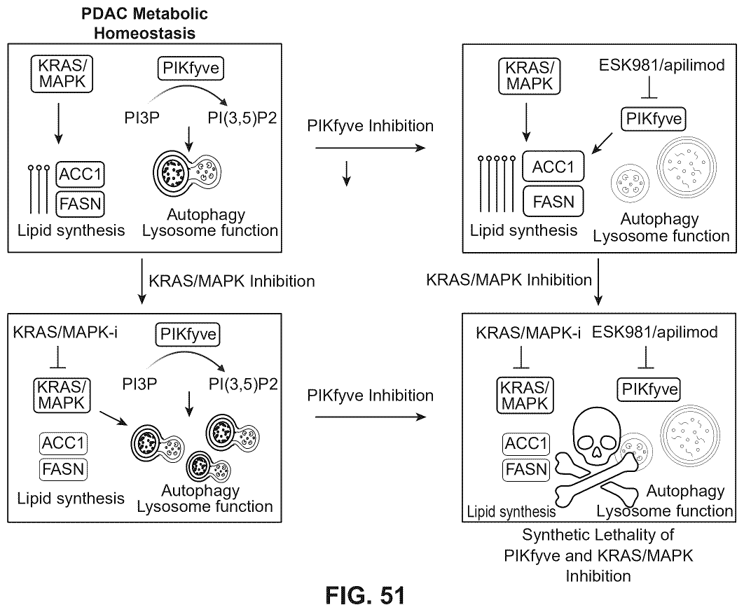

In summary, PIKfyve was shown as a therapeutic target to disrupt PDAC lysosomal function, a unique metabolic dependency of PDAC. Supporting this, it was found that PIKfyve knockout or inhibition alone decreased PDAC development in the KPC murine model. Mechanistically, this novel relationship of synthetic lethality between PIKfyve and fatty acid synthesis was identified and characterized. Further, it was shown that PIKfyve and KRAS-MAPK have a bidirectional synthetic lethality relationship. First, PIKfyve inhibition disrupts PDAC autophagy and lysosomal function, requiring PDAC to upregulate and depend on de novo fatty acid synthesis through FASN and ACC1. Second, KRAS-MAPK inhibition decreases expression of FASN and ACC1 and increases PDAC utilization and reliance on autophagy. Lastly, dual inhibition of PIKfyve and KRAS-MAPK drives PDAC into a metabolic crisis (FIG. 51). Given the rapidly evolving landscape of mutant-KRAS, pan-(K)RAS, and MAPK pathway inhibitor development, this highlights the combination of PIKfyve and KRAS-MAPK inhibitors as an promising and rapidly translatable therapeutic strategy for PDAC. Skoulidis, F. et al., N. Engl. J. Med. 2021:384: 2371-2381; Hallin, J. et al., Nat. Med. 2022:28: 2171-2182; Wasko, U. N. et al., bioRxiv, 2023; Kim, D. et al., Nature 2023:619: 160-166.

### REFERENCES

1 Biancur, D. E. et al. Functional Genomics Identifies Metabolic Vulnerabilities in Pancreatic Cancer. Cell Metab 33, 199-210 e198, doi:10.1016/j.cmet.2020.10.018 (2021).
2 Halbrook, C. J., Lyssiotis, C. A., Pasca di Magliano, M. & Maitra, A. Pancreatic cancer: Advances and challenges. Cell 186, 1729-1754, doi:10.1016/j.cell.2023.02.014 (2023).
3 Yang, S. et al. Pancreatic cancers require autophagy for tumor growth. Genes Dev 25, 717-729, doi:10.1101/gad.2016111 (2011).
4 Davidson, S. M. et al. Direct evidence for cancer-cell-autonomous extracellular protein catabolism in pancreatic tumors. Nat Med 23, 235-241, doi:10.1038/nm.4256 (2017).
5 Commisso, C. et al. Macropinocytosis of protein is an amino acid supply route in Ras-transformed cells. Nature 497, 633-637, doi:10.1038/nature12138 (2013).
6 Piffoux, M., Eriau, E. & Cassier, P. A. Autophagy as a therapeutic target in pancreatic cancer. Br J Cancer 124, 333-344, doi:10.1038/s41416-020-01039-5 (2021).
7 Rivero-Rios, P. & Weisman, L. S. Roles of PIKfyve in multiple cellular pathways. Curr Opin Cell Biol 76, 102086, doi:10.1016/j.ceb.2022.102086 (2022).
8 Wood, L. D., Canto, M. I., Jaffee, E. M. & Simeone, D. M. Pancreatic Cancer: Pathogenesis, Screening, Diagnosis, and Treatment. Gastroenterology 163, 386-402 e381, doi:10.1053/j.gastro.2022.03.056 (2022).
9 Kerk, S. A., Papagiannakopoulos, T., Shah, Y. M. & Lyssiotis, C. A. Metabolic networks in mutant KRAS-driven tumours: tissue specificities and the microenvironment. Nat Rev Cancer 21, 510-525, doi:10.1038/s41568-021-00375-9 (2021).
10 Ying, H. et al. Oncogenic Kras maintains pancreatic tumors through regulation of anabolic glucose metabolism. Cell 149, 656-670, doi:10.1016/j.cell.2012.01.058 (2012).
11 Nwosu, Z. C. et al. Uridine-derived ribose fuels glucose-restricted pancreatic cancer. Nature 618, 151-158, doi:10.1038/s41586-023-06073-w (2023).
12 Yang, A. et al. Autophagy is critical for pancreatic tumor growth and progression in tumors with p53 alterations. Cancer Discov 4, 905-913, doi:10.1158/2159-8290.CD-14-0362 (2014).
13 Kamphorst, J. J. et al. Human pancreatic cancer tumors are nutrient poor and tumor cells actively scavenge extracellular protein. Cancer Res 75, 544-553, doi:10.1158/0008-5472.CAN-14-2211 (2015).
14 Sousa, C. M. et al. Pancreatic stellate cells support tumour metabolism through autophagic alanine secretion. Nature 536, 479-483, doi:10.1038/nature19084 (2016).
15 Banh, R. S. et al. Neurons Release Serine to Support mRNA Translation in Pancreatic Cancer. Cell 183, 1202-1218 e1225, doi:10.1016/j.cell.2020.10.016 (2020).
16 Zhu, Z. et al. Tumour-reprogrammed stromal BCAT1 fuels branched-chain ketoacid dependency in stromal-rich PDAC tumours. Nat Metab 2, 775-792, doi:10.1038/s42255-020-0226-5 (2020).
17 Recouvreux, M. V. et al. Glutamine mimicry suppresses tumor progression through asparagine metabolism in pancreatic ductal adenocarcinoma. Nat Cancer, doi:10.1038/s43018-023-00649-1 (2023).
18 Encarnacion-Rosado, J. et al. Targeting pancreatic cancer metabolic dependencies through glutamine antagonism. Nat Cancer, doi:10.1038/s43018-023-00647-3 (2023).
19 Encarnacion-Rosado, J. & Kimmelman, A. C. Harnessing metabolic dependencies in pancreatic cancers. Nat Rev Gastroenterol Hepatol 18, 482-492, doi:10.1038/s41575-021-00431-7 (2021).
20 Yang, A. et al. Autophagy Sustains Pancreatic Cancer Growth through Both Cell-Autonomous and Nonautonomous Mechanisms. Cancer Discov 8, 276-287, doi:10.1158/2159-8290.CD-17-0952 (2018).
21 Yang, A. & Kimmelman, A. C. Inhibition of autophagy attenuates pancreatic cancer growth independent of TP53/TRP53 status. Autophagy 10, 1683-1684, doi:10.4161/auto.29961 (2014).
22 White, E. Deconvoluting the context-dependent role for autophagy in cancer. Nat Rev Cancer 12, 401-410, doi:10.1038/nrc3262 (2012).
23 Kimmelman, A. C. & White, E. Autophagy and Tumor Metabolism. Cell Metab 25, 1037-1043, doi:10.1016/j.cmet.2017.04.004 (2017).
24 Mukhopadhyay, S. et al. Autophagy supports mitochondrial metabolism through the regulation of iron homeostasis in pancreatic cancer. Sci Adv 9, eadf9284, doi:10.1126/sciadv.adf9284 (2023).
25 Mancias, J. D., Wang, X., Gygi, S. P., Harper, J. W. & Kimmelman, A. C. Quantitative proteomics identifies NCOA4 as the cargo receptor mediating ferritinophagy. Nature 509, 105-109, doi:10.1038/nature13148 (2014).
26 Santana-Codina, N. et al. NCOA4-Mediated Ferritinophagy Is a Pancreatic Cancer Dependency via Maintenance of Iron Bioavailability for Iron-Sulfur Cluster Proteins. Cancer Discov 12, 2180-2197, doi:10.1158/2159-8290.CD-22-0043 (2022).
27 Yamamoto, K. et al. Autophagy promotes immune evasion of pancreatic cancer by degrading MHC-I. Nature 581, 100-105, doi:10.1038/s41586-020-2229-5 (2020).
28 Yamamoto, K., Venida, A., Perera, R. M. & Kimmelman, A. C. Selective autophagy of MHC-I promotes immune evasion of pancreatic cancer. Autophagy 16, 1524-1525, doi:10.1080/15548627.2020.1769973 (2020).
29 Karasic, T. B. et al. Effect of Gemcitabine and nab-Paclitaxel With or Without Hydroxychloroquine on Patients With Advanced Pancreatic Cancer: A Phase 2 Randomized Clinical Trial. JAMA Oncol 5, 993-998, doi:10.1001/jamaoncol.2019.0684 (2019).
30 Zeh, H. J. et al. A Randomized Phase II Preoperative Study of Autophagy Inhibition with High-Dose Hydroxychloroquine and Gemcitabine/Nab-Paclitaxel in Pancreatic Cancer Patients. Clin Cancer Res 26, 3126-3134, doi:10.1158/1078-0432.CCR-19-4042 (2020).
31 Wolpin, B. M. et al. Phase II and pharmacodynamic study of autophagy inhibition using hydroxychloroquine in patients with metastatic pancreatic adenocarcinoma. Oncologist 19, 637-638, doi:10.1634/theoncologist.2014-0086 (2014).
32 Kinsey, C. G. et al. Protective autophagy elicited by RAF-->MEK-->ERK inhibition suggests a treatment strategy for RAS-driven cancers. Nat Med 25, 620-627, doi:10.1038/s41591-019-0367-9 (2019).
33 Bryant, K. L. et al. Combination of ERK and autophagy inhibition as a treatment approach for pancreatic cancer. Nat Med 25, 628-640, doi:10.1038/s41591-019-0368-8 (2019).
34 Lee, C. S. et al. MAP kinase and autophagy pathways cooperate to maintain RAS mutant cancer cell survival. Proc Natl Acad Sci USA 116, 4508-4517, doi:10.1073/pnas.1817494116 (2019).
35 Viale, A. et al. Oncogene ablation-resistant pancreatic cancer cells depend on mitochondrial function. Nature 514, 628-632, doi:10.1038/nature13611 (2014).
36 Bryant, K. L., Mancias, J. D., Kimmelman, A. C. & Der, C. J. KRAS: feeding pancreatic cancer proliferation. Trends Biochem Sci 39, 91-100, doi:10.1016/j.tibs.2013.12.004 (2014).
37 Huffman, B. M. et al. Emerging Role of Targeted Therapy in Metastatic Pancreatic Adenocarcinoma. Cancers (Basel) 14, doi:10.3390/cancers14246223 (2022).
38 Nissley, D. V. & McCormick, F. RAS at 40: Update from the RAS Initiative. Cancer Discov 12, 895-898, doi:10.1158/2159-8290.CD-21-1554 (2022).
39 Wang, X. et al. Identification of MRTX1133, a Noncovalent, Potent, and Selective KRAS(G12D) Inhibitor. J Med Chem 65, 3123-3133, doi:10.1021/acs.jmedchem.1c01688 (2022).
40 Skoulidis, F. et al. Sotorasib for Lung Cancers with KRAS p.G12C Mutation. N Engl J Med 384, 2371-2381, doi:10.1056/NEJMoa2103695 (2021).
41 Lanman, B. A. et al. Discovery of a Covalent Inhibitor of KRAS(G12C) (AMG 510) for the Treatment of Solid Tumors. J Med Chem 63, 52-65, doi:10.1021/acs.jmedchem.9b01180 (2020).
42 Kemp, S. B. et al. Efficacy of a Small-Molecule Inhibitor of KrasG12D in Immunocompetent Models of Pancreatic Cancer. Cancer Discov 13, 298-311, doi:10.1158/2159-8290.CD-22-1066 (2023).
43 Infante, J. R. et al. A randomised, double-blind, placebo-controlled trial of trametinib, an oral MEK inhibitor, in combination with gemcitabine for patients with untreated metastatic adenocarcinoma of the pancreas. Eur J Cancer 50, 2072-2081, doi:10.1016/j.ejca.2014.04.024 (2014).
44 Hallin, J. et al. Anti-tumor efficacy of a potent and selective non-covalent KRAS(G12D) inhibitor. Nat Med 28, 2171-2182, doi:10.1038/s41591-022-02007-7 (2022).
45 Xavier, C. B., Marchetti, K. R., Castria, T. B., Jardim, D. L. F. & Fernandes, G. S. Trametinib and Hydroxychloroquine (HCQ) Combination Treatment in KRAS-Mutated Advanced Pancreatic Adenocarcinoma: Detailed Description of Two Cases. J Gastrointest Cancer 52, 374-380, doi:10.1007/s12029-020-00556-z (2021).
46 Silvis, M. R. et al. MYC-mediated resistance to trametinib and HCQ in PDAC is overcome by CDK4/6 and lysosomal inhibition. J Exp Med 220, doi:10.1084/jem.20221524 (2023).
47 Yamamoto, K. et al. Targeting autophagy as a therapeutic strategy against pancreatic cancer. J Gastroenterol 57, 603-618, doi:10.1007/s00535-022-01889-1 (2022).
48 Qiao, Y. et al. Autophagy Inhibition by Targeting PIKfyve Potentiates Response to Immune Checkpoint Blockade in Prostate Cancer. Nat Cancer 2, 978-993, doi:10.1038/s43018-021-00237-1 (2021).
49 Krishna, S. et al. PIKfyve Regulates Vacuole Maturation and Nutrient Recovery following Engulfment. Dev Cell 38, 536-547, doi:10.1016/j.devcel.2016.08.001 (2016).
50 Bao, Y. et al. Targeting the lipid kinase PIKfyve upregulates surface expression of MHC class I to augment cancer immunotherapy. Proc Natl Acad Sci USA 120, e2314416120, doi:10.1073/pnas.2314416120 (2023).
51 Gayle, S. et al. Identification of apilimod as a first-in-class PIKfyve kinase inhibitor for treatment of B-cell non-Hodgkin lymphoma. Blood 129, 1768-1778, doi:10.1182/blood-2016-09-736892 (2017).
52 de Campos, C. B. et al. Identification of PIKfyve kinase as a target in multiple myeloma. Haematologica 105, 1641-1649, doi:10.3324/haematol.2019.222729 (2020).
53 Pili, R., Carducci, M., Brown, P. & Hurwitz, H. An open-label study to determine the maximum tolerated dose of the multitargeted tyrosine kinase inhibitor CEP-11981 in patients with advanced cancer. Invest New Drugs 32, 1258-1268, doi:10.1007/s10637-014-0147-9 (2014).
54 Harb, W. A. et al. Phase 1 Clinical Safety, Pharmacokinetics (PK), and Activity of Apilimod Dimesylate (LAM-002A), a First-in-Class Inhibitor of Phosphatidylinositol-3-Phosphate 5-Kinase (PIKfyve), in Patients with Relapsed or Refractory B-Cell Malignancies. Blood 130, 4119-4119, doi:10.1182/blood.V130.Suppl_1.4119.4119 (2017).
55 Huang, A., Garraway, L. A., Ashworth, A. & Weber, B. Synthetic lethality as an engine for cancer drug target discovery. Nat Rev Drug Discov 19, 23-38, doi:10.1038/s41573-019-0046-z (2020).
56 Weber, R. A. et al. Maintaining Iron Homeostasis Is the Key Role of Lysosomal Acidity for Cell Proliferation. Mol Cell 77, 645-655 e647, doi:10.1016/j.molcel.2020.01.003 (2020).
57 Sunami, Y., Rebelo, A. & Kleeff, J. Lipid Metabolism and Lipid Droplets in Pancreatic Cancer and Stellate Cells. Cancers (Basel) 10, doi:10.3390/cancers10010003 (2017).
58 Walter, K. et al. Serum fatty acid synthase as a marker of pancreatic neoplasia. Cancer Epidemiol Biomarkers Prev 18, 2380-2385, doi:10.1158/1055-9965.EPI-09-0144 (2009).
59 Hatzivassiliou, G. et al. ATP citrate lyase inhibition can suppress tumor cell growth. Cancer Cell 8, 311-321, doi:10.1016/j.ccr.2005.09.008 (2005).
60 Ikonomov, O. C., Sbrissa, D. & Shisheva, A. Small molecule PIKfyve inhibitors as cancer therapeutics: Translational promises and limitations. Toxicol Appl Pharmacol 383, 114771, doi:10.1016/j.taap.2019.114771 (2019).
61 Kaizuka, T. et al. An Autophagic Flux Probe that Releases an Internal Control. Mol Cell 64, 835-849, doi:10.1016/j.molcel.2016.09.037 (2016).
62 Li, C. et al. Discovery of a First-in-Class Degrader for the Lipid Kinase PIKfyve. J Med Chem 66, 12432-12445, doi:10.1021/acs.jmedchem.3c00912 (2023).
63 Reed, B. D., Charos, A. E., Szekely, A. M., Weissman, S. M. & Snyder, M. Genome-wide occupancy of SREBP1 and its partners NFY and SP1 reveals novel functional roles and combinatorial regulation of distinct classes of genes. PLoS Genet 4, e1000133, doi:10.1371/journal.pgen.1000133 (2008).
64 Collins, M. A. et al. Oncogenic Kras is required for both the initiation and maintenance of pancreatic cancer in mice. J Clin Invest 122, 639-653, doi:10.1172/JC159227 (2012).
65 Schrezenmeier, E. & Dorner, T. Mechanisms of action of hydroxychloroquine and chloroquine: implications for rheumatology. Nat Rev Rheumatol 16, 155-166, doi:10.1038/s41584-020-0372-x (2020).
66 Ravichandran, M. et al. Coordinated Transcriptional and Catabolic Programs Support Iron-Dependent Adaptation to RAS-MAPK Pathway Inhibition in Pancreatic Cancer. Cancer Discov 12, 2198-2219, doi:10.1158/2159-8290.CD-22-0044 (2022).
67 Hosios, A. M. et al. mTORC1 regulates a lysosome-dependent adaptive shift in intracellular lipid species. Nat Metab 4, 1792-1811, doi:10.1038/s42255-022-00706-6 (2022).
68 Wasko, U. N. et al. Tumor-selective effects of active RAS inhibition in pancreatic ductal adenocarcinoma. bioRxiv, 2023.2012.2003.569791, doi:10.1101/2023.12.03.569791 (2023).
69 Kim, D. et al. Pan-KRAS inhibitor disables oncogenic signalling and tumour growth. Nature 619, 160-166, doi:10.1038/s41586-023-06123-3 (2023).
70 Li, C. et al. Identification of pancreatic cancer stem cells. Cancer Res 67, 1030-1037, doi:10.1158/0008-5472.CAN-06-2030 (2007).
71 Jafari, R. et al. The cellular thermal shift assay for evaluating drug target interactions in cells. Nat Protoc 9, 2100-2122, doi:10.1038/nprot.2014.138 (2014).
72 Zheng, S. et al. SynergyFinder Plus: Toward Better Interpretation and Annotation of Drug Combination Screening Datasets. Genomics Proteomics Bioinformatics 20, 587-596, doi:10.1016/j.gpb.2022.01.004 (2022).
73 Birsoy, K. et al. An Essential Role of the Mitochondrial Electron Transport Chain in Cell Proliferation Is to Enable Aspartate Synthesis. Cell 162, 540-551, doi:10.1016/j.cell.2015.07.016 (2015).
74 Kechin, A., Boyarskikh, U., Kel, A. & Filipenko, M. cutPrimers: A New Tool for Accurate Cutting of Primers from Reads of Targeted Next Generation Sequencing. J Comput Biol 24, 1138-1143, doi:10.1089/cmb.2017.0096 (2017).
75 Langmead, B. & Salzberg, S. L. Fast gapped-read alignment with Bowtie 2. Nat Methods 9, 357-359, doi:10.1038/nmeth.1923 (2012).
76 Li, W. et al. MAGeCK enables robust identification of essential genes from genome-scale CRISPR/Cas9 knockout screens. Genome Biol 15, 554, doi:10.1186/s13059-014-0554-4 (2014).
77 Rossiter, N. J. et al. CRISPR screens in physiologic medium reveal conditionally essential genes in human cells. Cell Metab 33, 1248-1263 e1249, doi:10.1016/j.cmet.2021.02.005 (2021).
78 Bray, N. L., Pimentel, H., Melsted, P. & Pachter, L. Near-optimal probabilistic RNA-seq quantification. Nat Biotechnol 34, 525-527, doi:10.1038/nbt.3519 (2016).
79 Law, C. W., Chen, Y., Shi, W. & Smyth, G. K. voom: Precision weights unlock linear model analysis tools for RNA-seq read counts. Genome Biol 15, R29, doi:10.1186/gb-2014-15-2-r29 (2014).
80 Ritchie, M. E. et al. limma powers differential expression analyses for RNA-sequencing and microarray studies. Nucleic Acids Res 43, e47, doi:10.1093/nar/gkv007 (2015).
81 Robinson, M. D. & Oshlack, A. A scaling normalization method for differential expression analysis of RNA-seq data. Genome Biol 11, R25, doi:10.1186/gb-2010-11-3-r25 (2010).
82 Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).
83 Liberzon, A. et al. The Molecular Signatures Database (MSigDB) hallmark gene set collection. Cell Syst 1, 417-425, doi:10.1016/j.cels.2015.12.004 (2015).
84 Sergushichev, A. A. An algorithm for fast preranked gene set enrichment analysis using cumulative statistic calculation. bioRxiv, 060012, doi:10.1101/060012 (2016).
85 Replogle, J. M. et al. Mapping information-rich genotype-phenotype landscapes with genome-scale Perturb-seq. Cell 185, 2559-2575 e2528, doi:10.1016/j.cell.2022.05.013 (2022).
86 Thakore, P. I. et al. Highly specific epigenome editing by CRISPR-Cas9 repressors for silencing of distal regulatory elements. Nat Methods 12, 1143-1149, doi:10.1038/nmeth.3630 (2015).
87 Kerk, S. A. et al. Metabolic requirement for GOT2 in pancreatic cancer depends on environmental context. Elife 11, doi:10.7554/eLife.73245 (2022).
88 Bates, D., Mächler, M., Bolker, B. & Walker, S. Fitting Linear Mixed-Effects Models Using lme4. Journal of Statistical Software 67, 1-48, doi:10.18637/jss.v067.i01 (2015).

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

What is claimed is:

1. A method of treating cancer in a subject in need thereof, the method comprising administering to the subject a therapeutically effective amount of 13-isobutyl-4-methyl-10-(pyrimidin-2-ylamino)-1,2,4,7,8,13-hexahydro-6H-indazolo[5,4-a]pyrrolo[3,4-c]carbazol-6-one (ESK981), or pharmaceutically acceptable salt or solvate thereof, wherein the cancer is neuroendocrine prostate cancer (NEPC), pancreatic ductal adenocarcinoma (PDAC), pancreatic neuroendocrine tumors (PNETs), pancreatic neuroendocrine carcinomas (NECs), colorectal cancer (CRC), or non-small cell lung cancer (NSCLC).

2. The method of claim 1, wherein the cancer is NEPC, PDAC, PNETs, or NECs.

3. The method of claim 1, wherein ESK981, or pharmaceutically acceptable salt or solvate thereof, is:
(i) a crystalline para-toluenesulfonic acid (pTSA) salt having an ESK981:pTSA: ratio of 1:1, characterized as having an x-ray powder diffraction (XRPD) pattern comprising peaks at 5.37, 6.79, 13.64, 22.58, and 25.54 degrees 2Θ using Cu Kα radiation, wherein the 2Θ values are ±0.2 degrees 2Θ (ESK981 pTSA-A₁);
(ii) a crystalline pTSA salt having an ESK981:pTSA ratio of 1:2, characterized as having an XRPD pattern comprising peaks at 5.63, 8.48, 12.46, 18.21, and 23.95 degrees 2Θ using Cu Kα radiation, wherein the 2Θ values are ±0.2 degrees 2Θ (ESK981 pTSA-A₂);
(iii) a crystalline pTSA salt having an ESK981:pTSA ratio of 1:2, characterized as having an XRPD pattern comprising peaks at 3.75, 5.63, 8.40, 11.31, and 15.12 degrees 2Θ using Cu Kα radiation, wherein the 2Θ values are ±0.2 degrees 2Θ (ESK981 pTSA-B₂);
(iv) a crystalline pTSA salt having an ESK981:pTSA ratio of 1:2, characterized as having an XRPD pattern comprising peaks at 5.72, 8.49, 11.37, 13.26, and 16.95 degrees 2Θ using Cu Kα radiation, wherein the 2Θ values are ±0.2 degrees 2Θ (ESK981 pTSA-C₂); or
(v) a crystalline acetic acid (CH₃CO₂H) salt having an ESK981:CH₃CO₂H ratio of 1:2, characterized as having an XRPD pattern comprising peaks at 6.17, 6.44, 14.36, 20.69, and 26.09 degrees 2Θ using Cu Kα radiation, wherein the 2Θ values are ±0.2 degrees 2Θ (ESK981 AA).

4. The method of claim 1, further comprising administering a therapeutically effective amount of an anticancer agent to the subject in combination with ESK981, or pharmaceutically acceptable salt or solvate thereof.

5. The method of claim 4, wherein the anticancer agent comprises a RAS inhibitor.

6. The method of claim 4, wherein the RAS inhibitor is a pan-RAS inhibitor.

7. The method of claim 4, wherein the RAS inhibitor is a KRAS inhibitor.

8. The method of claim 4, wherein the KRAS inhibitor is a KRAS^{G12C} inhibitor.

9. The method of claim 4, wherein the KRAS inhibitor is a KRAS^{G12D} inhibitor.

10. The method of claim 4, wherein the RAS inhibitor is MRTX849 (Adagrasib), AMG 510 (Sotorasib), BBO-8520, BBO-8956, D3S-001 (Elisrasib), GDC-6036 (Divarasib), D-1553 (Garsorasib), GH35, JAB-21822 (Glecirasib), IBI351/GFH925 (Fulzerasib), LY3499446, MK-1084, LY3537982 (Olomorasib), JDQ443 (Opnurasib), RMC-4998, RMC-6291 (Elironrasib), ASP3082, HRS-4642, MRTX1133, RMC-9805 (Zoldonrasib), RMC-5127, ACBI3, BI-2493, BI-2865, RMC-6236 (Daraxonrasib), RMC-7977, BEBT-607, BI 1823911, BPI-421286, FMC-376, GEC255, HBI-2438, HS-10370, JNJ-74699157, YL-15293, GFH357/VS-7375, INCB161734, JAB-22000, LY3962673, QTX3046, TSN1611, RMC-8839, RMC-0708, BI 3706674, JAB-23E73, LY4066434, PF-07934040, QTX3034, GFH547, YL-17231, BPI-585725, or ERAS-4001.

11. The method of claim 2, wherein the RAS inhibitor is AMG510 (Sotorasib), MRTX849 (Adagrasib), or MRTX-1133.

12. A method of treating a subject having NEPC, PDAC, PNETs, NECs, CRC, or NSCLC, the method comprising:
(a) determining whether a KRAS mutation and/or a Trp53 mutation is present or absent in a biological sample taken from the subject; and
(b) administering a therapeutically effective amount of ESK981, or pharmaceutically acceptable salt or solvate thereof, to the subject if a KRAS mutation and/or a Trp53 mutation is present in the biological sample.

13. The method of claim 12, wherein ESK981, or pharmaceutically acceptable salt or solvate thereof, is ESK981 pTSA-A₁, ESK981 pTSA-A₂, ESK981 pTSA-B₂, ESK981 pTSA-C₂, or ESK981 AA.

14. The method of claim 12, further comprising administering a therapeutically effective amount of an anticancer agent to the subject in combination with ESK981, or pharmaceutically acceptable salt or solvate thereof.

15. The method of claim 14, wherein the anticancer agent comprises a RAS inhibitor.

16. The method of claim 15, wherein the KRAS mutation is Kras^{G12D}.

17. The method of claim 16, wherein the RAS inhibitor is a a KRAS^{G12D} inhibitor

18. The method of claim 15, wherein the RAS inhibitor is the RAS inhibitor is MRTX849 (Adagrasib), AMG 510 (Sotorasib), BBO-8520, BBO-8956, D3S-001 (Elisrasib), GDC-6036 (Divarasib), D-1553 (Garsorasib), GH35, JAB-21822 (Glecirasib), IBI351/GFH925 (Fulzerasib), LY3499446, MK-1084, LY3537982 (Olomorasib), JDQ443 (Opnurasib), RMC-4998, RMC-6291 (Elironrasib), ASP3082, HRS-4642, MRTX1133, RMC-9805 (Zoldonrasib), RMC-5127, ACBI3, BI-2493, BI-2865, RMC-6236 (Daraxonrasib), RMC-7977, BEBT-607, BI 1823911, BPI-421286, FMC-376, GEC255, HBI-2438, HS-10370, JNJ-74699157, YL-15293, GFH357/VS-7375, INCB161734, JAB-22000, LY3962673, QTX3046, TSN1611, RMC-8839, RMC-0708, BI 3706674, JAB-23E73, LY4066434, PF-07934040, QTX3034, GFH547, YL-17231, BPI-585725, or ERAS-4001

19. The method of claim 18, wherein the RAS inhibitor is AMG510 (Sotorasib), MRTX849 (Adagrasib), or MRTX-1133.

20. A kit for carrying out the methods claim 1, the kit comprising ESK981, or a pharmaceutically acceptable salt and/or solvate thereof, and a label with instructions for how to administer ESK981, or a pharmaceutically acceptable salt and/or solvate thereof, to a subject.
